SEQUENCE LISTING (299 sequences)

SEQ ID NO: 1 - RNA, 22 nt, Homo sapiens
ugagguaguagauuguauaguu

SEQ ID NO: 2 - RNA, 22 nt, Homo sapiens
aacccguagauccgaacuugug

SEQ ID NO: 3 - RNA, 23 nt, Homo sapiens
agcagcauuguacagggcuauga

SEQ ID NO: 4 - RNA, 23 nt, Homo sapiens
agcagcauuguacagggcuauca

SEQ ID NO: 5 - RNA, 23 nt, Homo sapiens
uacccuguagaaccgaauuugug

SEQ ID NO: 6 - RNA, 22 nt, Homo sapiens
uuuccggcucgcgugggugugu

SEQ ID NO: 7 - RNA, 24 nt, Homo sapiens
guggguaggguuugggggagagcg

SEQ ID NO: 8 - RNA, 20 nt, Homo sapiens
uaaggcacgcggugaaugcc

SEQ ID NO: 9 - RNA, 22 nt, Homo sapiens
ucacaagucaggcucuugggac

SEQ ID NO: 10 - RNA, 22 nt, Homo sapiens
cuuggcaccuagcaagcacuca

SEQ ID NO: 11 - RNA, 19 nt, Homo sapiens
ggagauggagguugcagug

SEQ ID NO: 12 - RNA, 22 nt, Homo sapiens
ucugggcaacaaagugagaccu

SEQ ID NO: 13 - RNA, 22 nt, Homo sapiens
aagcccuuaccccaaaaaguau

SEQ ID NO: 14 - RNA, 22 nt, Homo sapiens
aagcccuuaccccaaaaagcau

SEQ ID NO: 15 - RNA, 19 nt, Homo sapiens
uggauuuuuggaucaggga

SEQ ID NO: 16 - RNA, 22 nt, Homo sapiens
cagugcaauguuaaaagggcau

SEQ ID NO: 17 - RNA, 22 nt, Homo sapiens
cagugcaaugaugaaagggcau

SEQ ID NO: 18 - RNA, 22 nt, Homo sapiens
uaacagucuacagccauggucg

SEQ ID NO: 19 - RNA, 22 nt, Homo sapiens
accguggcuuucgauuguuacu

SEQ ID NO: 20 - RNA, 23 nt, Homo sapiens
agcugguguugugaaucaggccg

SEQ ID NO: 21 - RNA, 23 nt, Homo sapiens
uguaguguuuccuacuuuaugga

SEQ ID NO: 22 - RNA, 22 nt, Homo sapiens
ucagugcacuacagaacuuugu

SEQ ID NO: 23 - RNA, 21 nt, Homo sapiens
ucagugcaugacagaacuugg

SEQ ID NO: 24 - RNA, 23 nt, Homo sapiens
uuaaugcuaaucgugauaggggu

SEQ ID NO: 25 - RNA, 22 nt, Homo sapiens
uagcagcacaucaugguuuaca

SEQ ID NO: 26 - RNA, 22 nt, Homo sapiens
accacugaccguugacuguacc

SEQ ID NO: 27 - RNA, 22 nt, Homo sapiens
aaccaucgaccguugaguggac

SEQ ID NO: 28 - RNA, 22 nt, Homo sapiens
aggggcuggcuuuccucugguc

SEQ ID NO: 29 - RNA, 22 nt, Homo sapiens
uggagagaaaggcaguuccuga

SEQ ID NO: 30 - RNA, 23 nt, Homo sapiens
uaaggugcaucuagugcagauag

SEQ ID NO: 31 - RNA, 23 nt, Homo sapiens
uaaggugcaucuagugcaguuag

SEQ ID NO: 32 - RNA, 21 nt, Homo sapiens
uagcagcacagaaauauuggc

SEQ ID NO: 33 - RNA, 22 nt, Homo sapiens
acaguagucugcacauugguua

SEQ ID NO: 34 - RNA, 23 nt, Homo sapiens
cccaguguucagacuaccuguuc

SEQ ID NO: 35 - RNA, 23 nt, Homo sapiens
ugugcaaauccaugcaaaacuga

SEQ ID NO: 36 - RNA, 22 nt, Homo sapiens
uucccuuugucauccuaugccu

SEQ ID NO: 37 - RNA, 22 nt, Homo sapiens
cugugcgugugacagcggcuga

SEQ ID NO: 38 - RNA, 21 nt, Homo sapiens
uaacagucuccagucacggcc

SEQ ID NO: 39 - RNA, 22 nt, Homo sapiens
acagcaggcacagacaggcagu

SEQ ID NO: 40 - RNA, 23 nt, Homo sapiens
agcuacauugucugcuggguuuc

SEQ ID NO: 41 - RNA, 21 nt, Homo sapiens
agcuacaucuggcuacugggu

SEQ ID NO: 42 - RNA, 21 nt, Homo sapiens
caagucacuagugguuccguu

SEQ ID NO: 43 - RNA, 21 nt, Homo sapiens
uucaaguaauucaggauaggu

SEQ ID NO: 44 - RNA, 22 nt, Homo sapiens
uagcaccaucugaaaucgguua

SEQ ID NO: 45 - RNA, 23 nt, Homo sapiens
cagugcaauaguauugucaaagc

SEQ ID NO: 46 - RNA, 22 nt, Homo sapiens
cuuucagucggauguuugcagc

SEQ ID NO: 47 - RNA, 22 nt, Homo sapiens
uguaaacauccucgacuggaag

SEQ ID NO: 48 - RNA, 22 nt, Homo sapiens
uguaaacauccuacacucagcu

SEQ ID NO: 49 - RNA, 22 nt, Homo sapiens
cugggagaaggcuguuuacucu

SEQ ID NO: 50 - RNA, 23 nt, Homo sapiens
uguaaacauccuacacucucagc

SEQ ID NO: 51 - RNA, 22 nt, Homo sapiens
uguaaacauccccgacuggaag

SEQ ID NO: 52 - RNA, 22 nt, Homo sapiens
cuuucagucggauguuuacagc

SEQ ID NO: 53 - RNA, 22 nt, Homo sapiens
uguaaacauccuugacuggaag

SEQ ID NO: 54 - RNA, 23 nt, Homo sapiens
agaagaaggcggucggucugcgg

SEQ ID NO: 55 - RNA, 22 nt, Homo sapiens
caccuugcgcuacucaggucug

SEQ ID NO: 56 - RNA, 21 nt, Homo sapiens
cacauuacacggucgaccucu

SEQ ID NO: 57 - RNA, 20 nt, Homo sapiens
acugccccaggugcugcugg

SEQ ID NO: 58 - RNA, 22 nt, Homo sapiens
aacacaccugguuaaccucuuu

SEQ ID NO: 59 - RNA, 23 nt, Homo sapiens
gcaaagcacacggccugcagaga

SEQ ID NO: 60 - RNA, 23 nt, Homo sapiens
ucccuguccuccaggagcucacg

SEQ ID NO: 61 - RNA, 22 nt, Homo sapiens
gcugacuccuaguccagggcuc

SEQ ID NO: 62 - RNA, 22 nt, Homo sapiens
caaucagcaaguauacugcccu

SEQ ID NO: 63 - RNA, 22 nt, Homo sapiens
uggcagugucuuagcugguugu

SEQ ID NO: 64 - RNA, 23 nt, Homo sapiens
uaggcagugucauuagcugauug

SEQ ID NO: 65 - RNA, 23 nt, Homo sapiens
aggcaguguaguuagcugauugc

SEQ ID NO: 66 - RNA, 20 nt, Homo sapiens
acuguaaacgcuuucugaug

SEQ ID NO: 67 - RNA, 22 nt, Homo sapiens
uuaucagaaucuccagggguac

SEQ ID NO: 68 - RNA, 18 nt, Homo sapiens
cuaagaaguugacugaag

SEQ ID NO: 69 - RNA, 22 nt, Homo sapiens
uuauaauacaaccugauaagug

SEQ ID NO: 70 - RNA, 22 nt, Homo sapiens
auauaauacaaccugcuaagug

SEQ ID NO: 71 - RNA, 22 nt, Homo sapiens
auaauacaaccugcuaagugcu

SEQ ID NO: 72 - RNA, 22 nt, Homo sapiens
acuggacuuggagucagaaggc

SEQ ID NO: 73 - RNA, 25 nt, Homo sapiens
acuggacuuggagucagaagagugg

SEQ ID NO: 74 - RNA, 20 nt, Homo sapiens
acuggacuuggagucagaaa

SEQ ID NO: 75 - RNA, 20 nt, Homo sapiens
acugggcuuggagucagaag

SEQ ID NO: 76 - RNA, 21 nt, Homo sapiens
acuggacuaggagucagaagg

SEQ ID NO: 77 - RNA, 22 nt, Homo sapiens
aggugcuccaggcuggcucaca

SEQ ID NO: 78 - RNA, 23 nt, Homo sapiens
aucaacagacauuaauugggcgc

SEQ ID NO: 79 - RNA, 22 nt, Homo sapiens
acuggacuuagggucagaaggc

SEQ ID NO: 80 - RNA, 23 nt, Homo sapiens
ugaggggcagagagcgagacuuu

SEQ ID NO: 81 - RNA, 22 nt, Homo sapiens
cagcagcaauucauguuuugaa

SEQ ID NO: 82 - RNA, 23 nt, Homo sapiens
aaugacacgaucacucccguuga

SEQ ID NO: 83 - RNA, 17 nt, Homo sapiens
uggagagaaaggcagua

SEQ ID NO: 84 - RNA, 20 nt, Homo sapiens
cccugagacccuaaccuuaa

SEQ ID NO: 85 - RNA, 20 nt, Homo sapiens
ggauccgagucacggcacca

SEQ ID NO: 86 - RNA, 22 nt, Homo sapiens
aaaccguuaccauuacugaguu

SEQ ID NO: 87 - RNA, 22 nt, Homo sapiens
gcuaaggaaguccugugcucag

SEQ ID NO: 88 - RNA, 23 nt, Homo sapiens
uagugcaauauugcuuauagggu

SEQ ID NO: 89 - RNA, 21 nt, Homo sapiens
gcaguccaugggcauauacac

SEQ ID NO: 90 - RNA, 23 nt, Homo sapiens
ugggccagggagcagcugguggg

SEQ ID NO: 91 - RNA, 23 nt, Homo sapiens
ugggcugagggcaggaggccugu

SEQ ID NO: 92 - RNA, 22 nt, Homo sapiens
uggggugcccacuccgcaaguu

SEQ ID NO: 93 - RNA, 22 nt, Homo sapiens
gagcaggcgaggcugggcugaa

SEQ ID NO: 94 - RNA, 22 nt, Homo sapiens
caggaggcagugggcgagcagg

SEQ ID NO: 95 - RNA, 23 nt, Homo sapiens
uguagagcagggagcaggaagcu

SEQ ID NO: 96 - RNA, 19 nt, Homo sapiens
cauccguccgucuguccac

SEQ ID NO: 97 - RNA, 22 nt, Homo sapiens
aaucguacagggucauccacuu

SEQ ID NO: 98 - RNA, 21 nt, Homo sapiens
cagcagcacacugugguuugu

SEQ ID NO: 99 - RNA, 24 nt, Homo sapiens
agggcuggacucagcggcggagcu

SEQ ID NO: 100 - RNA, 22 nt, Homo sapiens
agggggauggcagagcaaaauu

SEQ ID NO: 101 - RNA, 23 nt, Homo sapiens
uagcagcgggaacaguucugcag

SEQ ID NO: 102 - RNA, 22 nt, Homo sapiens
agacccuggucugcacucuauc

SEQ ID NO: 103 - RNA, 22 nt, Homo sapiens
gggagccaggaaguauugaugu

SEQ ID NO: 104 - RNA, 22 nt, Homo sapiens
uucagaucccagcggugccucu

SEQ ID NO: 105 - RNA, 22 nt, Homo sapiens
caugccuugaguguaggaccgu

SEQ ID NO: 106 - RNA, 23 nt, Homo sapiens
ucggggaucaucaugucacgaga

SEQ ID NO: 107 - RNA, 20 nt, Homo sapiens
aacucuagccugagcaacag

SEQ ID NO: 108 - RNA, 22 nt, Homo sapiens
uuaugguuugccugggacugag

SEQ ID NO: 109 - RNA, 21 nt, Homo sapiens
agggggaaaguucuauagucc

SEQ ID NO: 110 - RNA, 21 nt, Homo sapiens
uggguuuacguugggagaacu

SEQ ID NO: 111 - RNA, 23 nt, Homo sapiens
caggcagaaguggggcugacagg

SEQ ID NO: 112 - RNA, 21 nt, Homo sapiens
aauggcgccacuaggguugug

SEQ ID NO: 113 - RNA, 22 nt, Homo sapiens
uucauuugccucccagccuaca

SEQ ID NO: 114 - RNA, 23 nt, Homo sapiens
aggaagcccuggaggggcuggag

SEQ ID NO: 115 - RNA, 22 nt, Homo sapiens
auaguccgaguaacgucggggc

SEQ ID NO: 116 - RNA, 22 nt, Homo sapiens
caacuagacugugagcuucuag

SEQ ID NO: 117 - RNA, 23 nt, Homo sapiens
aaggagcuuacaaucuagcuggg

SEQ ID NO: 118 - RNA, 22 nt, Homo sapiens
ugcggggcuagggcuaacagca

SEQ ID NO: 119 - RNA, 22 nt, Homo sapiens
ugagaccucuggguucugagcu

SEQ ID NO: 120 - RNA, 22 nt, Homo sapiens
auaaagcuagauaaccgaaagu

SEQ ID NO: 121 - RNA, 23 nt, Homo sapiens
ucuuugguuaucuagcuguauga

SEQ ID NO: 122 - RNA, 22 nt, Homo sapiens
caagcucgcuucuaugggucug

SEQ ID NO: 123 - RNA, 22 nt, Homo sapiens
aacccguagauccgaucuugug

SEQ ID NO: 124 - RNA, 22 nt, Homo sapiens
caagcucgugucuguggguccg

SEQ ID NO: 125 - RNA, 22 nt, Homo sapiens
auaugccauguuggugugcugc

SEQ ID NO: 126 - RNA, 22 nt, Homo sapiens
ucgaggcuagagucacgcuugg

SEQ ID NO: 127 - RNA, 22 nt, Homo sapiens
uggacuuggagucaggaggccu

SEQ ID NO: 128 - RNA, 22 nt, Homo sapiens
uggugugcuagaguacucgaag

SEQ ID NO: 129 - RNA, 22 nt, Homo sapiens
ugugccauguuggugugcugca

SEQ ID NO: 130 - RNA, 21 nt, Homo sapiens
cgcgggugcuuacugacccuu

SEQ ID NO: 131 - RNA, 23 nt, Homo sapiens
cgggucggaguuagcucaagcgg

SEQ ID NO: 132 - RNA, 27 nt, Homo sapiens
auugaucaucgacacuucgaacgcaau

SEQ ID NO: 133 - RNA, 21 nt, Homo sapiens
acaugaaaaggggagagggca

SEQ ID NO: 134 - RNA, 22 nt, Homo sapiens
agcugguuugauggggagccau

SEQ ID NO: 135 - RNA, 21 nt, Homo sapiens
aucccggacgagcccccauuu

SEQ ID NO: 136 - RNA, 22 nt, Homo sapiens
cacugauuaucgaggcgauucu

SEQ ID NO: 137 - RNA, 20 nt, Homo sapiens
gaaagcugagcgugaacgug

SEQ ID NO: 138 - RNA, 20 nt, Homo sapiens
gaacccuacuccugguacca

SEQ ID NO: 139 - RNA, 22 nt, Homo sapiens
gacgugagggggugcuacauac

SEQ ID NO: 140 - RNA, 22 nt, Homo sapiens
gcuuguugaaaggcgcgggcuc

SEQ ID NO: 141 - RNA, 23 nt, Homo sapiens
ggcugguccgaagguagugaguu

SEQ ID NO: 142 - RNA, 25 nt, Homo sapiens
ggcugguccgagugcagugguguuu

SEQ ID NO: 143 - RNA, 23 nt, Homo sapiens
guuccuguuggccgaguggagac

SEQ ID NO: 144 - RNA, 18 nt, Homo sapiens
uguggguucgaguuccau

SEQ ID NO: 145 - RNA, 25 nt, Homo sapiens
cccccacaaccgcgcuugacuagcu

SEQ ID NO: 146 - RNA, 22 nt, Homo sapiens
uggacucugagaaaggaguaug

SEQ ID NO: 147 - RNA, 21 nt, Homo sapiens
uacuccuuaucagacuccaua

SEQ ID NO: 148 - RNA, 22 nt, Homo sapiens
uggcagugucuuagcugguugu

SEQ ID NO: 149 - RNA, 22 nt, Homo sapiens
caaucagcaaguauacugcccu

SEQ ID NO: 150 - RNA, 22 nt, Homo sapiens
uggacucugagaaaggaguaug

SEQ ID NO: 151 - RNA, 21 nt, Homo sapiens
uacuccuuaucagacuccaua

SEQ ID NO: 152 - RNA, 87 nt, Homo sapiens
ucagagugagguaguagauuguauaguugugggguagugauuuuacccuguucaggagauaacuauacaaucuauugccuucccuga

SEQ ID NO: 153 - RNA, 83 nt, Homo sapiens
ugugggaugagguaguagauuguauaguuuuagggucauaccccaucuuggagauaacuauacagucuacugucuuucccacg

SEQ ID NO: 154 - RNA, 80 nt, Homo sapiens
ccuguugccacaaacccguagauccgaacuugugguauuaguccgcacaagcuuguaucuauagguaugugucuguuagg

SEQ ID NO: 155 - RNA, 78 nt, Homo sapiens
uugugcuuucagcuucuuuacagugcugccuuguagcauucaggucaagcagcauuguacagggcuaugaaagaacca

SEQ ID NO: 156 - RNA, 78 nt, Homo sapiens
uacugcccucggcuucuuuacagugcugccuuguugcauauggaucaagcagcauuguacagggcuaugaaggcauug

SEQ ID NO: 157 - RNA, 81 nt, Homo sapiens
cucucugcuuucagcuucuuuacaguguugccuuguggcauggaguucaagcagcauuguacagggcuaucaaagcacaga

SEQ ID NO: 158 - RNA, 110 nt, Homo sapiens
ccagagguuguaacguugucuauauauacccuguagaaccgaauuugugugguauccguauagucacagauucgauucuaggggaauauauggucgaugcaaaaacuuca

SEQ ID NO: 159 - RNA, 69 nt, Homo sapiens
gcugcuggacccacccggccgggaauagugcuccugguuguuuccggcucgcgugggugugucggcggc

SEQ ID NO: 160 - RNA, 69 nt, Homo sapiens
guggguaggguuugggggagagcgugggcugggguucagggacacccucucaccacugcccucccacag

SEQ ID NO: 161 - RNA, 85 nt, Homo sapiens
aggccucucucuccguguucacagcggaccuugauuuaaauguccauacaauuaaggcacgcggugaaugccaagaauggggcug

SEQ ID NO: 162 - RNA, 87 nt, Homo sapiens
ugagggccccucugcguguucacagcggaccuugauuuaaugucuauacaauuaaggcacgcggugaaugccaagagaggcgccucc

SEQ ID NO: 163 - RNA, 109 nt, Homo sapiens
aucaagauuagaggcucugcucuccguguucacagcggaccuugauuuaaugucauacaauuaaggcacgcggugaaugccaagagcggagccuacggcugcacuugaa

SEQ ID NO: 164 - RNA, 89 nt, Homo sapiens
accagacuuuuccuagucccugagacccuaacuugugagguauuuuaguaacaucacaagucaggcucuugggaccuaggcggagggga

SEQ ID NO: 165 - RNA, 86 nt, Homo sapiens
cacccagaucagugcuuggcaccuagcaagcacucaguaaauauuuguugagugccugcuaugugccaggcauugugcugagggcu

SEQ ID NO: 166 - RNA, 99 nt, Homo sapiens
aggugggaggauugcuugagccugggagauggagguugcagugagcugagaucacgcaacugcacccccagccugggccauagagucaguccuugucuc

SEQ ID NO: 167 - RNA, 88 nt, Homo sapiens
uuugggaggccgaggcuggugcaucacuugagcccagcaauuugagaccaaucugggcaacaaagugagaccuccgucucuacaaaga

SEQ ID NO: 168 - RNA, 84 nt, Homo sapiens
uguagagauaggaucucacuuuguugcccaggcuggucucaaacuccuggucugggcaacaaagugagaccuuaucucuacaag

SEQ ID NO: 169 - RNA, 72 nt, Homo sapiens
ggaucuuuuugcggucugggcuugcuguuccucucaacaguagucaggaagcccuuaccccaaaaaguaucu

SEQ ID NO: 170 - RNA, 90 nt, Homo sapiens
ugcccuucgcgaaucuuuuugcggucugggcuugcuguacauaacucaauagccggaagcccuuaccccaaaaagcauuugcggagggcg

SEQ ID NO: 171 - RNA, 78 nt, Homo sapiens
gagcgucacguugacacucaaaaaguuucagauuuuggaacauuucggauuuuggauuuuuggaucagggaugcucaa

SEQ ID NO: 172 - RNA, 89 nt, Homo sapiens
ugcugcuggccagagcucuuuucacauugugcuacugucugcaccugucacuagcagugcaauguuaaaagggcauuggccguguagug

SEQ ID NO: 173 - RNA, 82 nt, Homo sapiens
ggccugcccgacacucuuucccuguugcacuacuauaggccgcugggaagcagugcaaugaugaaagggcaucggucagguc

SEQ ID NO: 174 - RNA, 101 nt, Homo sapiens
ccgcccccgcgucuccagggcaaccguggcuuucgauuguuacugugggaacuggagguaacagucuacagccauggucgccccgcagcacgcccacgcgc

SEQ ID NO: 175 - RNA, 84 nt, Homo sapiens
cguugcugcagcugguguugugaaucaggccgacgagcagcgcauccucuuacccggcuauuucacgacaccaggguugcauca

SEQ ID NO: 176 - RNA, 99 nt, Homo sapiens
cccuggcauggugugguggggcagcugguguugugaaucaggccguugccaaucagagaacggcuacuucacaacaccagggccacaccacacuacagg

SEQ ID NO: 177 - RNA, 87 nt, Homo sapiens
gacagugcagucacccauaaaguagaaagcacuacuaacagcacuggaggguguaguguuuccuacuuuauggaugaguguacugug

SEQ ID NO: 178 - RNA, 68 nt, Homo sapiens
gaggcaaaguucugagacacuccgacucugaguaugauagaagucagugcacuacagaacuuugucuc

SEQ ID NO: 179 - RNA, 87 nt, Homo sapiens
ugucccccccggcccagguucugugauacacuccgacucgggcucuggagcagucagugcaugacagaacuugggcccggaaggacc

SEQ ID NO: 180 - RNA, 65 nt, Homo sapiens
cuguuaaugcuaaucgugauagggguuuuugccuccaacugacuccuacauauuagcauuaacag

SEQ ID NO: 181 - RNA, 98 nt, Homo sapiens
uugaggccuuaaaguacuguagcagcacaucaugguuuacaugcuacagucaagaugcgaaucauuauuugcugcucuagaaauuuaaggaaauucau

SEQ ID NO: 182 - RNA, 110 nt, Homo sapiens
agaagggcuaucaggccagccuucagaggacuccaaggaacauucaacgcugucggugaguuugggauuugaaaaaaccacugaccguugacuguaccuugggguccuua

SEQ ID NO: 183 - RNA, 110 nt, Homo sapiens
cggaaaauuugccaaggguuugggggaacauucaaccugucggugaguuugggcagcucaggcaaaccaucgaccguugaguggacccugaggccuggaauugccauccu

SEQ ID NO: 184 - RNA, 82 nt, Homo sapiens
agggggcgagggauuggagagaaaggcaguuccugaugguccccuccccaggggcuggcuuuccucugguccuucccuccca

SEQ ID NO: 185 - RNA, 71 nt, Homo sapiens
uguucuaaggugcaucuagugcagauagugaaguagauuagcaucuacugcccuaagugcuccuucuggca

SEQ ID NO: 186 - RNA, 71 nt, Homo sapiens
uguguuaaggugcaucuagugcaguuagugaagcagcuuagaaucuacugcccuaaaugccccuucuggca

SEQ ID NO: 187 - RNA, 87 nt, Homo sapiens
agcuucccuggcucuagcagcacagaaauauuggcacagggaagcgagucugccaauauuggcugugcugcuccaggcaggguggug

SEQ ID NO: 188 - RNA, 110 nt, Homo sapiens
aggaagcuucuggagauccugcuccgucgccccaguguucagacuaccuguucaggacaaugccguuguacaguagucugcacauugguuagacugggcaagggagagca

SEQ ID NO: 189 - RNA, 71 nt, Homo sapiens
gccaacccaguguucagacuaccuguucaggaggcucucaauguguacaguagucugcacauugguuaggc

SEQ ID NO: 190 - RNA, 87 nt, Homo sapiens
cacuguucuaugguuaguuuugcagguuugcauccagcugugugauauucugcugugcaaauccaugcaaaacugacugugguagug

SEQ ID NO: 191 - RNA, 96 nt, Homo sapiens
acauugcuacuuacaauuaguuuugcagguuugcauuucagcguauauauguauauguggcugugcaaauccaugcaaaacugauugugauaaugu

SEQ ID NO: 192 - RNA, 110 nt, Homo sapiens
ggcuacagucuuucuucaugugacucguggacuucccuuugucauccuaugccugagaauauaugaaggaggcugggaaggcaaagggacguucaauugucaucacuggc

SEQ ID NO: 193 - RNA, 110 nt, Homo sapiens
acccggcagugccuccaggcgcagggcagccccugcccaccgcacacugcgcugccccagacccacugugcgugugacagcggcugaucugugccugggcagcgcgaccc

SEQ ID NO: 194 - RNA, 110 nt, Homo sapiens
cggggcaccccgcccggacagcgcgccggcaccuuggcucuagacugcuuacugcccgggccgcccucaguaacagucuccagucacggccaccgacgccuggccccgcc

SEQ ID NO: 195 - RNA, 110 nt, Homo sapiens
ggccuggcuggacagaguugucaugugucugccugucuacacuugcugugcagaacauccgcucaccuguacagcaggcacagacaggcagucacaugacaacccagccu

SEQ ID NO: 196 - RNA, 110 nt, Homo sapiens
ugaacauccaggucuggggcaugaaccuggcauacaauguagauuucuguguucguuaggcaacagcuacauugucugcuggguuucaggcuaccuggaaacauguucuc

SEQ ID NO: 197 - RNA, 110 nt, Homo sapiens
gcugcuggaagguguagguacccucaauggcucaguagccaguguagauccugucuuucguaaucagcagcuacaucuggcuacugggucucugauggcaucuucuagcu

SEQ ID NO: 198 - RNA, 81 nt, Homo sapiens
gggcuuucaagucacuagugguuccguuuaguagaugauugugcauuguuucaaaauggugcccuagugacuacaaagccc

SEQ ID NO: 199 - RNA, 77 nt, Homo sapiens
ccgggacccaguucaaguaauucaggauagguugugugcuguccagccuguucuccauuacuuggcucggggaccgg

SEQ ID NO: 200 - RNA, 64 nt, Homo sapiens
augacugauuucuuuugguguucagagucaauauaauuuucuagcaccaucugaaaucgguuau

SEQ ID NO: 201 - RNA, 86 nt, Homo sapiens
acugcuaacgaaugcucugacuuuauugcacuacuguacuuuacagcuagcagugcaauaguauugucaaagcaucugaaagcagg

SEQ ID NO: 202 - RNA, 71 nt, Homo sapiens
gcgacuguaaacauccucgacuggaagcugugaagccacagaugggcuuucagucggauguuugcagcugc

SEQ ID NO: 203 - RNA, 88 nt, Homo sapiens
accaaguuucaguucauguaaacauccuacacucagcuguaauacauggauuggcugggagguggauguuuacuucagcugacuugga

SEQ ID NO: 204 - RNA, 72 nt, Homo sapiens
agauacuguaaacauccuacacucucagcuguggaaaguaagaaagcugggagaaggcuguuuacucuuucu

SEQ ID NO: 205 - RNA, 89 nt, Homo sapiens
accaugcuguaguguguguaaacauccuacacucucagcugugagcucaagguggcugggagaggguuguuuacuccuucugccaugga

SEQ ID NO: 206 - RNA, 70 nt, Homo sapiens
guuguuguaaacauccccgacuggaagcuguaagacacagcuaagcuuucagucagauguuugcugcuac

SEQ ID NO: 207 - RNA, 92 nt, Homo sapiens
gggcagucuuugcuacuguaaacauccuugacuggaagcuguaagguguucagaggagcuuucagucggauguuuacagcggcaggcugcca

SEQ ID NO: 208 - RNA, 68 nt, Homo sapiens
gaauggaagaagaaggcggucggucugcgggagccaggccgcagagccauccgccuucuguccauguc

SEQ ID NO: 209 - RNA, 85 nt, Homo sapiens
gguggucgagggaaucugagaaggcgcacaagguuuguguccaauacaguccacaccuugcgcuacucaggucugcucgugcccu

SEQ ID NO: 210 - RNA, 86 nt, Homo sapiens
uugguacuuggagagaggugguccguggcgcguucgcuuuauuuauggcgcacauuacacggucgaccucuuugcaguaucuaauc

SEQ ID NO: 211 - RNA, 83 nt, Homo sapiens
cugacuaugccuccccgcauccccuagggcauugguguaaagcuggagacccacugccccaggugcugcuggggguuguaguc

SEQ ID NO: 212 - RNA, 80 nt, Homo sapiens
gguaccugaagagagguuuucuggguuucuguuucuuuaaugaggacgaaacacaccugguuaaccucuuuuccaguauc

SEQ ID NO: 213 - RNA, 84 nt, Homo sapiens
gugguaccugaagagagguuuucuggguuucuguuucuuuauugaggacgaaacacaccugguuaaccucuuuuccaguaucaa

SEQ ID NO: 214 - RNA, 94 nt, Homo sapiens
cuuuggcgaucacugccucucugggccugugucuuaggcucugcaagaucaaccgagcaaagcacacggccugcagagaggcagcgcucugccc

SEQ ID NO: 215 - RNA, 94 nt, Homo sapiens
cggggcggccgcucucccuguccuccaggagcucacgugugccugccugugagcgccucgacgacagagccggcgccugccccagugucugcgc

SEQ ID NO: 216 - RNA, 98 nt, Homo sapiens
acccaaacccuaggucugcugacuccuaguccagggcucgugauggcuggugggcccugaacgaggggucuggaggccuggguuugaauaucgacagc

SEQ ID NO: 217 - RNA, 110 nt, Homo sapiens
ggccagcugugaguguuucuuuggcagugucuuagcugguuguugugagcaauaguaaggaagcaaucagcaaguauacugcccuagaagugcugcacguuguggggccc

SEQ ID NO: 218 - RNA, 84 nt, Homo sapiens
gugcucgguuuguaggcagugucauuagcugauuguacuguggugguuacaaucacuaacuccacugccaucaaaacaaggcac

SEQ ID NO: 219 - RNA, 77 nt, Homo sapiens
agucuaguuacuaggcaguguaguuagcugauugcuaauaguaccaaucacuaaccacacggccagguaaaaagauu

SEQ ID NO: 220 - RNA, 79 nt, Homo sapiens
aagguugcggugcaugugaugaagcaaaucaguaugaaugaauucaugauacuguaaacgcuuucugauguacuacuca

SEQ ID NO: 221 - RNA, 72 nt, Homo sapiens
ggagcuuaucagaaucuccagggguacuuuauaauuucaaaaagucccccaggugugauucugauuugcuuc

SEQ ID NO: 222 - RNA, 110 nt, Homo sapiens
ucccuggggaccccuggcagccccuccugaugauucuucuuccugagcacgcucaugaugagcaaacugagccucuaagaaguugacugaaggggcugcuuccccaagga

SEQ ID NO: 223 - RNA, 72 nt, Homo sapiens
uacaucggccauuauaauacaaccugauaaguguuauagcacuuaucagauuguauuguaauugucugugua

SEQ ID NO: 224 - RNA, 72 nt, Homo sapiens
acucggauggauauaauacaaccugcuaaguguccuagcacuuagcagguuguauuaucauuguccgugucu

SEQ ID NO: 225 - RNA, 70 nt, Homo sapiens
acacggacaaugauaauacaaccugcuaagugcuaggacacuuagcagguuguauuauauccauccgagu

SEQ ID NO: 226 - RNA, 66 nt, Homo sapiens
agggcuccugacuccagguccuguguguuaccuagaaauagcacuggacuuggagucagaaggccu

SEQ ID NO: 227 - RNA, 81 nt, Homo sapiens
ggaggccaucacuggacuuggagucagaagaguggagucgggucagacuucaacucugacuuugaagguggugagugccuc

SEQ ID NO: 228 - RNA, 98 nt, Homo sapiens
gaaugguuacaaggagagaacacuggacuuggagucagaaaacuuucauccaagucauucccugcucuaagucccauuucuguuccaugagauuguuu

SEQ ID NO: 229 - RNA, 54 nt, Homo sapiens
acuguuucuguccuuguucuuguuguuauuacuggacuuggagucagaaacagg

SEQ ID NO: 230 - RNA, 41 nt, Homo sapiens
cacugggcuuggagucagaagaccuggcuccagcccagcuc

SEQ ID NO: 231 - RNA, 76 nt, Homo sapiens
gggagcacuggacuaggagucagaagguggaguucugggugcuguuuucccacucuugggcccugggcauguucug

SEQ ID NO: 232 - RNA, 151 nt, Homo sapiens
ggguuggaaagcuguagguguggaggggcauggauacgggggccaugagggugggguccaggcuggaccaggccugcccugagucccccagcaggugcuccaggcuggcucacacccucugccucucucucuuccuuccuggccccaaccc

SEQ ID NO: 233 - RNA, 85 nt, Homo sapiens
gcacauuguaggccucauuaaauguuuguugaaugaaaaaaugaaucaucaacagacauuaauugggcgccugcucugugaucuc

SEQ ID NO: 234 - RNA, 90 nt, Homo sapiens
gagagaagcacuggacuuagggucagaaggccugagucucucugcugcagaugggcucucugucccugagccaagcuuuguccucccugg

SEQ ID NO: 235 - RNA, 94 nt, Homo sapiens
auaaaggaaguuaggcugaggggcagagagcgagacuuuucuauuuuccaaaagcucggucugaggccccucagucuugcuuccuaacccgcgc

SEQ ID NO: 236 - RNA, 98 nt, Homo sapiens
cgaggggauacagcagcaauucauguuuugaaguguucuaaaugguucaaaacgugaggcgcugcuauacccccucguggggaagguagaaggugggg

SEQ ID NO: 237 - RNA, 87 nt, Homo sapiens
gaaagcgcuuuggaaugacacgaucacucccguugagugggcacccgagaagccaucgggaaugucguguccgcccagugcucuuuc

SEQ ID NO: 238 - RNA, 91 nt, Homo sapiens
aagcugcuuaguguccuuagagucuccagaggcaucccuaacccagaaucuuuugacuguccucuggagagaaaggcaguaggucuguacc

SEQ ID NO: 239 - RNA, 72 nt, Homo sapiens
cggccccuuuguuaagggucucagcuccagggaacuuuaaaacccugagacccuaaccuuaaaggugcugca

SEQ ID NO: 240 - RNA, 55 nt, Homo sapiens
ccggauccgagucacggcaccaaauuucaugcguguccgugugaagagaccacca

SEQ ID NO: 241 - RNA, 72 nt, Homo sapiens
cuugggaauggcaaggaaaccguuaccauuacugaguuuaguaaugguaaugguucucuugcuauacccaga

SEQ ID NO: 242 - RNA, 60 nt, Homo sapiens
ucggcuaaggaaguccugugcucaguuuuguagcaucaaaacuaggauuucucuuguuac

SEQ ID NO: 243 - RNA, 115 nt, Homo sapiens
ucuguuuaucaccagauccuagaacccuaucaauauugucucugcuguguaaauaguucugaguagugcaauauugcuuauaggguuuugguguuuggaaagaacaaugggcagg

SEQ ID NO: 244 - RNA, 96 nt, Homo sapiens
ucccuggcgugaggguaugugccuuuggacuacaucguggaagccagcaccaugcaguccaugggcauauacacuugccucaaggccuaugucauc

SEQ ID NO: 245 - RNA, 90 nt, Homo sapiens
cugugggcugggccagggagcagcuggugggugggaaguaagaucugaccuggacuccaucccacccacccccuguuuccuggcccacag

SEQ ID NO: 246 - RNA, 75 nt, Homo sapiens
aggcuggcgugggcugagggcaggaggccuguggccggucccaggccuccugcuuccugggcucaggcucgguuu

SEQ ID NO: 247 - RNA, 71 nt, Homo sapiens
guugggggcuggggugcccacuccgcaaguuaucacugagcgacuuccggucugugagccccguccuccgc

SEQ ID NO: 248 - RNA, 60 nt, Homo sapiens
gagcaggcgaggcugggcugaacccgugggugaggagugcagcccagcugaggccucugc

SEQ ID NO: 249 - RNA, 74 nt, Homo sapiens
ccugcaggaggcagugggcgagcaggcggggcagcccaaugccaugggccugaucucaccgcugccuccuuccc

SEQ ID NO: 250 - RNA, 76 nt, Homo sapiens
gagggagcuguagagcagggagcaggaagcuguguguguccagcccugaccuguccuguucugcccccagccccuc

SEQ ID NO: 251 - RNA, 80 nt, Homo sapiens
ggagaaaggaguggaccgaggaaggaaggaaggcaaggcugucuguccauccguccgucuguccaccuaccugucagucc

SEQ ID NO: 252 - RNA, 84 nt, Homo sapiens
uugguacuuggagagugguuaucccuguccuguucguuuugcucaugucgaaucguacagggucauccacuuuuucaguaucaa

SEQ ID NO: 253 - RNA, 112 nt, Homo sapiens
ccaccccgguccugcucccgccccagcagcacacugugguuuguacggcacuguggccacguccaaaccacacugugguguuagagcgagggugggggaggcaccgccgagg

SEQ ID NO: 254 - RNA, 100 nt, Homo sapiens
agcucagggcggcugcgcagagggcuggacucagcggcggagcuggcugcuggccucaguucugccucuguccagguccuugugacccgcccgcucuccu

SEQ ID NO: 255 - RNA, 120 nt, Homo sapiens
gauccagggaacccuagagcagggggauggcagagcaaaauucauggccuacagcugccucuugccaaacugcacuggauuuugugucucccauuccccagagcugucugaggugcuuug

SEQ ID NO: 256 - RNA, 71 nt, Homo sapiens
ugcccuagcagcgggaacaguucugcagugagcgaucggugcucugggguauuguuuccgcugccagggua

SEQ ID NO: 257 - RNA, 83 nt, Homo sapiens
gcugcuguugggagacccuggucugcacucuaucuguauucuuacugaagggagugcagggcaggguuucccauacagagggc

SEQ ID NO: 258 - RNA, 84 nt, Homo sapiens
gaugcacccagugggggagccaggaaguauugauguuucugccaguuuagcgucaacacuugcugguuuccucucuggagcauc

SEQ ID NO: 259 - RNA, 119 nt, Homo sapiens
ccaugaggagcuggcagugggauggccuggggguaggagcguggcuucuggagcuagaccacauggguucagaucccagcggugccucuaacuggccacaggaccuugggcagucagcu

SEQ ID NO: 260 - RNA, 91 nt, Homo sapiens
cgacuugcuuucucuccuccaugccuugaguguaggaccguuggcaucuuaauuacccucccacacccaaggcuugcaaaaaagcgagccu

SEQ ID NO: 261 - RNA, 97 nt, Homo sapiens
cagaucucagacaucucggggaucaucaugucacgagauaccagugugcacuugugacagauugauaacugaaaggucugggagccacucaucuuca

SEQ ID NO: 262 - RNA, 65 nt, Homo sapiens
gcugaacucuagccugagcaacagagugagauggucuuguuuuguugcccaggcuggaguccagu

SEQ ID NO: 263 - RNA, 97 nt, Homo sapiens
uagggugaccagccauuaugguuugccugggacugaggaauuugcugggauaugucaguuccaggccaaccaggcugguuggucucccugaagcaac

SEQ ID NO: 264 - RNA, 85 nt, Homo sapiens
aggguagagggaugagggggaaaguucuauaguccuguaauuagaucucaggacuauagaacuuucccccucaucccucugcccu

SEQ ID NO: 265 - RNA, 97 nt, Homo sapiens
ucccuuucccaggggaggggcuggguuuacguugggagaacuuuuacggugaaccaggagguucucccaacguaagcccagccccuccccucugccu

SEQ ID NO: 266 - RNA, 67 nt, Homo sapiens
ccugcaggcagaaguggggcugacagggcagaggguugcgcccccucaccaucccuucugccugcag

SEQ ID NO: 267 - RNA, 98 nt, Homo sapiens
acgaauggcuaugcacugcacaacccuaggagagggugccauucacauagacuauaauugaauggcgccacuaggguugugcagugcacaaccuacac

SEQ ID NO: 268 - RNA, 61 nt, Homo sapiens
ugggcuaagggagaugauuggguagaaaguauuauucuauucauuugccucccagccuaca

SEQ ID NO: 269 - RNA, 118 nt, Homo sapiens
gcaggugaacuggcaggccaggaagaggaggaagcccuggaggggcuggaggugauggauguuuuccuccgguucucagggcuccaccucuuucgggccguagagccagggcuggugc

SEQ ID NO: 270 - RNA, 89 nt, Homo sapiens
augcaucgggauaguccgaguaacgucggggcauuccggauaggccgagaaaguguugugggaagaaaguuagauuuacgccgaugaau

SEQ ID NO: 271 - RNA, 88 nt, Homo sapiens
aacugcccucaaggagcuuacaaucuagcuggggguaaaugacuugcacaugaacacaacuagacugugagcuucuagagggcaggga

SEQ ID NO: 272 - RNA, 98 nt, Homo sapiens
uugggcaaggugcggggcuagggcuaacagcagucuuacugaagguuuccuggaaaccacgcacaugcuguugccacuaaccucaaccuuacucgguc

SEQ ID NO: 273 - RNA, 118 nt, Homo sapiens
gccuuggugcugauuccugggcucugaccugagaccucuggguucugagcugugauguugcucucgagcugggaucuccggggucuugguucagggccggggccucuggguuccaagc

SEQ ID NO: 274 - RNA, 89 nt, Homo sapiens
cgggguugguuguuaucuuugguuaucuagcuguaugagugguguggagucuucauaaagcuagauaaccgaaaguaaaaauaacccca

SEQ ID NO: 275 - RNA, 87 nt, Homo sapiens
ggaagcgaguuguuaucuuugguuaucuagcuguaugaguguauuggucuucauaaagcuagauaaccgaaaguaaaaacuccuuca

SEQ ID NO: 276 - RNA, 90 nt, Homo sapiens
ggaggcccguuucucucuuugguuaucuagcuguaugagugccacagagccgucauaaagcuagauaaccgaaaguagaaaugauucuca

SEQ ID NO: 277 - RNA, 81 nt, Homo sapiens
cccauuggcauaaacccguagauccgaucuuguggugaaguggaccgcacaagcucgcuucuaugggucugugucagugug

SEQ ID NO: 278 - RNA, 70 nt, Homo sapiens
ggcacccacccguagaaccgaccuugcggggccuucgccgcacacaagcucgugucuguggguccguguc

SEQ ID NO: 279 - RNA, 68 nt, Homo sapiens
ggguacaggugcacaacgugcagguuuguuacauauguauacauaugccauguuggugugcugcaccc

SEQ ID NO: 280 - RNA, 58 nt, Homo sapiens
ucgaggcuagagucacgcuuggguaucggcuauugccugagugugcuagaguccucga

SEQ ID NO: 281 - RNA, 78 nt, Homo sapiens
gagucacaguggacuuggagucaggaggccugagguccuugaagaccucccugaccugcucugguccacugugugcuc

SEQ ID NO: 282 - RNA, 66 nt, Homo sapiens
cuuugccgagacuagagucacauccugacacaacucuuguccuggugugcuagaguacucgaagag

SEQ ID NO: 283 - RNA, 102 nt, Homo sapiens
uuauacuuuaaguuuuaggguacaugugcacaacgugcagguuaguuacauauguauacuugugccauguuggugugcugcacccauuaacuugucguuuaa

SEQ ID NO: 284 - RNA, 114 nt, Homo sapiens
uccuacccgggucggaguuagcucaagcgguuaccuccucaugccggacuuucuaucuguccaucucugugcugggguucgagacccgcgggugcuuacugacccuuuuaugca

SEQ ID NO: 285 - RNA, 114 nt, Homo sapiens; identical to SEQ ID NO: 284

SEQ ID NO: 286 - RNA, 85 nt, Homo sapiens
auugaucaucgacacuucgaacgcaauugcagcccggguuccucccagggcuuugccugucugagcgucgcuugccgaucaguag

SEQ ID NO: 287 - RNA, 60 nt, Homo sapiens
uguuugcucaccugucauuucagcuuugugacaagccugacaugaaaaggggagagggca

SEQ ID NO: 288 - RNA, 103 nt, Homo sapiens
cugccaaagagcagcaagaugagcugguuugauggggagccaucccuugaugaggagaacccuucccacucucacucagccucacccagcugcccugaggcag

SEQ ID NO: 289 - RNA, 68 nt, Homo sapiens
gugacucugggaggugucggacgagcccccugaggucugucugugggcaacgccucccaaagugucau

SEQ ID NO: 290 - RNA, 82 nt, Homo sapiens
gcucagaagugaugaauugaucagauagacgaggccgggcuuguccccggccacugauuaucgaggcgauucugaucugggc

SEQ ID NO: 291 - RNA, 94 nt, Homo sapiens
guaggccugggucuuggauaaacugaaagcugagcgugaacgugguaucaccauuaauaccuccuuguggaaauuugagaacagcaagugcuac

SEQ ID NO: 292 - RNA, 74 nt, Homo sapiens
acccuacuccugguaccaauuuacuguauuagucuguuuucaugcugcugauagagacauacccaggagugggu

SEQ ID NO: 293 - RNA, 55 nt, Homo sapiens
gacgugagggggugcuacauacagcagcuguguguaguaugugccuuucucuguu

SEQ ID NO: 294 - RNA, 84 nt, Homo sapiens
ugguuggccgguccagcgccuccaggaagucgaagcugccgcaguggcgcuuguugaaaggcgcgggcucagcaggccgagcca

SEQ ID NO: 295 - RNA, 130 nt, Homo sapiens
uaggaauucuggaccaggcuuaaaagacugggaugaggcugguccgaagguagugaguuaucuccauugauaguucagucuguaacagaucaaacuccuuguucuacucuuuuuuuuuuuuuuagacaga

SEQ ID NO: 296 - RNA, 103 nt, Homo sapiens
ugggcugguccgagugcagugguguuuacaaguauuugauuauaacuaguuacagauuucuuuguuuccuucuccacucccacugccucacuugacuggccua

SEQ ID NO: 297 - RNA, 120 nt, Homo sapiens
uggugguauugguaguguuccuguuggccgaguggagacugguguucucaaaccugguaugguggucaccuuugcuccagucaacguuacaacagaaguaaaaucugucgaaaugcacca

SEQ ID NO: 298 - RNA, 89 nt, Homo sapiens
cuaauggauaaggcauuggccuccuaagccagggauuguggguucgagucccaucugggguggccugugacuuuuguccuuuuuucccc

SEQ ID NO: 299 - RNA, 75 nt, Homo sapiens
aguugguccgaguguuguggguuauuguuaaguugauuuaacauugucuccccccacaaccgcgcuugacuagcu

(19) United States
(12) Patent Application Publication
(10) Pub. No.: US 2016/0145628 A1
(43) Pub. Date: May 26, 2016

(54) NANOCARRIER SYSTEM FOR MICRORNAS AND USES THEREOF

(71) Applicants: RAMOT AT TEL-AVIV UNIVERSITY LTD., Tel Aviv (IL); ROSETTA GENOMICS LTD., Rehovot (IL)

(72) Inventors: Noga Yerushalmi, Nes Ziona (IL); Sharon Kredo-Russo, Karmey-Yosef (IL); Gila Lithwick Yanai, Modiin (IL); Ronit Satchi-Fainaro, Tel Aviv (IL); Paula Ofek, Givatayim (IL)

(21) Appl. No.: 14/899,183

(22) PCT Filed: Jun. 18, 2014
(86) PCT No.: PCT/IB2014/062389

§ 371 Date: Dec. 17, 2015

Related U.S. Application Data
(60) Provisional application No. 61/836,204, filed on Jun. 18, 2013.

(51) Int. Cl. C12N 15/113; A61K 31/713; A61K 47/48

(52) U.S. Cl. CPC C12N 15/1138; A61K 47/48215; A61K 31/713; C12N 2310/141; C12N 2310/351

(57) ABSTRACT

Described herein are novel polyglycerol-amine polymeric nanocarriers in complex with microRNAs and their uses in the treatment of cancer, in particular glioblastoma. Delivery of the polymeric nanocarriers in complex with microRNAs in cell lines and in vivo inhibited cell proliferation, cell cycle progression, cell migration and tumor growth.

### RELATED APPLICATIONS

This application claims priority from, and the benefit of, U.S. Application No. 61/836,204, filed Jun. 18, 2013, the contents of which are incorporated herein by reference.

### FIELD OF THE INVENTION

The present invention relates to a novel system for the transport of microRNAs and its uses in therapy.

### BACKGROUND OF THE INVENTION

MicroRNAs (miRNAs, miRs) are an important class of regulatory RNAs which has profound impact on a wide array of biological processes. These small (typically 18-24 nucleotides long) non-coding RNA molecules can modulate protein expression patterns by e.g. promoting RNA degradation, inhibiting mRNA translation, as well as affecting gene transcription. MiRs play pivotal roles in diverse processes such as development and differentiation, control of cell proliferation, stress response and metabolism. The expression of many miRs was found to be altered in numerous types of human cancer, and strong evidence has suggested a causative role in tumor progression. Cancer-associated changes in miR expression patterns can be brought about by various genetic and epigenetic mechanisms. Most notably, a number of transcription factors whose activity is altered in cancer cells, including c-myc and E2F, were found to regulate the RNA polymerase II-dependent transcription of precursors of particular miRs. Hence, the oncogenic effects of these transcription factors may be mediated not only by modulation of protein-coding mRNA levels but also by specific changes in miR expression. In counterpart, miRs have also been shown to be capable of regulating cell proliferation and apoptosis, and thus having potential therapeutic effects in cancer.

Glioblastoma multiforme (GBM) is the most common form of primary brain tumor. It is one of the most aggressive forms of human cancer. Without treatment, the median survival is approximately 3 months. The most common chemotherapy used in the treatment of GBM is temozolomide (TMZ). Together with radiotherapy and surgery, the median survival of GBM patients is approximately 14 months. Due to GBM's diffusive and invasive nature to the surrounding normal brain tissue, complete removal of the tumor is impossible by surgery, resulting in very high recurrence rate of 95% from residual tumor volume. Management options for recurrent GBM include a second cycle of surgery, radiation and/or chemotherapy.

### SUMMARY OF THE INVENTION

In view of the shortcomings in current treatment pathways of tumors in general and of glioblastoma in particular, there is a widely recognized need for novel agents and novel targeting pathways for treating tumors.

The present inventors have utilized a cationic carrier system, which can strongly improve microRNA stability, intracellular trafficking as well as miRNA's silencing efficacy, and which further exhibits accumulation in tumor and hence can be used in cancer therapy.

Thus, in a first aspect, the present invention provides a system comprising at least one nanocarrier and at least one nucleic acid molecule, said nanocarrier being a compound having a structure according to formula (I),

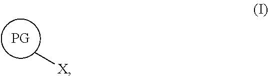

wherein PG denotes a linear or branched polyglycerol core, and X is covalently bound to a carbon atom of the polyglycerol core and is independently selected at each instance from the group consisting of (a) —NR¹R², (b) —OC(═O)—NR³R⁴, (c) —NH—C(O)—CH₂CH₂—S—S—S—[CH₂CH₂O]_{q}—Y and (d) —CH(NH₂)—CH₂—NH—C(O)—CH₂CH₂—S—S—[CH₂CH₂O]_{q}—Y, wherein at each occurrence q is independently 20-50 and Y is independently H or CH₃,
wherein the polyglycerol core carries a plurality of groups of the type X,
R¹ is (i) H, (ii) linear or branched C₁-C₁₀-alkyl which may be substituted and/or interrupted by one or more oxygen, sulphur and/or nitrogen atoms, or by a group R³, or (iii) a group R³;
R² is (i) H, (ii) linear or branched C₁-C₁₀-alkyl which may be substituted and/or interrupted by one or more oxygen, sulphur and/or nitrogen atoms, or by a group R³, or (iii) R³;
R³ is —(CH₂CH₂NH)ₙ—H, wherein n is 1-100;
R⁴ is H or C₁-C₄-alkyl which may be substituted and/or interrupted by one or more oxygen, sulphur and/or nitrogen atoms;
and said nucleic acid molecule comprises a sequence denoted by any one of SEQ ID NO.1-145 and 152-299, or a sequence at least about 80% identical to any one of SEQ ID NO.1-145 and 152-299. In one particular embodiment, said nucleic acid is complementary to a sequence denoted by any one of SEQ ID NO.1-145, or to a sequence at least about 80% identical to any one of SEQ ID NO.1-145. In another particular embodiment, said nucleic acid molecule comprises a sequence denoted by SEQ ID NO.63, or a variant thereof.

In a further particular embodiment of the system of the invention, about 10% of the X groups have a structure selected from —NH—C(O)—CH₂CH₂—S—S—[CH₂CH₂O]ₘ—Y and —CH(NH₂)—CH₂—NH—C(O)—CH₂CH₂—S—S—[CH₂CH₂O]_{q}—Y, wherein q is on average 20-50 and Y is H or CH₃.

In some embodiments, q is independently at each occurrence 40-50; in some embodiments q is at each occurrence 44-45. In some embodiments, the nanocarrier compound further comprises a fluorescein label. In some embodiments the fluorescein label is attached to the PG core via a bond formed between an amine moiety pendant from the PG core and an isothiocyanate unit covalently attached to the fluorescein. In some embodiments not more than ten, not more than nine, not more than eight, not more than seven, not more than six, not more than five, not more than four, not more than three, not more than two or fluorescein moieties are attached to the PG core. In some embodiments a single fluorescein moiety is attached to the PG core.

In one particular embodiment said nucleic acid is to be carried by or bound to said nanocarrier in any one of a covalent, ionic or complexed manner.

In another aspect there is provided in accordance with an embodiment of the present invention a pharmaceutical composition comprising the system described herein.

In a further aspect, there is provided a method of treating cancer, said method comprising administering a therapeutically effective amount of the system according to the invention, or a composition comprising same, to a subject in need thereof. In one embodiment, said cancer is brain cancer.

In another aspect, there is provided in accordance with an embodiment of the invention a system for use in the treatment of cancer, such as, for example, brain cancer.

In a further aspect, there is provided a method of inhibiting or mimicking microRNA function in the cell, said method comprising contacting said cell with the system according to the invention, or with a composition comprising the same.

In another aspect, there is provided the use of the system described herein for the preparation of a pharmaceutical composition or medicament for treating cancer such as, for example, brain cancer.

In another aspect there is provided a kit, said kit comprising the system according to the invention, or a composition comprising the same, means for administering said system or said composition to a patient in need. Said kit optionally comprising instructions of dosage and/or administration of said system or composition. In one embodiment, said kit is intended for use in the treatment of cancer, such as, for example, brain cancer.

### BRIEF DESCRIPTION OF THE DRAWINGS

FIG. 1 shows the chemical structure of hyperbranched polymer of polyglycerol-amine PG-NH₂ [Ofek et al. (2010) FASEB Journal, 24(9), p. 3122-34, incorporated herein by reference].

FIG. 2 shows a photograph of an electrophoretic mobility shift assay (EMSA) of PG-NH₂-miR polyplexes, with increasing amounts of PG-NH₂. PG-NH₂-miR-34a was loaded on the first four wells, and PG-NH₂—NC was loaded on the last four wells.

FIG. 3 shows a graph of growth inhibition of human glioblastoma cells in vitro by PG-NH₂-miR-34a polyplex. The x-axis presents the result of proliferation assays in three cell lines, U87-MG, A172 and T98G: the left-most column for each cell line represents treatment with PG-NH₂-miR-34a; the column in the center for each cell line represents treatment with PG-NH₂—NC; and the right-most column for each cell line represents negative control (no treatment). The y-axis represents cell proliferation (% of control).

FIGS. 4A-4E show inhibition of human glioblastoma cells migration towards FBS by the PG-NH₂-miR34a polyplex.

FIG. 4A shows photographs of cell migration in U87-MG cells. Top right panel, no FBS. Top left panel, FBS-treated. Lower left panel, treated with FBS and transfected with PG-NH₂—NC. Lower right panel, treated with FBS and transfected with PG-NH₂-miR-34a.

FIG. 4B shows a graph of % migrating cells (y-axis) in U87-MG cells treated with control (left-most column), PG-NH₂—NC (center column) or PG-NH₂-miR-34a (right-most column).

FIG. 4C shows photographs of cell migration in A172 cells. Top right panel, no FBS. Top left panel, FBS-treated. Lower left panel, treated with FBS and transfected with PG-NH₂—NC. Lower right panel, treated with FBS and transfected with PG-NH₂-miR-34a.

FIG. 4D shows a graph of % migrating cells (y-axis) in A172 cells treated with control (left-most column), PG-NH₂—NC (center column) or PG-NH₂-miR-34a (right-most column). *** p value < or =0.01 related to control and to negative control (NC) miR.

FIG. 4E shows human umbilical vein endothelial cells (HUVEC) migration towards conditioned media (C.M.) from A172 cells transfected with control, PG-NH₂—NC, or PG-NH₂-miR34a polyplex.

FIG. 5 shows a graphical representation of the results from fluorescence-activated cell sorting (FACS) analysis of U-87 MG cells untreated (control), transfected with PG-NH₂-miR34a or PG-NH₂—NC.

FIGS. 6A-6B show internalization of PG-NH₂-miR-34a polyplex into U-87 MG cells, accompanied by increased expression level of hsa-miR-34a decreased expression level of hsa-miR-34a target genes c-Met and Notch1.

FIG. 6A shows a graph (left) representing hsa-miR-34a expression in U-87 MG cells untreated (control), transfected with PG-NH₂—NC or PG-NH₂-miR34a. The graph on the right represents c-Met expression in U-87 MG cells untreated (control), transfected with PG-NH₂—NC or PG-NH₂-miR34a.

FIG. 6B shows a Western blot of c-Met, Notch1 and β-actin proteins in U-87 MG cells untreated (control), transfected with PG-NH₂—NC or transfected with PG-NH₂-miR34a.

FIGS. 7A-7B show tumor volume and survival in U-87 MG glioblastoma tumor model in vivo in SCID mice treated with PG-NH2-miR-34a.

FIG. 7A shows a graph representing tumor volume (y-axis, mm³) in U-87 MG glioblastoma tumor model SCID mice treated with PG-NH2-miR-34a (n=3), PG-NH2-NC-miR (NC-miR, n=4), or saline (n=4), for a total period of 50 days (x-axis). Data represents mean±s.e.m.

FIG. 7B shows a graph representing a Kaplan-Meier analysis for survival (y-axis) of U-87 MG glioblastoma tumor model SCID mice following three consecutive treatments (on days 0, 3 and 6) with PG-NH2-miR-34a (n=3), PG-NH2-NC-miR (NC-miR, n=4), or saline (n=4), in time (x-axis, days).

FIGS. 8A-8C show a comparison of microRNA expression in samples from human GBM obtained from long-term survivors (LTS) patients versus short-term survivors (STS) patients. The data are shown in normalized fluorescence units, as measured by microarray. Each blue “+” signifies the median expression of a microRNA in both groups. In gray are control probes or microRNA probes with median expression less than 300 in both groups.

FIG. 8A shows a comparison of the microRNA expression in all LTS samples (n=12, y-axis) versus all STS samples (n=10, x-axis) is shown. Encircled in red and labelled are 21 microRNAs with p-value ≦0.05 and fold-change ≧1.5, which are hsa-miR-212-3p, hsa-miR-1290, hsa-miR-18b-5p, hsa-miR-503-5p, hsa-miR-4732-5p, hsa-miR-30a-3p, hsa-miR-4690-5p, hsa-miR-18a-5p, hsa-miR-130b-3p, hsa-miR-10b-5p, MID-01141, MID-01140, hsa-miR-138-5p, hsa-miR-124-3p, MID-19433, hsa-miR-195-5p, MID-16318, hsa-miR-30c-5p, hsa-miR-4664-5p, hsa-miR-30a-5p, and hsa-miR-26b-5p. Encircled in pink are 34 microRNA with p-value ≦0.05 and fold-change <1.5.

FIG. 8B shows a comparison of 1^{st} surgery LTS samples (n=6, y-axis) versus all STS samples (n=10, x-axis). Encircled in red and labelled are 14 microRNAs with p-value ≦0.05 and fold-change ≧2.5, which are hsa-miR-9-5p, hsa-miR-374b-5p, hsa-miR-124-3p, hsa-miR-221-3p, hsa-miR-222-3p, hsa-miR-34a-5p, hsa-miR-210-3p, hsa-miR-10b-5p, hsa-miR-155-5p, hsa-miR-34c-5p, hsa-miR-1290, hsa-miR-34b-5p, MID-01141 and MID-01140. Encircled in pink are 94 microRNAs with p-value ≦0.05 and fold-change <2.5.

FIG. 8C shows a comparison of 1^{st} surgery LTS samples (n=6, y-axis) with STS samples who were either treated or who had very short survival times (n=6, x-axis). Encircled in red and labelled are 10 microRNAs with p-value ≦0.05 and fold-change ≧3, which are hsa-miR-221-3p, hsa-miR-222-3p, hsa-miR-34a-5p, hsa-miR-210-3p, hsa-miR-155-5p, hsa-miR-34c-5p, hsa-miR-1290, hsa-miR-34b-5p, MID-01141 and MID-01140. Encircled in pink are 97 microRNAs with p-value ≦0.05 and fold-change <3.

FIG. 9 shows nanocarrier FS-157.

FIG. 10 shows an electrophoresis mobility-shift assay of the PG-NH₂-derivative FS-157 combined with of hsa-miR-34a. M=molar ratio; N/P=N/P ratio.

FIGS. 11A-11B show the effect of PG-NH₂-miR-34a and FS-157 conjugated to hsa-miR-34a transfected in HeLa cells on a psi-CHECK reporter.

FIG. 11A shows a graph representing Renilla luciferase reporter activity of PG-NH₂-miR-34a and FS-157 conjugated to hsa-miR-34a. Activity of the miR-34 luciferase reporter (miR-34a y-axis) is presented for PG-NH₂-miR-34a and FS-157 conjugated to hsa-miR-34a transfected at the indicated N/P ratios.

FIG. 11B shows a graph representing viability (% viab., y-axis) of cells transfected with PG-NH₂-miR-34a or FS-157 conjugated to hsa-miR-34a, at the indicated N/P ratios.

FIGS. 12A-12F show intra-cellular trafficking and co-localization of PG-NH₂-Cy5-siRNA or FS-157-Cy5-siRNA with the endosomal marker EEA1 or with the lysosomal marker LAMP1 in U87 MG cells at 3 hours (top row), 5 hours (middle row) or 24 hours (bottom row) after transfection.

FIG. 12A shows a confocal microscopy photograph of intra-cellular trafficking and co-localization of PG-NH₂-Cy5-siRNA (left column) and the endosomal marker EEA1 (middle column). The right-most column shows the merge of the two.

FIG. 12B shows a confocal microscopy photograph of intra-cellular trafficking and co-localization of PG-NH₂-Cy5-siRNA (left column) and the lysosomal marker LAMP1 (middle column). The right-most column shows the merge of the two.

FIG. 12C shows a confocal microscopy photograph of intra-cellular trafficking and co-localization of FS-157-Cy5-siRNA (left column) and the endosomal marker EEA1 (middle column). The right-most column shows the merge of the two.

FIG. 12D shows a confocal microscopy photograph of intra-cellular trafficking and co-localization of FS-157-Cy5-siRNA (left column) and the lysosomal marker LAMP1 (middle column). The right-most column shows the merge of the two.

FIG. 12E is a graph representing the quantification of co-localization of PG-NH₂-Cy5-siRNA with the endosomal marker EEA1 or with the lysosomal marker LAMP1 in U87 MG cells. Y-axis represents the % co-localization of the polyplex with each marker at the indicated time-points (3, 5 or 24 hours).

FIG. 12F is a graph representing the quantification of co-localization of FS-157-Cy5-siRNA with the endosomal marker EEA1 or with the lysosomal marker LAMP1 in U87 MG cells. Y-axis represents the % co-localization of the polyplex with each marker at the indicated time-points (3, 5 or 24 hours).

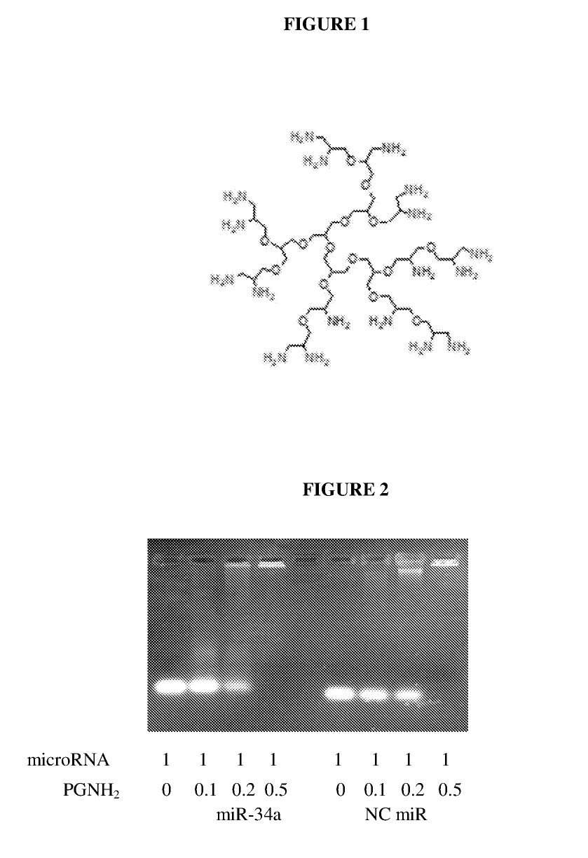

### DETAILED DESCRIPTION OF THE INVENTION

MicroRNAs play an important role in cancer in general and in glioblastoma in particular, having a broad therapeutic potential. Nonetheless, their delivery to the brain could be challenging. Thus, it is desirable to design an efficient delivery system which allows a therapeutically active agent to reach the target tissue, enhance cell entrance, and enable endosomal escape, so as to make these molecules bio-available in the cytoplasm.

A delivery system that enables high activity of microRNA in a cell with low cytotoxicity, and which is proven as biocompatible systemically in vivo is the holy grail for microRNA delivery.

Thus, there is a need for novel and efficient delivery systems for use as therapeutic agents, particularly in the treatment of cancer.

The present inventors developed a novel polymeric delivery system in which a nucleic acid molecule that mimics or inhibits the sequence and activity of a microRNA (miR, miRNA) is encapsulated or is complexed in a cationic carrier system.

The novel polymeric delivery system described herein may carry a nucleic acid in the form of a duplex, in which said duplex comprises double-stranded RNA consisting of two segments of RNA held in a double helix by complementary base pairing. The two strands are oriented in an antiparallel fashion to one another. Alternatively, said duplex comprises the sequence of a microRNA hairpin, which may fold and form a double-stranded stem.

Alternatively, the novel polymeric delivery system encapsulates or is complexed with a single-stranded nucleic acid, said single stranded nucleic acid comprising an anti-microRNA molecule, which inhibits the activity of the endogenous microRNA.

In search for a methodology that would allow efficient utilization of miRs, the present inventors have devised and successfully prepared and utilized a cationic carrier system, which significantly improves the stability, intracellular trafficking, silencing efficacy, tumor accumulation and activity of the miR.

This methodology was demonstrated while using a polyglycerol (PG)-Amine, a water-soluble polyglycerol-based hyperbranched polymer that accumulates in the tumor environment due to the enhanced permeability and retention (EPR) effect. Without being bound by any particular theory, and as shown in the Examples below, PG-Amine complexation of an exemplary miR neutralizes its negative charge in a dose-dependent manner and significantly improves its cellular uptake.

Thus, in a first aspect of some embodiments of the present invention, there is provided a system comprising at least one nanocarrier and at least one nucleic acid molecule.

The system presented herein is capable of reaching and accumulating in the cells and/or in the tumor tissue selectively, and is characterized by in vivo bioavailability and by low toxicity.

In some embodiments, the nanocarrier is a cationic system as described in WO 2009/141170, the contents of which are incorporated herein in their entirety.

In some embodiments described herein, the system comprises a compound, also referred to as nanocarrier, having a structure according to formula (I),

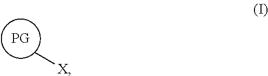

wherein PG denotes a linear or branched polyglycerol core, and X is covalently bound to a carbon atom of the polyglycerol core and is independently selected at each instance from the group consisting of (a) —NR¹R², (b) —OC(═O)—NR³R⁴, (c) —NH—C(O)—CH₂CH₂—S—S—[CH₂CH₂O]_{q}—Y and (d) —CH(NH₂)—CH₂—NH—C(O)—CH₂CH₂—S—S—[CH₂CH₂O]_{q}—Y, wherein q is on average 20-50 and Y is H or CH₃,
wherein the polyglycerol core carries a plurality of groups of the type X,
R¹ is (i) H, (ii) linear or branched C₁-C₁₀-alkyl which may be substituted and/or interrupted by one or more oxygen, sulphur and/or nitrogen atoms, or by a group R³, or (iii) a group R³;
R² is (i) H, (ii) linear or branched C₁-C₁₀-alkyl which may be substituted and/or interrupted by one or more oxygen, sulphur and/or nitrogen atoms, or by a group R³, or (iii) a group R³;
R³ is —(CH₂CH₂NH)ₙ—H, wherein n is 1-100; R⁴ is (i) H or (ii) C₁-C₄-alkyl which may be substituted and/or interrupted by one or more oxygen, sulphur and/or nitrogen atoms
and a nucleic acid molecule comprising a sequence denoted by any one of SEQ ID NO. 1-299, or a sequence at least about 80%, or at least about 85%, or at least about 90%, or at least about 95% identical to any one of SEQ ID NO.1-299.

In some embodiments described herein, the system comprises a compound of formula (I) as defined above, and the nucleic acid molecule comprises a sequence denoted by any one of SEQ ID NO.1-145, or a sequence at least about 80%, or at least about 85%, or at least about 90%, or at least about 95% identical to any one of SEQ ID NO.1-145.

In one particular embodiment said nucleic acid comprises a sequence denoted by any one of SEQ ID NO.123, SEQ ID NO.14, SEQ ID NO.117, SEQ ID NO.65, SEQ ID NO.70, SEQ ID NO.122, SEQ ID NO.32, SEQ ID NO.64, SEQ ID NO.63, SEQ ID NO.24, SEQ ID NO.108, SEQ ID NO.130, SEQ ID NO.131, SEQ ID NO.62, SEQ ID NO.15, SEQ ID NO.84 and SEQ ID NO.71, or a complement thereof, or a sequence at least about 80%, or at least about 85%, or at least about 90%, or at least about 95% identical to any one of SEQ ID NO.123, SEQ ID NO.14, SEQ ID NO.117, SEQ ID NO.65, SEQ ID NO.70, SEQ ID NO.122, SEQ ID NO.32, SEQ ID NO.64, SEQ ID NO.63, SEQ ID NO.24, SEQ ID NO.108, SEQ ID NO.130, SEQ ID NO.131, SEQ ID NO.62, SEQ ID NO.15, SEQ ID NO.84 and SEQ ID NO.71, or a complement thereof.

In one particular embodiment, said nucleic acid molecule comprises hsa-miR-34a-5p, denoted by SEQ ID NO.63, or a complement thereof, or a sequence at least about 80%, or at least about 85%, or at least about 90%, or at least about 95% identical to SEQ ID NO.63, or a complement thereof.

In some embodiments, q is independently at each occurrence 40-50; in some embodiments q is independently at each occurrence 44-45.

In some embodiments, X is selected from (a) and (b). In some embodiments X is selected from (c) and (d).

In some embodiments, the nanocarrier compound further comprises a fluorescein label. In some embodiments the fluorescein label is attached to the PG core via a bond formed between an amine moiety pendant from the PG core and an isothiocyanate unit covalently attached to the fluorescein. In some embodiments not more than ten, not more than nine, not more than eight, not more than seven, not more than six, not more than five, not more than four, not more than three, not more than two or fluorescein moieties are attached to the PG core. In some embodiments a single fluorescein moiety is attached to the PG core.

In one embodiment of the invention, the nanocarrier comprises a polyglycerol core in which at least 20%, at least 30%, at least 40%, at least 50%, particularly at least 60%, particularly at least 70%, particularly at least 80%, particularly at least 90%, particularly at least 95%, particularly at least 99%, particularly all of the free hydroxyl groups of the polyglycerol core are substituted by groups of the type X. The rate of substitution is also referred to as conversion. Thus, if a conversion of 100% is achieved during synthesis, the starting material polyglycerol of the formula PG-(OH)ₚ was reacted to PG-(X)ₘ with m=p. If, e.g., a product of the formula (X)ₘ—PG-(OH)_{q} with m=0.8*n and q=0.2*p is obtained, the conversion is 80%.

It is to be understood that when a free hydroxyl group which is substituted by a group of type X, the carbon atom which was previously bound to a hydroxyl group becomes bound to the group of type X instead of being bound to the hydroxyl group.

In some embodiments of the invention, the free hydroxyl groups of the polyglycerol core are substituted by groups of the type X to a degree such that the nanocarrier comprises at least 0.5 nitrogen atoms per free hydroxyl group remaining in the polyglycerol core (i.e., after substitution of at least a portion of the free hydroxyl groups). In some embodiments, the nanocarrier comprises at least 1 nitrogen atom per free hydroxyl group remaining in the polyglycerol core. In some embodiments, the nanocarrier comprises at least 2 nitrogen atoms per free hydroxyl group remaining in the polyglycerol core. In some embodiments, the nanocarrier comprises at least 5 nitrogen atoms per free hydroxyl group remaining in the polyglycerol core. In some embodiments, the nanocarrier comprises at least 10 nitrogen atoms per free hydroxyl group remaining in the polyglycerol core. In some embodiments, the nanocarrier comprises at least 20 nitrogen atoms per free hydroxyl group remaining in the polyglycerol core.

It is to be appreciated that the proportion of nitrogen atoms free hydroxyl groups in the nanocarrier will depend on both the proportion of groups of type X and the number of nitrogen atoms in each group of type X.

In some of the embodiments described herein, n is preferably 1 to 10. In some further embodiments, n is 5.

In some of the embodiments of the invention, R¹ is H. In such embodiments, —NR¹R² may be a primary or secondary amine group.

In some of the embodiments of the invention, R¹ and R² are each H, such that —NR¹R² is a primary amine group.

In some of the embodiments of the invention, R¹ is H and R² is not H, such that —NR¹R² is a secondary amine group.

In some of the embodiments of the invention, neither R¹ nor R² are H, such that —NR¹R² is a tertiary amine group.

In some of the embodiments of the invention, one or more of R¹, R², R³ or R⁴ is PEGylated.

In some of the embodiments of the invention, one or more X groups contain a fluorophore.

In some of the embodiments of the invention, the polyglycerol core may carry a plurality of groups of the type X, and between 1-20% of X group is PEGylated. Thus, a polyglycerol core carrying a plurality of groups of the type X may present 1%, 2%, 3%, 4%, 5%, 6%, 7%, 8%, 9%, 10%, 11%, 12%, 13%, 14%, 15%, 16%, 17%, 18%, 19% or 20% of type X groups containing PEG.

In one particular embodiment of the invention, the polyglycerol core carriers a plurality of groups of the type X, wherein a maximum of 10% of groups X contain PEG.

As provided herein, fluorophores suitable to be carried by the PG-nanocarrier of the invention are fluorophores at the 400/420-790/810 nm range. Examples of such fluorophores are fluorescein, Cy3 (1-{6-[(2,5-Dioxo-1-pyrrolidinyl)oxy]-6-oxohexyl}-2-[(1E,3E)-3-(1-{6-[(2,5-dioxo-1-pyrrolidinyl)oxy]-6-oxohexyl}-3,3-dimethyl-5-sulfo-1,3-dihydro-2H-indol-2-ylidene)-1-propen-1-yl]-3,3-dimethyl-3H-indolium-5-sulfonate) and Cy5 (1-{6-[(2,5-Dioxo-1-pyrrolidinyl)oxy]-6-oxohexyl}-2-[(1 E,3E,5E)-5-(1-{6-[(2,5-dioxo-1-pyrrolidinyl)oxy]-6-oxohexyl}-3,3-dimethyl-5-sulfo-1,3-dihydro-2H-indol-2-ylidene)-1,3-pentadien-1-yl]-3,3-dimethyl-3H-indolium-5-sulfonate).

In some of the embodiments, R¹ is an alkyl. In exemplary embodiments, the alkyl is methyl.

In some of the embodiments described herein, the linear or branched C₁-C₁₀-alkyl is substituted and/or interrupted by one or more nitrogen atoms. In some further embodiments, the linear or branched C₁-C₁₀-alkyl comprises an alkyl group substituted by an amine group, for example, a primary amine group (—NH₂), secondary amine group (—NH-alkyl) and/or a tertiary amine group (—N(alkyl)₂).

In some of the embodiments described herein, the C₁-C₁₀-alkyl is ethyl substituted by an amine group (e.g., at the 2-position of the ethyl), for example, a 2-(N,N-dialkylamino)ethyl group. In some embodiments, the C₁-C₁₀-alkyl is 2-(N,N-dimethylamino)ethyl.

In some of the embodiments described herein, R² is 2-(N,N-dimethylamino)ethyl.

In some of the embodiments described herein, the nanocarrier comprises a compound in which R¹ is a methyl residue and R² is 2-(N,N-dimethylamino)ethyl, such that an N,N,N′-trimethylethylenediamine residue is bound to the polyglycerol core structure via one of its nitrogen atoms.

In some embodiments, R¹ and R² cannot simultaneously be an ethyl residue.

In some embodiments of any of the aspects of the invention described herein, n is 1 to 10, particularly 2 to 8, particularly 3 to 6 and in particular 5.

As can be seen from formula (I) and the residue definitions given above, the nanocarrier comprised in the system of the invention has a polyglycerol (PG) based gene-transfection motif with core-shell architecture. As referred to herein, the gene-transfection motif is a positively-charged motif, as found in the nanocarrier polymer of the present invention. The outer shell may contain PEG moieties. The shells of such motifs can be tailored to contain amines with different numbers of cationic sites for mimicking the activity of polyamines. Since the nanocarriers are based on a PG structure, they provide appreciable clinical compliance.

In contrast to polyamines and other known compounds used as carriers, the nanocarriers comprised in the system as described herein carry charges at physiological pH only on their surface or shell (namely on nitrogen atoms located on the surface or being part of the shell), whereas the core is substantially not charged, being formed of short alkyl chains connected to each other via ether bridges. The polyglycerol core may be structured in a linear or branched manner. In one embodiment, the structure of the polyglycerol is at least partially branched.

The shell of the polyglycerol-based compounds may have a layered structure due to a repetitive nitrogen-containing motif. E.g., by use of a pentaethylenehexamine residue as shell (as is the case in polyglyceryl pentaethylenehexamine carbamate), a five-fold layered shell is achieved.

The polyglycerol base material can be obtained in a large (e.g., kilogram) scale which contains linear monohydroxy and terminal dihydroxy functionalities which can be modified selectively as linkers for diverse organic synthesis.

The polyglycerol core of the nanocarriers comprised in the system as described herein is biocompatible. Generally, by introducing nitrogen-containing shell motifs, the cell toxicity of the nanocarrier is raised, in addition to transfection efficacy. In the nanocarriers as described herein, a balance between toxicity and transfection efficacy is achieved.

Specific, symmetric polyglycerol dendrimers are an example of polyglycerol which can be used for the polyglycerol core of the nanocarrier comprised in the system of the invention. These dendrimers are symmetric. They are generated from smaller molecules by repeated reaction steps, wherein each step results in a higher degree of branching compared to the preceding step. At the end of the branches, functional groups are located which are the starting point for further branchings. Thus, with each reaction step, the number of monomeric end groups increases exponentially, leading to a hemicircular tree structure.

In this context, the term “polyglycerol” as used herein includes any substance which contains at least two etherically linked glycerol units in its molecule and wherein said molecule is characterized by a branched structure. According to the present invention, the term “glycerol unit” does not only relate to glycerol itself but also includes any subunits which are based on glycerol, such as for example:

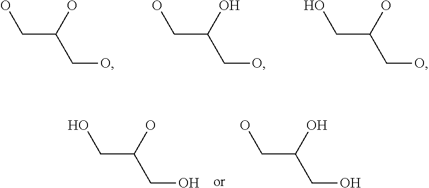

wherein “bare” oxygen atoms can have various chemical groups attached thereto.

Preferably, the polyglycerol includes three or more, preferably ten or more, and particularly 15 or more of said glycerol units. The polyglycerol structure can be obtained, e.g., by a perfect dendrimer synthesis, a hyperbranched polymer synthesis or a combination of both using methodologies that would be readily recognized by a person skilled in the art.

In some of the embodiments described herein, when X in the Formulae described herein is —OC(═O)—NR³R⁴, n is 5 and R⁴ is H, the nanocarrier comprised in the system of the invention would be polyglyceryl pentaethylenehexamine carbamate.

In some of the embodiments described herein, the entity carrier bears at least one functional group of the general formula —OC(═O)—NR³R⁴, wherein residues R³ and R⁴ are as defined hereinabove, and the functional group is cleaved from the polyglycerol core of the nanocarrier once the nanocarrier is located within its target cell. This cleavage results in an even better biocompatibility of the nanocarrier comprised in the system of the invention, since potentially cytotoxic amine structures of the nanocarrier like polyamine or polyethyleneamine structures are separated from the generally biocompatible polyglycerol core structure.

In another embodiment, said cleavage is performed by an enzyme. E.g., the nanocarrier comprised in the system of the invention may be designed in such a way that an esterase or a carbamate hydrolase may cleave the carbamate bond so that the polyglyceryl core is separated from the surrounding amine-containing surface or shell.

Hence, the present invention provides a system comprising at least one nanocarrier and at least one nucleic acid molecule, said nanocarrier being a compound having a structure according to formula (I),

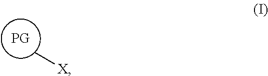

wherein PG denotes a linear or branched polyglycerol core, and X is covalently bound to a carbon atom of the polyglycerol core and is at each instance —NR¹R²,
wherein the polyglycerol core carries a plurality of groups of the type X,
R¹ is independently at each instance (i) H, (ii) linear or branched C₁-C₁₀-alkyl which may be substituted and/or interrupted by one or more oxygen, sulphur and/or nitrogen atoms, or by a group R³, or (iii) a group R³;
R² is independently at each instance (i) H, (ii) linear or branched C₁-C₁₀-alkyl which may be substituted and/or interrupted by one or more oxygen, sulphur and/or nitrogen atoms, or by a group R³, or (iii) R³;
R³ is —(CH₂CH₂NH)ₙ—H, wherein n is 1-100;
and said nucleic acid comprises a sequence denoted by any one of SEQ ID NO.123, SEQ ID NO.14, SEQ ID NO.117, SEQ ID NO.65, SEQ ID NO.70, SEQ ID NO.122, SEQ ID NO.32, SEQ ID NO.64, SEQ ID NO.63, SEQ ID NO.24, SEQ ID NO.108, SEQ ID NO.130, SEQ ID NO.131, SEQ ID NO.62, SEQ ID NO.15, SEQ ID NO.84 and SEQ ID NO.71, a complement thereof, or a sequence at least about 80% identical to any one of SEQ ID NO.123, SEQ ID NO.14, SEQ ID NO.117, SEQ ID NO.65, SEQ ID NO.70, SEQ ID NO.122, SEQ ID NO.32, SEQ ID NO.64, SEQ ID NO.63, SEQ ID NO.24, SEQ ID NO.108, SEQ ID NO.130, SEQ ID NO.131, SEQ ID NO.62, SEQ ID NO.15, SEQ ID NO.84 and SEQ ID NO.71, or a complement thereof.

In another further embodiment, the nucleic acid is to be carried by or bound to said nanocarrier in any one of a covalent, ionic or complexed manner. In one particular embodiment, said nucleic acid is RNA, particularly microRNA.

Again, all possible meanings for the microRNA as defined herein are also in the context of the claimed system to be understood as individually disclosed herein and to be optionally combined in any desired manner.

As referred to herein, microRNA or miRNA or miR may relate to the pri-miRNA or to the hairpin structure of the miR. A gene coding for a miR may be transcribed leading to production of a miR precursor known as the pri-miRNA. The pri-miRNA may be part of a polycistronic RNA comprising multiple pri-miRNAs. The pri-miRNA may form a hairpin with a stem and loop. The stem may comprise mismatched bases.

The hairpin structure of the pri-miRNA may be recognized by Drosha, which is an RNase III endonuclease. Drosha may recognize terminal loops in the pri-miRNA and cleave approximately two helical turns into the stem to produce a 30-200 nt precursor known as the pre-miRNA. Drosha may cleave the pri-miRNA with a staggered cut typical of RNase III endonucleases yielding a pre-miRNA stem loop with a 5′ phosphate and ˜2 nucleotide 3′ overhang. Approximately one helical turn of stem (˜10 nucleotides) extending beyond the Drosha cleavage site may be essential for efficient processing. The pre-miRNA may then be actively transported from the nucleus to the cytoplasm by Ran-GTP and the export receptor Ex-portin-5. The pre-miRNA may be part of a polycistronic RNA comprising multiple pre-miRNAs.

The pre-miRNA may be recognized by Dicer, which is also an RNase III endonuclease. Dicer may recognize the double-stranded stem of the pre-miRNA. Dicer may also recognize the 5′ phosphate and 3′ overhang at the base of the stem loop. Dicer may cleave off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The duplex-miRNA may be part of a polycistronic RNA comprising multiple miRNAs duplexes.

The miRNA and miRNA* may be derived from opposing arms of the pri-miRNA and pre-miRNA. MiRNA* sequences may be found in libraries of cloned miRNAs but typically at lower frequency than the miRNAs.

Although initially present as a double-stranded species with miRNA*, the miRNA may eventually become incorporated as a single-stranded RNA into a ribonucleoprotein complex known as the RNA-induced silencing complex (RISC). Various proteins can form the RISC, which can lead to variability in specifity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* may be removed and degraded. The strand of the miRNA:miRNA* duplex that is loaded into the RISC may be the strand whose 5′ end is less tightly paired. In cases where both ends of the miRNA:miRNA* have roughly equivalent 5′ pairing, both miRNA and miRNA* may have gene silencing activity.

The RISC may identify target nucleic acids based on high levels of complementarity between the miR and the mRNA, especially by nucleotides 2-8 of the miR. Only one case has been reported in animals where the interaction between the miR and its target was along the entire length of the miR. This was shown for mir-196 and Hox B8 and it was further shown that mir-196 mediates the cleavage of the Hox B8 mRNA (Yekta et al 2004, Science 304-594). Otherwise, such interactions are known only in plants (Bartel & Bartel 2003, Plant Physiol 132-709).

A number of studies have looked at the base-pairing requirement between miR and its mRNA target for achieving efficient inhibition of translation (reviewed by Bartel 2004, Cell 116-281). In mammalian cells, the first 8 nucleotides of the miR may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miR binding on whole genomes have suggested a specific role for bases 2-7 at the 5′ of the miR in target binding but the role of the first nucleotide, found usually to be “A” was also recognized (Lewis et at 2005 Cell 120-15). Similarly, nucleotides 1-7 or 2-8 were used to identify and validate targets by Krek et al (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miR binding sites in most genetically identified targets may indicate that the cooperative action of multiple RISCs provides the most efficient translational inhibition.

MiRNAs may direct the RISC to downregulate gene expression by either of two mechanisms: mRNA cleavage or translational repression. The miR may specify cleavage of the mRNA if the mRNA has a certain degree of complementarity to the miR. When a miR guides cleavage, the cut may be between the nucleotides pairing to residues 10 and 11 of the miR. Alternatively, the miR may repress translation if the miR does not have the requisite degree of complementarity to the miR. Translational repression may be more prevalent in animals since animals may have a lower degree of complementarity between the miR and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

Hence, the nucleic acid encapsulated by or complexed with the cationic carrier system may be RNA. Methods of RNA synthesis are well known in the art (Scaringe, S. A. Ph.D. Thesis, University of Colorado, 1996; Scaringe, S. A., et al., J. Am. Chem. Soc., 1998, 120, 11820-11821; Matteucci, M. D. and Caruthers, M. H. J. Am. Chem. Soc., 1981, 103, 3185-3191; Beaucage, S. L. and Caruthers, M. H. Tetrahedron Lett., 1981, 22, 1859-1862; Dahl, B. J., et al., Acta Chem. Scand. 1990, 44, 639-641; Reddy, M. P., et al., Tetrahedrom Lett., 1994, 25, 4311-4314; Wincott, F. et al., Nucleic Acids Res., 1995, 23, 2677-2684; Griffin, B. E., et al., Tetrahedron, 1967, 23, 2301-2313; Griffin, B. E., et al., Tetrahedron, 1967, 23, 2315-2331).

The nucleic acid encapsulated by or complexed with the cationic carrier system described herein may comprise a miR sequence as presented in Table 1, or a variant thereof.

TABLE 1
MiRNA Sequences

| | SEQ | |
|---|---|---|
| miR Name | ID NO. | miR Sequence |
| hsa-let-7f-5p | 1 | UGAGGUAGUAGAUUGUAUAGUU |
| hsa-miR-100-5p | 2 | AACCCGUAGAUCCGAACUUGUG |
| hsa-miR-103a-3p | 3 | AGCAGCAUUGUACAGGGCUAUGA |
| hsa-miR-107 | 4 | AGCAGCAUUGUACAGGGCUAUCA |
| hsa-miR-10b-5p | 5 | UACCCUGUAGAACCGAAUUUGUG |
| hsa-miR-1180-3p | 6 | UUUCCGGCUCGCGUGGGUGUGU |
| hsa-miR-1229-5p | 7 | GUGGGUAGGGUUUGGGGGAGAGC |
| | | G |
| hsa-miR-124-3p | 8 | UAAGGCACGCGGUGAAUGCC |
| hsa-miR-125b-2-3p | 9 | UCACAAGUCAGGCUCUUGGGAC |
| hsa-miR-1271-5p | 10 | CUUGGCACCUAGCAAGCACUCA |
| hsa-miR-1273f | 11 | GGAGAUGGAGGUUGCAGUG |
| hsa-miR-1285-3p | 12 | UCUGGGCAACAAAGUGAGACCU |
| hsa-miR-129-1-3p | 13 | AAGCCCUUACCCCAAAAAGUAU |
| hsa-miR-129-2-3p | 14 | AAGCCCUUACCCCAAAAAGCAU |
| hsa-miR-1290 | 15 | UGGAUUUUUGGAUCAGGGA |
| hsa-miR-130a-3p | 16 | CAGUGCAAUGUUAAAAGGGCAU |
| hsa-miR-130b-3p | 17 | CAGUGCAAUGAUGAAAGGGCAU |
| hsa-miR-132-3p | 18 | UAACAGUCUACAGCCAUGGUCG |
| hsa-miR-132-5p | 19 | ACCGUGGCUUUCGAUUGUUACU |
| hsa-miR-138-5p | 20 | AGCUGGUGUUGUGAAUCAGGCCG |
| hsa-miR-142-3p | 21 | UGUAGUGUUUCCUACUUUAUGGA |
| hsa-miR-148a-3p | 22 | UCAGUGCACUACAGAACUUUGU |
| hsa-miR-152-3p | 23 | UCAGUGCAUGACAGAACUUGG |
| hsa-miR-155-5p | 24 | UUAAUGCUAAUCGUGAUAGGGGU |
| hsa-miR-15b-5p | 25 | UAGCAGCACAUCAUGGUUUACA |
| hsa-miR-181a-2-3p | 26 | ACCACUGACCGUUGACUGUACC |
| hsa-miR-181c-3p | 27 | AACCAUCGACCGUUGAGUGGAC |
| hsa-miR-185-3p | 28 | AGGGGCUGGCUUUCCUCUGGUC |
| hsa-miR-185-5p | 29 | UGGAGAGAAAGGCAGUUCCUGA |
| hsa-miR-18a-5p | 30 | UAAGGUGCAUCUAGUGCAGAUAG |
| hsa-miR-18b-5p | 31 | UAAGGUGCAUCUAGUGCAGUUAG |
| hsa-miR-195-5p | 32 | UAGCAGCACAGAAAUAUUGGC |
| hsa-miR-199a-3p | 33 | ACAGUAGUCUGCACAUUGGUUA |
| hsa-miR-199a-5p | 34 | CCCAGUGUUCAGACUACCUGUUC |
| hsa-miR-19b-3p | 35 | UGUGCAAAUCCAUGCAAAACUGA |
| hsa-miR-204-5p | 36 | UUCCCUUUGUCAUCCUAUGCCU |
| hsa-miR-210-3p | 37 | CUGUGCGUGUGACAGCGGCUGA |
| hsa-miR-212-3p | 38 | UAACAGUCUCCAGUCACGGCC |
| hsa-miR-214-3p | 39 | ACAGCAGGCACAGACAGGCAGU |
| hsa-miR-221-3p | 40 | AGCUACAUUGUCUGCUGGGUUUC |
| hsa-miR-222-3p | 41 | AGCUACAUCUGGCUACUGGGU |
| hsa-miR-224-5p | 42 | CAAGUCACUAGUGGUUCCGUU |
| hsa-miR-26b-5p | 43 | UUCAAGUAAUUCAGGAUAGGU |
| hsa-miR-29a-3p | 44 | UAGCACCAUCUGAAAUCGGUUA |
| hsa-miR-301a-3p | 45 | CAGUGCAAUAGUAUUGUCAAAGC |
| hsa-miR-30a-3p | 46 | CUUUCAGUCGGAUGUUUGCAGC |
| hsa-miR-30a-5p | 47 | UGUAAACAUCCUCGACUGGAAG |
| hsa-miR-30b-5p | 48 | UGUAAACAUCCUACACUCAGCU |
| hsa-miR-30c-2-3p | 49 | CUGGGAGAAGGCUGUUUACUCU |
| hsa-miR-30c-5p | 50 | UGUAAACAUCCUACACUCUCAGC |
| hsa-miR-30d-5p | 51 | UGUAAACAUCCCCGACUGGAAG |
| hsa-miR-30e-3p | 52 | CUUUCAGUCGGAUGUUUACAGC |
| hsa-miR-30e-5p | 53 | UGUAAACAUCCUUGACUGGAAG |
| hsa-miR-3185 | 54 | AGAAGAAGGCGGUCGGUCUGCGG |
| hsa-miR-3200-3p | 55 | CACCUUGCGCUACUCAGGUCUG |
| hsa-miR-323a-3p | 56 | CACAUUACACGGUCGACCUCU |
| hsa-miR-324-3p | 57 | ACUGCCCCAGGUGCUGCUGG |
| hsa-miR-329-3p | 58 | AACACACCUGGUUAACCUCUUU |
| hsa-miR-330-3p | 59 | GCAAAGCACACGGCCUGCAGAGA |
| hsa-miR-339-5p | 60 | UCCCUGUCCUCCAGGAGCUCACG |
| hsa-miR-345-5p | 61 | GCUGACUCCUAGUCCAGGGCUC |
| hsa-miR-34a-3p | 62 | CAAUCAGCAAGUAUACUGCCCU |
| hsa-miR-34a-5p | 63 | UGGCAGUGUCUUAGCUGGUUGU |
| hsa-miR-34b-5p | 64 | UAGGCAGUGUCAUUAGCUGAUUG |
| hsa-miR-34c-5p | 65 | AGGCAGUGUAGUUAGCUGAUUGC |
| hsa-miR-3607-3p | 66 | ACUGUAAACGCUUUCUGAUG |
| hsa-miR-361-5p | 67 | UUAUCAGAAUCUCCAGGGGUAC |
| hsa-miR-3653 | 68 | CUAAGAAGUUGACUGAAG |
| hsa-miR-374a-5p | 69 | UUAUAAUACAACCUGAUAAGUG |
| hsa-miR-374b-5p | 70 | AUAUAAUACAACCUGCUAAGUG |
| hsa-miR-374c-5p | 71 | AUAAUACAACCUGCUAAGUGCU |
| hsa-miR-378a-3p | 72 | ACUGGACUUGGAGUCAGAAGGC |
| hsa-miR-378c | 73 | ACUGGACUUGGAGUCAGAAGAGU |
| | | GG |
| hsa-miR-378d | 74 | ACUGGACUUGGAGUCAGAAA |
| hsa-miR-378g | 75 | ACUGGGCUUGGAGUCAGAAG |
| hsa-miR-378i | 76 | ACUGGACUAGGAGUCAGAAGG |
| hsa-miR-3907 | 77 | AGGUGCUCCAGGCUGGCUCACA |
| hsa-miR-421 | 78 | AUCAACAGACAUUAAUUGGGCGC |
| hsa-miR-422a | 79 | ACUGGACUUGGAGUCAGAAGGC |
| hsa-miR-423-5p | 80 | UGAGGGGCAGAGAGCGAGACUUU |
| hsa-miR-424-5p | 81 | CAGCAGCAAUUCAUGUUUUGAA |
| hsa-miR-425-5p | 82 | AAUGACACGAUCACUCCCGUUGA |
| hsa-miR-4306 | 83 | UGGAGAGAAAGGCAGUA |
| hsa-miR-4324 | 84 | CCCUGAGACCCUAACCUUAA |
| hsa-miR-4454 | 85 | GGAUCCGAGUCACGGCACCA |
| hsa-miR-451a | 86 | AAACCGUUACCAUUACUGAGUU |
| hsa-miR-4521 | 87 | GCUAAGGAAGUCCUGUGCUCAG |
| hsa-miR-454-3p | 88 | UAGUGCAAUAUUGCUUAUAGGGU |
| hsa-miR-455-3p | 89 | GCAGUCCAUGGGCAUAUACAC |
| hsa-miR-4640-5p | 90 | UGGGCCAGGGAGCAGCUGGUGGG |
| hsa-miR-4656 | 91 | UGGGCUGAGGGCAGGAGGCCUGU |
| hsa-miR-4664-5p | 92 | UGGGGUGCCCACUCCGCAAGUU |
| hsa-miR-4690-5p | 93 | GAGCAGGCGAGGCUGGGCUGAA |
| hsa-miR-4695-5p | 94 | CAGGAGGCAGUGGGCGAGCAGG |
| hsa-miR-4732-5p | 95 | UGUAGAGCAGGGAGCAGGAAGCU |
| hsa-miR-4800-3p | 96 | CAUCCGUCCGUCUGUCCAC |
| hsa-miR-487b-3p | 97 | AAUCGUACAGGGUCAUCCACUU |
| hsa-miR-497-5p | 98 | CAGCAGCACACUGUGGUUUGU |
| hsa-miR-5001-5p | 99 | AGGGCUGGACUCAGCGGCGGAGC |
| | | U |
| hsa-miR-5010-5p | 100 | AGGGGGAUGGCAGAGCAAAAUU |
| hsa-miR-503-5p | 101 | UAGCAGCGGGAACAGUUCUGCAG |
| hsa-miR-504-5p | 102 | AGACCCUGGUCUGCACUCUAUC |
| hsa-miR-505-5p | 103 | GGGAGCCAGGAAGUAUUGAUGU |
| hsa-miR-5100 | 104 | UUCAGAUCCCAGCGGUGCCUCU |
| hsa-miR-532-5p | 105 | CAUGCCUUGAGUGUAGGACCGU |
| hsa-miR-542-5p | 106 | UCGGGGAUCAUCAUGUCACGAGA |
| hsa-miR-5684 | 107 | AACUCUAGCCUGAGCAACAG |
| hsa-miR-584-5p | 108 | UUAUGGUUUGCCUGGGACUGAG |
| hsa-miR-625-5p | 109 | AGGGGGAAAGUUCUAUAGUCC |
| hsa-miR-629-5p | 110 | UGGGUUUACGUUGGGAGAACU |
| hsa-miR-6511a-5p | 111 | CAGGCAGAAGUGGGGCUGACAGG |
| hsa-miR-652-3p | 112 | AAUGGCGCCACUAGGGUUGUG |
| hsa-miR-664b-3p | 113 | UUCAUUUGCCUCCCAGCCUACA |
| hsa-miR-671-5p | 114 | AGGAAGCCCUGGAGGGGCUGGAG |
| hsa-miR-6723-5p | 115 | AUAGUCCGAGUAACGUCGGGGC |
| hsa-miR-708-3p | 116 | CAACUAGACUGUGAGCUUCUAG |
| hsa-miR-708-5p | 117 | AAGGAGCUUACAAUCUAGCUGGG |
| hsa-miR-744-5p | 118 | UGCGGGGCUAGGGCUAACAGCA |
| hsa-miR-769-5p | 119 | UGAGACCUCUGGGUUCUGAGCU |
| hsa-miR-9-3p | 120 | AUAAAGCUAGAUAACCGAAAGU |
| hsa-miR-9-5p | 121 | UCUUUGGUUAUCUAGCUGUAUGA |
| hsa-miR-99a-3p | 122 | CAAGCUCGCUUCUAUGGGUCUG |
| hsa-miR-99a-5p | 123 | AACCCGUAGAUCCGAUCUUGUG |
| hsa-miR-99b-3p | 124 | CAAGCUCGUGUCUGUGGGUCCG |
| MID-00206 | 125 | AUAUGCCAUGUUGGUGUGCUGC |
| MID-00612 | 126 | UCGAGGCUAGAGUCACGCUUGG |
| MID-00689 | 127 | UGGACUUGGAGUCAGGAGGCCU |
| MID-00713 | 128 | UGGUGUGCUAGAGUACUCGAAG |
| MID-00745 | 129 | UGUGCCAUGUUGGUGUGCUGCA |
| MID-01140 | 130 | CGCGGGUGCUUACUGACCCUU |
| MID-01141 | 131 | CGGGUCGGAGUUAGCUCAAGCGG |
| MID-14865 | 132 | AUUGAUCAUCGACACUUCGAACG |
| | | CAAU |
| MID-15867 | 133 | ACAUGAAAAGGGGAGAGGGCA |
| MID-16318 | 134 | AGCUGGUUUGAUGGGGAGCCAU |
| MID-16752 | 135 | AUCCCGGACGAGCCCCCAUUU |
| MID-17144 | 136 | CACUGAUUAUCGAGGCGAUUCU |
| MID-18307 | 137 | GAAAGCUGAGCGUGAACGUG |
| MID-18336 | 138 | GAACCCUACUCCUGGUACCA |
| MID-18468 | 139 | GAC GUGAGGGGGUGCUACAUAC |
| MID-19149 | 140 | GCUUGUUGAAAGGCGCGGGCUC |
| MID-19433 | 141 | GGCUGGUCCGAAGGUAGUGAGUU |
| MID-19434 | 142 | GGCUGGUCCGAGUGCAGUGGUGU |
| | | UU |
| MID-19898 | 143 | GUUCCUGUUGGCCGAGUGGAGAC |
| MID-23291 | 144 | UGUGGGUUCGAGUUCCAU |
| MID-24702 | 145 | CCCCCACAACCGCGCUUGACUAG |
| | | CU |
| NC (guide) | 146 | UGGACUCUGAGAAAGGAGUAUG |
| NC (passenger) | 147 | UACUCCUUAUCAGACUCCAUA |

miR name is the miRBase registry name (release 20), except for the miR names represented by MID-numeral.
miRs represented by MID-numeral were predicted and/or cloned at Rosetta Genomics.

The nucleic acid encapsulated by or complexed with the cationic carrier system described herein may alternatively comprise a miR hairpin sequence as presented in Table 2, or a variant thereof.

TABLE 2
Hairpin sequences

| miR Name | SEQ ID NO. | Hairpin Sequence |
|---|---|---|
| hsa-let-7f-1 | 152 | UCAGAGUGAGGUAGUAGAUUGUAUAGUUGUGGGGUAGUGA |
| | | UUUUACCCUGUUCAGGAGAUAACUAUACAAUCUAUUGCCU |
| | | UCCCUGA |
| hsa-let-7f-2 | 153 | UGUGGGAUGAGGUAGUAGAUUGUAUAGUUUUAGGGUCAUA |
| | | CCCCAUCUUGGAGAUAACUAUACAGUCUACUGUCUUUCCC |
| | | ACG |
| hsa-miR-100 | 154 | CCUGUUGCCACAAACCCGUAGAUCCGAACUUGUGGUAUUA |
| | | GUCCGCACAAGCUUGUAUCUAUAGGUAUGUGUCUGUUAGG |
| hsa-miR-103a-2 | 155 | UUGUGCUUUCAGCUUCUUUACAGUGCUGCCUUGUAGCAUU |
| | | CAGGUCAAGCAGCAUUGUACAGGGCUAUGAAAGAACCA |
| hsa-miR-103a-1 | 156 | UACUGCCCUCGGCUUCUUUACAGUGCUGCCUUGUUGCAUA |
| | | UGGAUCAAGCAGCAUUGUACAGGGCUAUGAAGGCAUUG |
| hsa-miR-107 | 157 | CUCUCUGCUUUCAGCUUCUUUACAGUGUUGCCUUGUGGCA |
| | | UGGAGUUCAAGCAGCAUUGUACAGGGCUAUCAAAGCACAG |
| | | A |
| hsa-miR-10b | 158 | CCAGAGGUUGUAACGUUGUCUAUAUAUACCCUGUAGAACC |
| | | GAAUUUGUGUGGUAUCCGUAUAGUCACAGAUUCGAUUCUA |
| | | GGGGAAUAUAUGGUCGAUGCAAAAACUUCA |
| hsa-miR-1180 | 159 | GCUGCUGGACCCACCCGGCCGGGAAUAGUGCUCCUGGUUG |
| | | UUUCCGGCUCGCGUGGGUGUGUCGGCGGC |
| hsa-miR-1229 | 160 | GUGGGUAGGGUUUGGGGGAGAGCGUGGGCUGGGGUUCAGG |
| | | GACACCCUCUCACCACUGCCCUCCCACAG |
| hsa-miR-124-1 | 161 | AGGCCUCUCUCUCCGUGUUCACAGCGGACCUUGAUUUAAA |
| | | UGUCCAUACAAUUAAGGCACGCGGUGAAUGCCAAGAAUGG |
| | | GGCUG |
| hsa-miR-124-3 | 162 | UGAGGGCCCCUCUGCGUGUUCACAGCGGACCUUGAUUUAA |
| | | UGUCUAUACAAUUAAGGCACGCGGUGAAUGCCAAGAGAGG |
| | | CGCCUCC |
| hsa-miR-124-2 | 163 | AUCAAGAUUAGAGGCUCUGCUCUCCGUGUUCACAGCGGAC |
| | | CUUGAUUUAAUGUCAUACAAUUAAGGCACGCGGUGAAUGC |
| | | CAAGAGCGGAGCCUACGGCUGCACUUGAA |
| hsa-miR-125b-2 | 164 | ACCAGACUUUUCCUAGUCCCUGAGACCCUAACUUGUGAGG |
| | | UAUUUUAGUAACAUCACAAGUCAGGCUCUUGGGACCUAGG |
| | | CGGAGGGGA |
| hsa-miR-1271 | 165 | CACCCAGAUCAGUGCUUGGCACCUAGCAAGCACUCAGUAA |
| | | AUAUUUGUUGAGUGCCUGCUAUGUGCCAGGCAUUGUGCUG |
| | | AGGGCU |
| hsa-miR-1273f | 166 | AGGUGGGAGGAUUGCUUGAGCCUGGGAGAUGGAGGUUGCA |
| | | GUGAGCUGAGAUCACGCAACUGCACCCCCAGCCUGGGCCA |
| | | UAGAGUCAGUCCUUGUCUC |
| hsa-miR-1285-2 | 167 | UUUGGGAGGCCGAGGCUGGUGCAUCACUUGAGCCCAGCAA |
| | | UUUGAGACCAAUCUGGGCAACAAAGUGAGACCUCCGUCUC |
| | | UACAAAGA |
| hsa-miR-1285-1 | 168 | UGUAGAGAUAGGAUCUCACUUUGUUGCCCAGGCUGGUCUC |
| | | AAACUCCUGGUCUGGGCAACAAAGUGAGACCUUAUCUCUA |
| | | CAAG |
| hsa-miR-129-1 | 169 | GGAUCUUUUUGCGGUCUGGGCUUGCUGUUCCUCUCAACAG |
| | | UAGUCAGGAAGCCCUUACCCCAAAAAGUAUCU |
| hsa-miR-129-2 | 170 | UGCCCUUCGCGAAUCUUUUUGCGGUCUGGGCUUGCUGUAC |
| | | AUAACUCAAUAGCCGGAAGCCCUUACCCCAAAAAGCAUUU |
| | | GCGGAGGGCG |
| hsa-miR-1290 | 171 | GAGCGUCACGUUGACACUCAAAAAGUUUCAGAUUUUGGAA |
| | | CAUUUCGGAUUUUGGAUUUUUGGAUCAGGGAUGCUCAA |
| hsa-miR-130a | 172 | UGCUGCUGGCCAGAGCUCUUUUCACAUUGUGCUACUGUCU |
| | | GCACCUGUCACUAGCAGUGCAAUGUUAAAAGGGCAUUGGC |
| | | CGUGUAGUG |
| hsa-miR-130b | 173 | GGCCUGCCCGACACUCUUUCCCUGUUGCACUACUAUAGGC |
| | | CGCUGGGAAGCAGUGCAAUGAUGAAAGGGCAUCGGUCAGG |
| | | UC |
| hsa-miR-132-3p | 174 | CCGCCCCCGCGUCUCCAGGGCAACCGUGGCUUUCGAUUGU |
| | | UACUGUGGGAACUGGAGGUAACAGUCUACAGCCAUGGUCG |
| | | CCCCGCAGCACGCCCACGCGC |
| hsa-miR-138-2 | 175 | CGUUGCUGCAGCUGGUGUUGUGAAUCAGGCCGACGAGCAG |
| | | CGCAUCCUCUUACCCGGCUAUUUCACGACACCAGGGUUGC |
| | | AUCA |
| hsa-miR-138-1 | 176 | CCCUGGCAUGGUGUGGUGGGGCAGCUGGUGUUGUGAAUCA |
| | | GGCCGUUGCCAAUCAGAGAACGGCUACUUCACAACACCAG |
| | | GGCCACACCACACUACAGG |
| hsa-miR-142 | 177 | GACAGUGCAGUCACCCAUAAAGUAGAAAGCACUACUAACA |
| | | GCACUGGAGGGUGUAGUGUUUCCUACUUUAUGGAUGAGUG |
| | | UACUGUG |
| hsa-miR-148a | 178 | GAGGCAAAGUUCUGAGACACUCCGACUCUGAGUAUGAUAG |
| | | AAGUCAGUGCACUACAGAACUUUGUCUC |
| hsa-miR-152 | 179 | UGUCCCCCCCGGCCCAGGUUCUGUGAUACACUCCGACUCG |
| | | GGCUCUGGAGCAGUCAGUGCAUGACAGAACUUGGGCCCGG |
| | | AAGGACC |
| hsa-miR-155 | 180 | CUGUUAAUGCUAAUCGUGAUAGGGGUUUUUGCCUCCAACU |
| | | GACUCCUACAUAUUAGCAUUAACAG |
| hsa-miR-15b | 181 | UUGAGGCCUUAAAGUACUGUAGCAGCACAUCAUGGUUUAC |
| | | AUGCUACAGUCAAGAUGCGAAUCAUUAUUUGCUGCUCUAG |
| | | AAAUUUAAGGAAAUUCAU |
| hsa-miR-181a-2 | 182 | AGAAGGGCUAUCAGGCCAGCCUUCAGAGGACUCCAAGGAA |
| | | CAUUCAACGCUGUCGGUGAGUUUGGGAUUUGAAAAAACCA |
| | | CUGACCGUUGACUGUACCUUGGGGUCCUUA |
| hsa-miR-181c | 183 | CGGAAAAUUUGCCAAGGGUUUGGGGGAACAUUCAACCUGU |
| | | CGGUGAGUUUGGGCAGCUCAGGCAAACCAUCGACCGUUGA |
| | | GUGGACCCUGAGGCCUGGAAUUGCCAUCCU |
| hsa-miR-185 | 184 | AGGGGGCGAGGGAUUGGAGAGAAAGGCAGUUCCUGAUGGU |
| | | CCCCUCCCCAGGGGCUGGCUUUCCUCUGGUCCUUCCCUCC |
| | | CA |
| hsa-miR-18a | 185 | UGUUCUAAGGUGCAUCUAGUGCAGAUAGUGAAGUAGAUUA |
| | | GCAUCUACUGCCCUAAGUGCUCCUUCUGGCA |
| hsa-miR-18b | 186 | UGUGUUAAGGUGCAUCUAGUGCAGUUAGUGAAGCAGCUUA |
| | | GAAUCUACUGCCCUAAAUGCCCCUUCUGGCA |
| hsa-miR-195 | 187 | AGCUUCCCUGGCUCUAGCAGCACAGAAAUAUUGGCACAGG |
| | | GAAGCGAGUCUGCCAAUAUUGGCUGUGCUGCUCCAGGCAG |
| | | GGUGGUG |
| hsa-miR-199a-2 | 188 | AGGAAGCUUCUGGAGAUCCUGCUCCGUCGCCCCAGUGUUC |
| | | AGACUACCUGUUCAGGACAAUGCCGUUGUACAGUAGUCUG |
| | | CACAUUGGUUAGACUGGGCAAGGGAGAGCA |
| hsa-miR-199a-1 | 189 | GCCAACCCAGUGUUCAGACUACCUGUUCAGGAGGCUCUCA |
| | | AUGUGUACAGUAGUCUGCACAUUGGUUAGGC |
| hsa-miR-19b-1 | 190 | CACUGUUCUAUGGUUAGUUUUGCAGGUUUGCAUCCAGCUG |
| | | UGUGAUAUUCUGCUGUGCAAAUCCAUGCAAAACUGACUGU |
| | | GGUAGUG |
| hsa-miR-19b-2 | 191 | ACAUUGCUACUUACAAUUAGUUUUGCAGGUUUGCAUUUCA |
| | | GCGUAUAUAUGUAUAUGUGGCUGUGCAAAUCCAUGCAAAA |
| | | CUGAUUGUGAUAAUGU |
| hsa-miR-204 | 192 | GGCUACAGUCUUUCUUCAUGUGACUCGUGGACUUCCCUUU |
| | | GUCAUCCUAUGCCUGAGAAUAUAUGAAGGAGGCUGGGAAG |
| | | GCAAAGGGACGUUCAAUUGUCAUCACUGGC |
| hsa-miR-210 | 193 | ACCCGGCAGUGCCUCCAGGCGCAGGGCAGCCCCUGCCCAC |
| | | CGCACACUGCGCUGCCCCAGACCCACUGUGCGUGUGACAG |
| | | CGGCUGAUCUGUGCCUGGGCAGCGCGACCC |
| hsa-miR-212 | 194 | CGGGGCACCCCGCCCGGACAGCGCGCCGGCACCUUGGCUC |
| | | UAGACUGCUUACUGCCCGGGCCGCCCUCAGUAACAGUCUC |
| | | CAGUCACGGCCACCGACGCCUGGCCCCGCC |
| hsa-miR-214 | 195 | GGCCUGGCUGGACAGAGUUGUCAUGUGUCUGCCUGUCUAC |
| | | ACUUGCUGUGCAGAACAUCCGCUCACCUGUACAGCAGGCA |
| | | CAGACAGGCAGUCACAUGACAACCCAGCCU |
| hsa-miR-221 | 196 | UGAACAUCCAGGUCUGGGGCAUGAACCUGGCAUACAAUGU |
| | | AGAUUUCUGUGUUCGUUAGGCAACAGCUACAUUGUCUGCU |
| | | GGGUUUCAGGCUACCUGGAAACAUGUUCUC |
| hsa-miR-222 | 197 | GCUGCUGGAAGGUGUAGGUACCCUCAAUGGCUCAGUAGCC |
| | | AGUGUAGAUCCUGUCUUUCGUAAUCAGCAGCUACAUCUGG |
| | | CUACUGGGUCUCUGAUGGCAUCUUCUAGCU |
| hsa-miR-224 | 198 | GGGCUUUCAAGUCACUAGUGGUUCCGUUUAGUAGAUGAUU |
| | | GUGCAUUGUUUCAAAAUGGUGCCCUAGUGACUACAAAGCC |
| | | C |
| hsa-miR-26b | 199 | CCGGGACCCAGUUCAAGUAAUUCAGGAUAGGUUGUGUGCU |
| | | GUCCAGCCUGUUCUCCAUUACUUGGCUCGGGGACCGG |
| hsa-miR-29a | 200 | AUGACUGAUUUCUUUUGGUGUUCAGAGUCAAUAUAAUUUU |
| | | CUAGCACCAUCUGAAAUCGGUUAU |
| hsa-miR-301a | 201 | ACUGCUAACGAAUGCUCUGACUUUAUUGCACUACUGUACU |
| | | UUACAGCUAGCAGUGCAAUAGUAUUGUCAAAGCAUCUGAA |
| | | AGCAGG |
| hsa-miR-30a | 202 | GCGACUGUAAACAUCCUCGACUGGAAGCUGUGAAGCCACA |
| | | GAUGGGCUUUCAGUCGGAUGUUUGCAGCUGC |
| hsa-miR-30b | 203 | ACCAAGUUUCAGUUCAUGUAAACAUCCUACACUCAGCUGU |
| | | AAUACAUGGAUUGGCUGGGAGGUGGAUGUUUACUUCAGCU |
| | | GACUUGGA |
| | | AGAUACUGUAAACAUCCUACACUCUCAGCUGUGGAAAGUA |
| hsa-miR-30c-2 | 204 | AGAAAGCUGGGAGAAGGCUGUUUACUCUUUCU |
| hsa-miR-30c-1 | 205 | ACCAUGCUGUAGUGUGUGUAAACAUCCUACACUCUCAGCU |
| | | GUGAGCUCAAGGUGGCUGGGAGAGGGUUGUUUACUCCUUC |
| | | UGCCAUGGA |
| hsa-miR-30d | 206 | GUUGUUGUAAACAUCCCCGACUGGAAGCUGUAAGACACAG |
| | | CUAAGCUUUCAGUCAGAUGUUUGCUGCUAC |
| hsa-miR-30e | 207 | GGGCAGUCUUUGCUACUGUAAACAUCCUUGACUGGAAGCU |
| | | GUAAGGUGUUCAGAGGAGCUUUCAGUCGGAUGUUUACAGC |
| | | GGCAGGCUGCCA |
| hsa-miR-3185 | 208 | GAAUGGAAGAAGAAGGCGGUCGGUCUGCGGGAGCCAGGCC |
| | | GCAGAGCCAUCCGCCUUCUGUCCAUGUC |
| hsa-miR-3200 | 209 | GGUGGUCGAGGGAAUCUGAGAAGGCGCACAAGGUUUGUGU |
| | | CCAAUACAGUCCACACCUUGCGCUACUCAGGUCUGCUCGU |
| | | GCCCU |
| hsa-miR-323a | 210 | UUGGUACUUGGAGAGAGGUGGUCCGUGGCGCGUUCGCUUU |
| | | AUUUAUGGCGCACAUUACACGGUCGACCUCUUUGCAGUAU |
| | | CUAAUC |
| hsa-miR-324 | 211 | CUGACUAUGCCUCCCCGCAUCCCCUAGGGCAUUGGUGUAA |
| | | AGCUGGAGACCCACUGCCCCAGGUGCUGCUGGGGGUUGUA |
| | | GUC |
| hsa-miR-329-1 | 212 | UGAGGACGAAACACACCUGGUUAACCUCUUUUCCAGUAUC |
| | | GUGGUACCUGAAGAGAGGUUUUCUGGGUUUCUGUUUCUUU |
| hsa-miR-329-2 | 213 | AUUGAGGACGAAACACACCUGGUUAACCUCUUUUCCAGUA |
| | | UCAA |
| hsa-miR-330 | 214 | CUUUGGCGAUCACUGCCUCUCUGGGCCUGUGUCUUAGGCU |
| | | CUGCAAGAUCAACCGAGCAAAGCACACGGCCUGCAGAGAG |
| | | GCAGCGCUCUGCCC |
| hsa-miR-339 | 215 | CGGGGCGGCCGCUCUCCCUGUCCUCCAGGAGCUCACGUGU |
| | | GCCUGCCUGUGAGCGCCUCGACGACAGAGCCGGCGCCUGC |
| | | CCCAGUGUCUGCGC |
| hsa-miR-345 | 216 | ACCCAAACCCUAGGUCUGCUGACUCCUAGUCCAGGGCUCG |
| | | UGAUGGCUGGUGGGCCCUGAACGAGGGGUCUGGAGGCCUG |
| | | GGUUUGAAUAUCGACAGC |
| hsa-miR-34a | 217 | GGCCAGCUGUGAGUGUUUCUUUGGCAGUGUCUUAGCUGGU |
| | | UGUUGUGAGCAAUAGUAAGGAAGCAAUCAGCAAGUAUACU |
| | | GCCCUAGAAGUGCUGCACGUUGUGGGGCCC |
| hsa-miR-34b | 218 | GUGCUCGGUUUGUAGGCAGUGUCAUUAGCUGAUUGUACUG |
| | | UGGUGGUUACAAUCACUAACUCCACUGCCAUCAAAACAAG |
| | | GCAC |
| hsa-miR-34c | 219 | AGUCUAGUUACUAGGCAGUGUAGUUAGCUGAUUGCUAAUA |
| | | GUACCAAUCACUAACCACACGGCCAGGUAAAAAGAUU |
| hsa-miR-3607 | 220 | AAGGUUGCGGUGCAUGUGAUGAAGCAAAUCAGUAUGAAUG |
| | | AAUUCAUGAUACUGUAAACGCUUUCUGAUGUACUACUCA |
| hsa-miR-361 | 221 | GGAGCUUAUCAGAAUCUCCAGGGGUACUUUAUAAUUUCAA |
| | | AAAGUCCCCCAGGUGUGAUUCUGAUUUGCUUC |
| hsa-miR-3653 | 222 | UCCCUGGGGACCCCUGGCAGCCCCUCCUGAUGAUUCUUCU |
| | | UCCUGAGCACGCUCAUGAUGAGCAAACUGAGCCUCUAAGA |
| | | AGUUGACUGAAGGGGCUGCUUCCCCAAGGA |
| hsa-miR-374a | 223 | UACAUCGGCCAUUAUAAUACAACCUGAUAAGUGUUAUAGC |
| | | ACUUAUCAGAUUGUAUUGUAAUUGUCUGUGUA |
| | | ACUCGGAUGGAUAUAAUACAACCUGCUAAGUGUCCUAGCA |
| hsa-miR-374b | 224 | CUUAGCAGGUUGUAUUAUCAUUGUCCGUGUCU |
| hsa-miR-374c | 225 | ACACGGACAAUGAUAAUACAACCUGCUAAGUGCUAGGACA |
| | | CUUAGCAGGUUGUAUUAUAUCCAUCCGAGU |
| hsa-miR-378a | 226 | AGGGCUCCUGACUCCAGGUCCUGUGUGUUACCUAGAAAUA |
| | | GCACUGGACUUGGAGUCAGAAGGCCU |
| hsa-miR-378c | 227 | GGAGGCCAUCACUGGACUUGGAGUCAGAAGAGUGGAGUCG |
| | | GGUCAGACUUCAACUCUGACUUUGAAGGUGGUGAGUGCCU |
| | | C |
| hsa-miR-378d-2 | 228 | GAAUGGUUACAAGGAGAGAACACUGGACUUGGAGUCAGAA |
| | | AACUUUCAUCCAAGUCAUUCCCUGCUCUAAGUCCCAUUUC |
| | | UGUUCCAUGAGAUUGUUU |
| hsa-miR-378d-1 | 229 | ACUGUUUCUGUCCUUGUUCUUGUUGUUAUUACUGGACUUG |
| | | GAGUCAGAAACAGG |
| hsa-miR-378g | 230 | CACUGGGCUUGGAGUCAGAAGACCUGGCUCCAGCCCAGCU |
| | | C |
| hsa-miR-378i | 231 | GGGAGCACUGGACUAGGAGUCAGAAGGUGGAGUUCUGGGU |
| | | GCUGUUUUCCCACUCUUGGGCCCUGGGCAUGUUCUG |
| hsa-miR-3907 | 232 | GGGUUGGAAAGCUGUAGGUGUGGAGGGGCAUGGAUACGGG |
| | | GGCCAUGAGGGUGGGGUCCAGGCUGGACCAGGCCUGCCCU |
| | | GAGUCCCCCAGCAGGUGCUCCAGGCUGGCUCACACCCUCU |
| | | GCCUCUCUCUCUUCCUUCCUGGCCCCAACCC |
| hsa-miR-421 | 233 | GCACAUUGUAGGCCUCAUUAAAUGUUUGUUGAAUGAAAAA |
| | | AUGAAUCAUCAACAGACAUUAAUUGGGCGCCUGCUCUGUG |
| | | AUCUC |
| hsa-miR-422a | 234 | GAGAGAAGCACUGGACUUAGGGUCAGAAGGCCUGAGUCUC |
| | | UCUGCUGCAGAUGGGCUCUCUGUCCCUGAGCCAAGCUUUG |
| | | UCCUCCCUGG |
| hsa-miR-423 | 235 | AUAAAGGAAGUUAGGCUGAGGGGCAGAGAGCGAGACUUUU |
| | | CUAUUUUCCAAAAGCUCGGUCUGAGGCCCCUCAGUCUUGC |
| | | UUCCUAACCCGCGC |
| hsa-miR-424 | 236 | CGAGGGGAUACAGCAGCAAUUCAUGUUUUGAAGUGUUCUA |
| | | AAUGGUUCAAAACGUGAGGCGCUGCUAUACCCCCUCGUGG |
| | | GGAAGGUAGAAGGUGGGG |
| hsa-miR-425 | 237 | GAAAGCGCUUUGGAAUGACACGAUCACUCCCGUUGAGUGG |
| | | GCACCCGAGAAGCCAUCGGGAAUGUCGUGUCCGCCCAGUG |
| | | CUCUUUC |
| hsa-miR-4306 | 238 | AAGCUGCUUAGUGUCCUUAGAGUCUCCAGAGGCAUCCCUA |
| | | ACCCAGAAUCUUUUGACUGUCCUCUGGAGAGAAAGGCAGU |
| | | AGGUCUGUACC |
| hsa-miR-4324 | 239 | CGGCCCCUUUGUUAAGGGUCUCAGCUCCAGGGAACUUUAA |
| | | AACCCUGAGACCCUAACCUUAAAGGUGCUGCA |
| hsa-miR-4454 | 240 | CCGGAUCCGAGUCACGGCACCAAAUUUCAUGCGUGUCCGU |
| | | GUGAAGAGACCACCA |
| hsa-miR-451a | 241 | CUUGGGAAUGGCAAGGAAACCGUUACCAUUACUGAGUUUA |
| | | GUAAUGGUAAUGGUUCUCUUGCUAUACCCAGA |
| hsa-miR-4521 | 242 | UCGGCUAAGGAAGUCCUGUGCUCAGUUUUGUAGCAUCAAA |
| | | ACUAGGAUUUCUCUUGUUAC |
| hsa-miR-454 | 243 | UCUGUUUAUCACCAGAUCCUAGAACCCUAUCAAUAUUGUC |
| | | UCUGCUGUGUAAAUAGUUCUGAGUAGUGCAAUAUUGCUUA |
| | | UAGGGUUUUGGUGUUUGGAAAGAACAAUGGGCAGG |
| hsa-miR-455 | 244 | UCCCUGGCGUGAGGGUAUGUGCCUUUGGACUACAUCGUGG |
| | | AAGCCAGCACCAUGCAGUCCAUGGGCAUAUACACUUGCCU |
| | | CAAGGCCUAUGUCAUC |
| hsa-miR-4640 | 245 | CUGUGGGCUGGGCCAGGGAGCAGCUGGUGGGUGGGAAGUA |
| | | AGAUCUGACCUGGACUCCAUCCCACCCACCCCCUGUUUCC |
| | | UGGCCCACAG |
| hsa-miR-4656 | 246 | AGGCUGGCGUGGGCUGAGGGCAGGAGGCCUGUGGCCGGUC |
| | | CCAGGCCUCCUGCUUCCUGGGCUCAGGCUCGGUUU |
| hsa-miR-4664 | 247 | GUUGGGGGCUGGGGUGCCCACUCCGCAAGUUAUCACUGAG |
| | | CGACUUCCGGUCUGUGAGCCCCGUCCUCCGC |
| hsa-miR-4690 | 248 | GAGCAGGCGAGGCUGGGCUGAACCCGUGGGUGAGGAGUGC |
| | | AGCCCAGCUGAGGCCUCUGC |
| hsa-miR-4695 | 249 | CCUGCAGGAGGCAGUGGGCGAGCAGGCGGGGCAGCCCAAU |
| | | GCCAUGGGCCUGAUCUCACCGCUGCCUCCUUCCC |
| hsa-miR-4732 | 250 | GAGGGAGCUGUAGAGCAGGGAGCAGGAAGCUGUGUGUGUC |
| | | CAGCCCUGACCUGUCCUGUUCUGCCCCCAGCCCCUC |
| hsa-miR-4800 | 251 | GGAGAAAGGAGUGGACCGAGGAAGGAAGGAAGGCAAGGCU |
| | | GUCUGUCCAUCCGUCCGUCUGUCCACCUACCUGUCAGUCC |
| hsa-miR-487b | 252 | UUGGUACUUGGAGAGUGGUUAUCCCUGUCCUGUUCGUUUU |
| | | GCUCAUGUCGAAUCGUACAGGGUCAUCCACUUUUUCAGUA |
| | | UCAA |
| hsa-miR-497 | 253 | CCACCCCGGUCCUGCUCCCGCCCCAGCAGCACACUGUGGU |
| | | UUGUACGGCACUGUGGCCACGUCCAAACCACACUGUGGUG |
| | | UUAGAGCGAGGGUGGGGGAGGCACCGCCGAGG |
| hsa-miR-5001 | 254 | AGCUCAGGGCGGCUGCGCAGAGGGCUGGACUCAGCGGCGG |
| | | AGCUGGCUGCUGGCCUCAGUUCUGCCUCUGUCCAGGUCCU |
| | | UGUGACCCGCCCGCUCUCCU |
| hsa-miR-5010 | 255 | GAUCCAGGGAACCCUAGAGCAGGGGGAUGGCAGAGCAAAA |
| | | UUCAUGGCCUACAGCUGCCUCUUGCCAAACUGCACUGGAU |
| | | UUUGUGUCUCCCAUUCCCCAGAGCUGUCUGAGGUGCUUUG |
| hsa-miR-503 | 256 | UGCCCUAGCAGCGGGAACAGUUCUGCAGUGAGCGAUCGGU |
| | | GCUCUGGGGUAUUGUUUCCGCUGCCAGGGUA |
| hsa-miR-504 | 257 | GCUGCUGUUGGGAGACCCUGGUCUGCACUCUAUCUGUAUU |
| | | CUUACUGAAGGGAGUGCAGGGCAGGGUUUCCCAUACAGAG |
| | | GGC |
| hsa-miR-505 | 258 | GAUGCACCCAGUGGGGGAGCCAGGAAGUAUUGAUGUUUCU |
| | | GCCAGUUUAGCGUCAACACUUGCUGGUUUCCUCUCUGGAG |
| | | CAUC |
| hsa-miR-5100 | 259 | CCAUGAGGAGCUGGCAGUGGGAUGGCCUGGGGGUAGGAGC |
| | | GUGGCUUCUGGAGCUAGACCACAUGGGUUCAGAUCCCAGC |
| | | GGUGCCUCUAACUGGCCACAGGACCUUGGGCAGUCAGCU |
| hsa-miR-532 | 260 | CGACUUGCUUUCUCUCCUCCAUGCCUUGAGUGUAGGACCG |
| | | UUGGCAUCUUAAUUACCCUCCCACACCCAAGGCUUGCAAA |
| | | AAAGCGAGCCU |
| hsa-miR-542 | 261 | CAGAUCUCAGACAUCUCGGGGAUCAUCAUGUCACGAGAUA |
| | | CCAGUGUGCACUUGUGACAGAUUGAUAACUGAAAGGUCUG |
| | | GGAGCCACUCAUCUUCA |
| hsa-miR-5684 | 262 | GCUGAACUCUAGCCUGAGCAACAGAGUGAGAUGGUCUUGU |
| | | UUUGUUGCCCAGGCUGGAGUCCAGU |
| hsa-miR-584 | 263 | UAGGGUGACCAGCCAUUAUGGUUUGCCUGGGACUGAGGAA |
| | | UUUGCUGGGAUAUGUCAGUUCCAGGCCAACCAGGCUGGUU |
| | | GGUCUCCCUGAAGCAAC |
| hsa-miR-625 | 264 | AGGGUAGAGGGAUGAGGGGGAAAGUUCUAUAGUCCUGUAA |
| | | UUAGAUCUCAGGACUAUAGAACUUUCCCCCUCAUCCCUCU |
| | | GCCCU |
| hsa-miR-629 | 265 | UCCCUUUCCCAGGGGAGGGGCUGGGUUUACGUUGGGAGAA |
| | | CUUUUACGGUGAACCAGGAGGUUCUCCCAACGUAAGCCCA |
| | | GCCCCUCCCCUCUGCCU |
| hsa-miR-6511a | 266 | CCUGCAGGCAGAAGUGGGGCUGACAGGGCAGAGGGUUGCG |
| | | CCCCCUCACCAUCCCUUCUGCCUGCAG |
| hsa-miR-652 | 267 | ACGAAUGGCUAUGCACUGCACAACCCUAGGAGAGGGUGCC |
| | | AUUCACAUAGACUAUAAUUGAAUGGCGCCACUAGGGUUGU |
| | | GCAGUGCACAACCUACAC |
| hsa-miR-664b | 268 | UGGGCUAAGGGAGAUGAUUGGGUAGAAAGUAUUAUUCUAU |
| | | UCAUUUGCCUCCCAGCCUACA |
| hsa-miR-671 | 269 | GCAGGUGAACUGGCAGGCCAGGAAGAGGAGGAAGCCCUGG |
| | | AGGGGCUGGAGGUGAUGGAUGUUUUCCUCCGGUUCUCAGG |
| | | GCUCCACCUCUUUCGGGCCGUAGAGCCAGGGCUGGUGC |
| hsa-miR-6723 | 270 | AUGCAUCGGGAUAGUCCGAGUAACGUCGGGGCAUUCCGGA |
| | | UAGGCCGAGAAAGUGUUGUGGGAAGAAAGUUAGAUUUACG |
| | | CCGAUGAAU |
| hsa-miR-708 | 271 | AACUGCCCUCAAGGAGCUUACAAUCUAGCUGGGGGUAAAU |
| | | GACUUGCACAUGAACACAACUAGACUGUGAGCUUCUAGAG |
| | | GGCAGGGA |
| hsa-miR-744 | 272 | UUGGGCAAGGUGCGGGGCUAGGGCUAACAGCAGUCUUACU |
| | | GAAGGUUUCCUGGAAACCACGCACAUGCUGUUGCCACUAA |
| | | CCUCAACCUUACUCGGUC |
| hsa-miR-769 | 273 | GCCUUGGUGCUGAUUCCUGGGCUCUGACCUGAGACCUCUG |
| | | GGUUCUGAGCUGUGAUGUUGCUCUCGAGCUGGGAUCUCCG |
| | | GGGUCUUGGUUCAGGGCCGGGGCCUCUGGGUUCCAAGC |
| hsa-miR-9-1 | 274 | CGGGGUUGGUUGUUAUCUUUGGUUAUCUAGCUGUAUGAGU |
| | | GGUGUGGAGUCUUCAUAAAGCUAGAUAACCGAAAGUAAAA |
| | | AUAACCCCA |
| hsa-miR-9-2 | 275 | GGAAGCGAGUUGUUAUCUUUGGUUAUCUAGCUGUAUGAGU |
| | | GUAUUGGUCUUCAUAAAGCUAGAUAACCGAAAGUAAAAAC |
| | | UCCUUCA |
| hsa-miR-9-3 | 276 | GGAGGCCCGUUUCUCUCUUUGGUUAUCUAGCUGUAUGAGU |
| | | GCCACAGAGCCGUCAUAAAGCUAGAUAACCGAAAGUAGAA |
| | | AUGAUUCUCA |
| hsa-miR-99a | 277 | CCCAUUGGCAUAAACCCGUAGAUCCGAUCUUGUGGUGAAG |
| | | UGGACCGCACAAGCUCGCUUCUAUGGGUCUGUGUCAGUGU |
| | | G |
| hsa-miR-99b | 278 | GGCACCCACCCGUAGAACCGACCUUGCGGGGCCUUCGCCG |
| | | CACACAAGCUCGUGUCUGUGGGUCCGUGUC |
| MID-00206 | 279 | GGGUACAGGUGCACAACGUGCAGGUUUGUUACAUAUGUAU |
| | | ACAUAUGCCAUGUUGGUGUGCUGCACCC |
| MID-00612 | 280 | UCGAGGCUAGAGUCACGCUUGGGUAUCGGCUAUUGCCUGA |
| | | GUGUGCUAGAGUCCUCGA |
| MID-00689 | 281 | GAGUCACAGUGGACUUGGAGUCAGGAGGCCUGAGGUCCUU |
| | | GAAGACCUCCCUGACCUGCUCUGGUCCACUGUGUGCUC |
| MID-00713 | 282 | CUUUGCCGAGACUAGAGUCACAUCCUGACACAACUCUUGU |
| | | CCUGGUGUGCUAGAGUACUCGAAGAG |
| MID-00745 | 283 | UUAUACUUUAAGUUUUAGGGUACAUGUGCACAACGUGCAG |
| | | GUUAGUUACAUAUGUAUACUUGUGCCAUGUUGGUGUGCUG |
| | | CACCCAUUAACUUGUCGUUUAA |
| MID-01140 | 284 | UCCUACCCGGGUCGGAGUUAGCUCAAGCGGUUACCUCCUC |
| | | AUGCCGGACUUUCUAUCUGUCCAUCUCUGUGCUGGGGUUC |
| | | GAGACCCGCGGGUGCUUACUGACCCUUUUAUGCA |
| MID-01141 | 285 | UCCUACCCGGGUCGGAGUUAGCUCAAGCGGUUACCUCCUC |
| | | AUGCCGGACUUUCUAUCUGUCCAUCUCUGUGCUGGGGUUC |
| | | GAGACCCGCGGGUGCUUACUGACCCUUUUAUGCA |
| MID-14865 | 286 | AUUGAUCAUCGACACUUCGAACGCAAUUGCAGCCCGGGUU |
| | | CCUCCCAGGGCUUUGCCUGUCUGAGCGUCGCUUGCCGAUC |
| | | AGUAG |
| MID-15867 | 287 | UGUUUGCUCACCUGUCAUUUCAGCUUUGUGACAAGCCUGA |
| | | CAUGAAAAGGGGAGAGGGCA |
| MID-16318 | 288 | CUGCCAAAGAGCAGCAAGAUGAGCUGGUUUGAUGGGGAGC |
| | | CAUCCCUUGAUGAGGAGAACCCUUCCCACUCUCACUCAGC |
| | | CUCACCCAGCUGCCCUGAGGCAG |
| MID-16752 | 289 | GUGACUCUGGGAGGUGUCGGACGAGCCCCCUGAGGUCUGU |
| | | CUGUGGGCAACGCCUCCCAAAGUGUCAU |
| MID-17144 | 290 | GCUCAGAAGUGAUGAAUUGAUCAGAUAGACGAGGCCGGGC |
| | | UUGUCCCCGGCCACUGAUUAUCGAGGCGAUUCUGAUCUGG |
| | | GC |
| MID-18307 | 291 | GUAGGCCUGGGUCUUGGAUAAACUGAAAGCUGAGCGUGAA |
| | | CGUGGUAUCACCAUUAAUACCUCCUUGUGGAAAUUUGAGA |
| | | ACAGCAAGUGCUAC |
| MID-18336 | 292 | ACCCUACUCCUGGUACCAAUUUACUGUAUUAGUCUGUUUU |
| | | CAUGCUGCUGAUAGAGACAUACCCAGGAGUGGGU |
| MID 18468 | 293 | GACGUGAGGGGGUGCUACAUACAGCAGCUGUGUGUAGUAU |
| | | GUGCCUUUCUCUGUU |
| MID-19149 | 294 | UGGUUGGCCGGUCCAGCGCCUCCAGGAAGUCGAAGCUGCC |
| | | GCAGUGGCGCUUGUUGAAAGGCGCGGGCUCAGCAGGCCGA |
| | | GCCA |
| MID-19433 | 295 | UAGGAAUUCUGGACCAGGCUUAAAAGACUGGGAUGAGGCU |
| | | GGUCCGAAGGUAGUGAGUUAUCUCCAUUGAUAGUUCAGUC |
| | | UGUAACAGAUCAAACUCCUUGUUCUACUCUUUUUUUUUUU |
| | | UUUAGACAGA |
| MID-19434 | 296 | UGGGCUGGUCCGAGUGCAGUGGUGUUUACAAGUAUUUGAU |
| | | UAUAACUAGUUACAGAUUUCUUUGUUUCCUUCUCCACUCC |
| | | CACUGCCUCACUUGACUGGCCUA |
| MID-19898 | 297 | UGGUGGUAUUGGUAGUGUUCCUGUUGGCCGAGUGGAGACU |
| | | GGUGUUCUCAAACCUGGUAUGGUGGUCACCUUUGCUCCAG |
| | | UCAACGUUACAACAGAAGUAAAAUCUGUCGAAAUGCACCA |
| MID-23291 | 298 | CUAAUGGAUAAGGCAUUGGCCUCCUAAGCCAGGGAUUGUG |
| | | GGUUCGAGUCCCAUCUGGGGUGGCCUGUGACUUUUGUCCU |
| | | UUUUUCCCC |
| MID-24702 | 299 | AGUUGGUCCGAGUGUUGUGGGUUAUUGUUAAGUUGAUUUA |
| | | ACAUUGUCUCCCCCCACAACCGCGCUUGACUAGCU |

miR name is the miRBase registry name (release 20), except for the miR names represented by MID-numeral.
miRs represented by MID-numeral were predicted and/or cloned at Rosetta Genomics.

The present inventors have pioneered in the demonstration of differential expression of microRNAs in human glioblastoma sub-populations. As shown herein in Example 7, glioblastoma multiforme (GBM) long-term and short-term survivors showed the expression of different sets of microRNAs. The two sub-populations provided herein were the most different in terms of survival time, while other clinical parameters remained similar, such as for example tumor location and percentage of tumor removal (post-surgery). The identification of the differential microRNA expression in the sub-populations serves for the identification of the most-likely candidates to be selected for GBM therapy, be it in the form of mimetics or anti-miR. A list of microRNAs that presented a fold-change of 2, and which may be singled out as candidates for use in therapeutics are hsa-miR-99a-5p (SEQ ID NO.123), hsa-miR-129-2-3p (SEQ ID NO.14), hsa-miR-708-5p (SEQ ID NO.117), hsa-miR-34c-5p (SEQ ID NO.65), hsa-miR-374b-5p (SEQ ID NO.70), hsa-miR-99a-3p (SEQ ID NO.122), hsa-miR-195-5p (SEQ ID NO.32), hsa-miR-34b-5p (SEQ ID NO.64), hsa-miR-34a-5p (SEQ ID NO.63), hsa-miR-155-5p (SEQ ID NO.24), hsa-miR-584-5p (SEQ ID NO.108), MID-01140 (SEQ ID NO.130), MID-01141 (SEQ ID NO.131), hsa-miR-34a-3p (SEQ ID NO.62), hsa-miR-1290 (SEQ ID NO.15), hsa-miR-4324 (SEQ ID NO.84) and hsa-miR-374c-5p (SEQ ID NO.71).

Other miRs which have been shown in the literature to be overexpressed in GBM are hsa-miR-17-3p, hsa-miR-17-5p, hsa-miR-19a, hsa-miR-20a, hsa-miR-92a, hsa-miR-21 and hsa-miR-93. Other miRs which have been shown to be downregulated in GBM include hsa-miR-7, hsa-miR-128 and hsa-miR-137 [Moller et al., (2013) Mol. Neurobiol. Vol. 47, p.131-144].

Thus, nucleic acids comprised in the system of the invention are provided herein. The nucleic acid may comprise the sequence of SEQ ID NOS: 1-299 or variants thereof. The variant may be a perfect or imperfect complement of the referenced nucleotide sequence. Alternatively, the variant may be a nucleotide sequence that is substantially identical to the referenced nucleotide sequence or the complement thereof. The variant may also be a nucleotide sequence which hybridizes under stringent conditions to the referenced nucleotide sequence, complements thereof (like an anti-miR sequence complement to the miRNA), or nucleotide sequences substantially identical thereto.

The nucleic acid may have a length of 10 to 530 nucleotides. The nucleic acid may have a length of at least 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 125, 150, 175, 200, 250 or 530 nucleotides. The nucleic acid may be synthesized or expressed in a cell (in vitro or in vivo) using a synthetic gene described herein. The nucleic acid may be synthesized as a single strand molecule and hybridized to a substantially complementary nucleic acid to form a duplex. The nucleic acid may be introduced to a cell, tissue or organ in a single- or double-stranded form or capable of being expressed by a synthetic gene using methods well known to those skilled in the art, including as described in U.S. Pat. No. 6,506,559 which is incorporated herein by reference.

The nucleic acid may comprise a sequence of a pri-miRNA or a variant thereof. The pri-miRNA sequence may comprise from 45-30,000, 50-25,000, 100-20,000, 1,000-1,500, 500-750, or 80-100 nucleotides. The sequence of the pri-miRNA may comprise a pre-miRNA, miRNA and miRNA*, as set forth herein, and variants thereof. The sequence of the pri-miRNA may comprise the sequence of SEQ ID NOs: 1-145 and 152-299 or variants thereof. In another particular embodiment, the nucleic acid is a miR comprising any one of the sequences denoted by SEQ ID NO. 1-145.

The pri-miRNA may form a hairpin structure. The hairpin may comprise first and second nucleic acid sequences that are substantially complimentary. The first and second nucleic acid sequence may be from 37-50 nucleotides. The first and second nucleic acid sequence may be separated by a third sequence of from 8-12 nucleotides. The hairpin structure may have a free energy less than −25 Kcal/mole as calculated by the Vienna algorithm with default parameters, as described in Hofacker et al., Monatshefte f. Chemie 125: 167-188 (1994), the contents of which are incorporated herein. The hairpin may comprise a terminal loop of 4-20, 8-12 or 10 nucleotides. The pri-miRNA may comprise at least 19% adenosine nucleotides, at least 16% cytosine nucleotides, at least 23% thymine nucleotides and at least 19% guanine nucleotides.

The nucleic acid may also comprise a sequence of a pre-miRNA or a variant thereof. The pre-miRNA sequence may comprise from 45-200, 60-80 or 60-70 nucleotides. The sequence of the pre-miRNA may comprise a miRNA and a miRNA* as set forth herein. The sequence of the pre-miRNA may also be that of a pri-miRNA excluding from 0-160 nucleotides from the 5′ and 3′ ends of the pri-miRNA. The sequence of the pre-miRNA may comprise the sequence of SEQ ID NOS: 1-145 and 152-299 or variants thereof. In one particular embodiment, the sequence of the pre-miRNA may comprise the sequence of SEQ ID NOS: 1-145.

The nucleic acid may also comprise a sequence of a miRNA (including miRNA*) or a variant thereof. The miRNA sequence may comprise from 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA. The sequence of the miRNA may comprise the sequence of SEQ ID NOS: 1-299 or variants thereof. In one particular embodiment, the sequence of the miRNA may comprise the sequence of SEQ ID NOs. 1-145.

The nucleic acid may also comprise a sequence of an anti-miRNA that is capable of blocking the activity of a miRNA or miRNA*, such as by binding to the pri-miRNA, pre-miRNA, miRNA or miRNA* (e.g. antisense or RNA silencing), or by binding to the target binding site. The anti-miRNA may comprise a total of 5-100 or 10-60 nucleotides. The anti-miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the anti-miRNA may comprise (a) at least 5 nucleotides that are substantially identical or complimentary to the 5′ of a miRNA and at least 5-12 nucleotides that are substantially complimentary to the flanking regions of the target site from the 5′ end of the miRNA, or (b) at least 5-12 nucleotides that are substantially identical or complimentary to the 3′ of a miRNA and at least 5 nucleotide that are substantially complimentary to the flanking region of the target site from the 3′ end of the miRNA. The sequence of the anti-miRNA may comprise the compliment of SEQ ID NOs: 1-145 or variants thereof.

In another aspect, the present invention provides a pharmaceutical composition comprising as active agent the system as defined herein, said system comprising a nanocarrier and a nucleic acid. In one particular embodiment, said nanocarrier comprises a compound having a structure according to formula (I) as hereinbefore described, and the nucleic acid is a microRNA. Said pharmaceutical composition further comprising any one of adjuvants, carriers, diluents and excipients.

In another particular embodiment of the pharmaceutical composition of the invention, said nucleic acid comprises a sequence denoted by any one of hsa-miR-99a-5p (SEQ ID NO.123), hsa-miR-129-2-3p (SEQ ID NO.14), hsa-miR-708-5p (SEQ ID NO.117), hsa-miR-34c-5p (SEQ ID NO.65), hsa-miR-374b-5p (SEQ ID NO.70), hsa-miR-99a-3p (SEQ ID NO.122), hsa-miR-195-5p (SEQ ID NO.32), hsa-miR-34b-5p (SEQ ID NO.64), hsa-miR-34a-5p (SEQ ID NO.63), hsa-miR-155-5p (SEQ ID NO.24), hsa-miR-584-5p (SEQ ID NO.108), MID-01140 (SEQ ID NO.130), MID-01141 (SEQ ID NO.131), hsa-miR-34a-3p (SEQ ID NO.62), hsa-miR-1290 (SEQ ID NO.15), hsa-miR-4324 (SEQ ID NO.84) and hsa-miR-374c-5p (SEQ ID NO.71), or a complement thereof, or a sequence at least about 80%, or at least about 85%, or at least about 90%, or at least about 95% identical to hsa-miR-99a-5p (SEQ ID NO.123), hsa-miR-129-2-3p (SEQ ID NO.14), hsa-miR-708-5p (SEQ ID NO.117), hsa-miR-34c-5p (SEQ ID NO.65), hsa-miR-374b-5p (SEQ ID NO.70), hsa-miR-99a-3p (SEQ ID NO.122), hsa-miR-195-5p (SEQ ID NO.32), hsa-miR-34b-5p (SEQ ID NO.64), hsa-miR-34a-5p (SEQ ID NO.63), hsa-miR-155-5p (SEQ ID NO.24), hsa-miR-584-5p (SEQ ID NO.108), MID-01140 (SEQ ID NO.130), MID-01141 (SEQ ID NO.131), hsa-miR-34a-3p (SEQ ID NO.62), hsa-miR-1290 (SEQ ID NO.15), hsa-miR-4324 (SEQ ID NO.84) and hsa-miR-374c-5p (SEQ ID NO.71), or a complement thereof.

In one more particular embodiment of the pharmaceutical composition of the invention, said RNA is hsa-miR-34a, denoted by SEQ ID NO:63, or a sequence at least about 80%, 85%, 90% or 95% identical to SEQ ID NO:63.

In some of these embodiments of the pharmaceutical composition of the invention, the nanocarrier is as described in any of the embodiments described herein.

Thus, the system of the invention per se, or comprised in a pharmaceutical composition or medicament, may be utilized to transport a microRNA entity, mimetic or anti-miR, into at least one prokaryotic or eukaryotic cell, in particular into at least one human or animal cell. Transporting said microRNA into a plurality of cells is preferred. Suited animal cells are, e.g., cells of mammals like, e.g., humans or rodents such as rats or mice.

In an alternative embodiment, the system of the invention is used to transport microRNAs into at least one animal cell but not into a human cell. Thus, the use of system may be defined as for in vitro, in vivo, ex vivo or in situ applications with respect to animal cells and for in vitro, ex vivo or in situ applications for human cells.

In another alternative embodiment, the system of the invention is used to transport microRNAs into a human cell, tissue or organ, in vivo or ex vivo.

The pharmaceutical composition may comprise the system described herein and optionally a pharmaceutically acceptable carrier. The composition may encompass modified oligonucleotides that are identical, substantially identical, substantially complementary or complementary to any nucleobase sequence version of the miRNAs described herein or a precursor thereof.

In certain embodiments, a nucleobase sequence of a modified oligonucleotide is fully identical or complementary to a microRNA nucleobase sequence listed herein, or a precursor thereof. In certain embodiments, a modified oligonucleotide has a nucleobase sequence having one mismatch with respect to the nucleobase sequence of the mature microRNA, or a precursor thereof. In certain embodiments, a modified oligonucleotide has a nucleobase sequence having two mismatches with respect to the nucleobase sequence of the microRNA, or a precursor thereof. In certain such embodiments, a modified oligonucleotide has a nucleobase sequence having no more than two mismatches with respect to the nucleobase sequence of the mature microRNA, or a precursor thereof. In certain such embodiments, the mismatched nucleobases are contiguous. In certain such embodiments, the mismatched nucleobases are not contiguous.

In certain embodiments, a modified oligonucleotide consists of a number of linked nucleosides that is equal to the length of the mature microRNA.

In certain embodiments, the number of linked nucleosides of a modified oligonucleotide is less than the length of the mature microRNA. In certain such embodiments, the number of linked nucleosides of a modified oligonucleotide is one less than the length of the mature miRNA. In certain such embodiments, a modified oligonucleotide has one less nucleoside at the 5′ terminus. In certain such embodiments, a modified oligonucleotide has one less nucleoside at the 3′ terminus. In certain such embodiments, a modified oligonucleotide has two fewer nucleosides at the 5′ terminus. In certain such embodiments, a modified oligonucleotide has two fewer nucleosides at the 3′ terminus. A modified oligonucleotide having a number of linked nucleosides that is less than the length of the miRNA, wherein each nucleobase of a modified oligonucleotide is complementary to each nucleobase at a corresponding position in a miRNA, is considered to be a modified oligonucleotide having a nucleobase sequence that is fully complementary to a portion of a miRNA sequence.

In certain embodiments, a modified oligonucleotide consists of 15 to 30 linked nucleosides. In certain embodiments, a modified oligonucleotide consists of 19 to 24 linked nucleosides. In certain embodiments, a modified oligonucleotide consists of 21 to 24 linked nucleosides. In certain embodiments, a modified oligonucleotide consists of 15 linked nucleosides. In certain embodiments, a modified oligonucleotide consists of 16 linked nucleosides. In certain embodiments, a modified oligonucleotide consists of 17 linked nucleosides. In certain embodiments, a modified oligonucleotide consists of 18 linked nucleosides. In certain embodiments, a modified oligonucleotide consists of 19 linked nucleosides. In certain embodiments, a modified oligonucleotide consists of 20 linked nucleosides. In certain embodiments, a modified oligonucleotide consists of 21 linked nucleosides. In certain embodiments, a modified oligonucleotide consists of 22 linked nucleosides. In certain embodiments, a modified oligonucleotide consists of 23 linked nucleosides. In certain embodiments, a modified oligonucleotide consists of 24 linked nucleosides. In certain embodiments, a modified oligonucleotide consists of 25 linked nucleosides. In certain embodiments, a modified oligonucleotide consists of 26 linked nucleosides. In certain embodiments, a modified oligonucleotide consists of 27 linked nucleosides. In certain embodiments, a modified oligonucleotide consists of 28 linked nucleosides. In certain embodiments, a modified oligonucleotide consists of 29 linked nucleosides. In certain embodiments, a modified oligonucleotide consists of 30 linked nucleosides.

Modified oligonucleotides of the present invention may comprise one or more modifications to a nucleobase, sugar, and/or internucleoside linkage. A modified nucleobase, sugar, and/or internucleoside linkage may be selected over an unmodified form because of desirable properties such as, for example, enhanced cellular uptake, enhanced affinity for other oligonucleotides or nucleic acid targets and increased stability in the presence of nucleases.

In certain embodiments, a modified oligonucleotide of the present invention comprises one or more modified nucleosides. In certain such embodiments, a modified nucleoside is a stabilizing nucleoside. An example of a stabilizing nucleoside is a sugar-modified nucleoside. The miRNA molecules may be designed to resist degradation by modifying it to include phosphorothioate, or other linkages, methylphosphonate, sulfone, sulfate, ketyl, phosphorodithioate, phosphoramidate, phosphate esters, and the like. Modifications designed to increase in vivo stability include, but are not limited to, the addition of flanking sequences at the 5′ and/or 3′ ends; the use of phosphorothioate or 2′ O-methyl rather than phosphodiester linkages in the backbone; and/or the inclusion of non-traditional bases such as inosine, queosine, and wybutosine and the like, as well as acetyl- methyl-, thio- and other modified forms of adenine, cytidine, guanine, thymine, and uridine. In addition, chemically synthesizing nucleic acid molecules with modifications (base, sugar and/or phosphate) can prevent their degradation by serum ribonucleases, which can increase their potency.

In certain embodiments, a modified nucleoside is a sugar-modified nucleoside. In certain such embodiments, the sugar-modified nucleosides can further comprise a natural or modified heterocyclic base moiety and/or a natural or modified internucleoside linkage and may include further modifications independent from the sugar modification. In certain embodiments, a sugar modified nucleoside is a 2′-modified nucleoside, wherein the sugar ring is modified at the 2′ carbon from natural ribose or 2′-deoxy-ribose. In certain embodiments, 2′-O-methyl group is present in the sugar residue. The 2′-O-methyl modification is advantageous in the synthesis of RNA molecules in that it makes it nuclease resistant. In addition, 2′-O-methyl modified molecules form stable hybrids with RNA.

The nucleic acid comprised in the system of the invention may thus have a 2′-O-methyl group in the 5′ and/or in the 3′ end, and/or in any other nucleotide, not necessarily in the extremities.

The following are examples of nucleic acid molecules having a 2′-O-methyl modification (represented by the underline):

hsa-miR-34a-5p
(SEQ ID NO. 63)
UGGCAGUGUCUUAGCUGGUUGU
hsa-miR-34a-3p
(SEQ ID NO. 62)
CAAUCAGCAAGUAUACUGCCCU
NC (guide)
(SEQ ID NO. 146)
UGGACUCUGAGAAAGGAGUAUG
NC (passenger)
(SEQ ID NO. 147)
UACUCCUUAUCAGACUCCAUA

In certain embodiments the nucleic acid may also be provided as a conjugate. Such conjugate (and/or complex) may be used to facilitate delivery of microRNA molecules into a biological system, such as a cell. Conjugates and complexes can impart therapeutic activity by transferring therapeutic compounds across cellular membranes, altering the pharmacokinetics, and/or modulating the localization of nucleic acid molecules delivered by the nanocarrier system of the invention. Such conjugates are known in the art, and include, but are not limited, to small molecules, lipids, cholesterol, phospholipids, negatively charged polymers and other polymers, proteins, peptides, hormones, carbohydrates, and polysaccharides, which may be conjugated or complexed to the nucleic acid comprised in the nanocarrier system described herein.

The nucleotide sequences designed according to the teachings of the present invention can be generated according to any nucleotide synthesis method known in the art, including both enzymatic and solid-phase synthesis. Equipment and reagents for executing solid-phase synthesis are commercially available from, for example, Applied Biosystems. Any other means for such synthesis may also be employed; the actual synthesis of the nucleotides is well within the capabilities of one skilled in the art and can be accomplished via established methodologies as detailed in, for example: Sambrook, J. and Russell, D. W. (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Md.; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.

It will be appreciated that the nucleic acid molecule in complex with the nanocarrier system of the invention may also be generated using an expression vector as known in the art.

Thus, the nucleic acid comprised in the system of the invention may be generated according to any nucleotide synthesis method known in the art, therefore generating a synthetic microRNA, it being a mimetic microRNA, or alternatively, an anti-microRNA.

Thus, in one embodiment, the system of the invention comprises a nanocarrier as described herein and a synthetic nucleic acid, duplex or single-stranded.

The synthetic nucleic acid may have modifications at the 5′- and/or at the 3′-end. Alternatively, the synthetic nucleic acid may have modified nucleotides within the molecule.

Synthetic nucleic acids comprising a 2′-O-methyl modification may be denoted, e.g. as follows (the nucleotide having the modification is marked by an underline):

hsa-miR-34a-5p-2′-O-Me (SEQ ID NO. 148):
UGGCAGUGUCUUAGCUGGUUGU
hsa-miR-34a-3p-2′-O-Me (SEQ ID NO. 149):
CAAUCAGCAAGUAUACUGCCCU
NC-5p-2′-O-Me (SEQ ID NO. 150):
UGGACUCUGAGAAAGGAGUAUG
NC-3p-2′-O-Me (SEQ ID NO. 151):
UACUCCUUAUCAGACUCCAUA

The system provided herein may be used for therapeutic applications. The system may be used for delivering mimetic microRNAs as well as anti-microRNAs.

The delivery of mimetic microRNAs is particularly useful for restoring microRNA expression in diseases in which expression is consistently reduced. microRNA mimetics can be modified to have enhanced efficiency by increasing the affinity for a specific target and by reducing other unwanted microRNA effects.

The delivery of anti-microRNAs is an alternative therapeutic strategy, in which antisense oligonucleotides that bind directly to microRNAs are delivered to the cell and block their activity. The delivery of anti-microRNAs is important for blocking microRNA expression in diseases in which expression is consistently enhanced. The anti-microRNAs work by stoichiometric interaction with mature microRNAs, either titrating them from biologically active pools of mature microRNAs or binding to microRNA precursors and inhibiting the biogenesis of mature microRNAs. Generally, an anti-microRNA is “antisense” to a target nucleic acid (a target miR) when, written in the 5′ to 3′ direction, it comprises the reverse complement of the corresponding region of the miR. In general, “antisense compounds” are also often defined in the art to comprise the further limitation of, once hybridized to a target, being able to induce or trigger a reduction in target gene expression.

In a further embodiment of the invention, the nanocarrier is complexed to perfect complementary microRNA duplexes to improve RISC loading of said microRNAs. In another further embodiment, the microRNA duplexes may comprise from at least one up to five mismatches within the duplex molecule. Thus, the duplex may comprise one, two, three, four or five mismatches.

In another further embodiment of the invention, the nanocarrier is complexed to a single-stranded anti-microRNA which may prevent and/or disturb RISC loading of the corresponding complementary microRNA.

The system or a pharmaceutical composition comprising the system described herein may be administered by known methods, including introducing the system of the invention into a desired target cell in vitro or in vivo.

Several methods for the delivery of nucleic acid molecules have been described, including e.g. Akhtar et al. (Trends Cell Bio. 2, 139, 1992). WO 94/02595 describes general methods for delivery of RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres.

The present invention provides a delivery system in which a microRNA is delivered as a component of a nanocarrier complex as described herein.

The system described herein or a pharmaceutical composition comprising thereof may be locally delivered by direct injection intratumorally or intravenously, by use of an infusion pump, through a cannula, and the like. Other routes of delivery include, but are not limited to oral (tablet or pill form) and/or intrathecal delivery (Gold, 1997, Neuroscience, 76, 1153-1158). Other approaches are provided for example in WO93/23569, WO99/05094, and WO99/04819.

Jet injection may also be used for intra-muscular administration, as described by Furth et al. (Anal Biochem 115 205:365-368, 1992). The system or a pharmaceutical composition comprising thereof may be delivered intradermally by a particle bombardment device, or “gene gun” as described in the literature (see, for example, Tang et al. Nature 356:152-154, 1992), where gold microprojectiles are coated with the system of the invention, then bombarded into skin cells.

The system of the present invention can be formulated into pharmaceutical compositions by combination with appropriate, pharmaceutically acceptable carriers or diluents, and can be formulated into preparations in solid, semi-solid, liquid or gaseous forms, such as tablets, capsules, powders, granules, ointments, solutions, suppositories, injections, inhalants and aerosols. As such, administration of the system or a pharmaceutical composition comprising thereof can be achieved in various ways, including oral, buccal, rectal, parenteral, intraperitoneal, intradermal, transdermal, intratracheal, etc.

In certain embodiments, a pharmaceutical composition of the present invention is administered in the form of a dosage unit (e.g., tablet, capsule, bolus, etc.). In certain embodiments, such pharmaceutical compositions comprise a system in a dose selected from 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 270 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, and 800 mg. In certain such embodiments, a pharmaceutical composition of the present invention comprises a dose of system selected from 25 mg, 50 mg, 75 mg, 100 mg, 150 mg, 200 mg, 250 mg, 300 mg, 350 mg, 400 mg, 500 mg, 600 mg, 700 mg, and 800 mg.

In certain embodiments, a pharmaceutical agent is a sterile lyophilized modified system of the invention that is reconstituted with a suitable diluent, e.g., sterile water for injection or sterile saline for injection. The reconstituted product is administered as a subcutaneous injection or intratumor injection or as an intravenous infusion after dilution into saline. The lyophilized system of the invention consists of a system which has been prepared in water for injection, or in saline for injection, adjusted to pH 7.0-9.0 with acid or base during preparation, and then lyophilized. The lyophilized system may be 25-800 mg of said system. It is understood that this encompasses 25, 50, 75, 100, 125, 150, 175, 200, 225, 250, 275, 300, 325, 350, 375, 425, 450, 475, 500, 525, 550, 575, 600, 625, 650, 675, 700, 725, 750, 775, and 800 mg of lyophilized system.

In certain embodiments, the pharmaceutical compositions comprising the system of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nanocarriers or the microRNAs of the formulation.

In certain embodiments, pharmaceutical compositions of the present invention comprise one or more systems of the invention and one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, a pharmaceutical composition of the present invention is prepared using known techniques, including, but not limited to mixing, dissolving, granulating, dragee-making, levigating, emulsifying, encapsulating, entrapping or tabletting processes.

In certain embodiments, a pharmaceutical composition of the present invention is a liquid (e.g., a suspension, elixir and/or solution). In certain of such embodiments, a liquid pharmaceutical composition is prepared using ingredients known in the art, including, but not limited to, water, glycols, oils, alcohols, flavoring agents, preservatives, and coloring agents.

In certain embodiments, a pharmaceutical composition of the present invention is a solid (e.g., a powder, tablet, and/or capsule). In certain of such embodiments, a solid pharmaceutical composition comprising one or more systems of the invention is prepared using ingredients known in the art, including, but not limited to, starches, sugars, diluents, granulating agents, lubricants, binders, and disintegrating agents.

In certain embodiments, a pharmaceutical composition of the present invention is formulated as a depot preparation. Certain such depot preparations are typically longer acting than non-depot preparations. In certain embodiments, such preparations are administered by implantation (for example subcutaneously or intramuscularly) or by intramuscular injection. In certain embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a pharmaceutical composition of the present invention comprises one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, a pharmaceutical composition of the present invention comprises a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, a pharmaceutical composition of the present invention comprises a sustained-release system. A non-limiting example of such a sustained-release system is a semi-permeable matrix of solid hydrophobic polymers. In certain embodiments, sustained-release systems may, depending on their chemical nature, release pharmaceutical agents over a period of hours, days, weeks or months.

In certain embodiments, a pharmaceutical composition of the present invention is prepared for oral administration. In certain of such embodiments, a pharmaceutical composition is formulated by combining one or more compounds comprising systems with one or more pharmaceutically acceptable carriers. Certain of such carriers enable pharmaceutical compositions to be formulated as tablets, pills, dragees, capsules, liquids, gels, syrups, slurries, suspensions and the like, for oral ingestion by a subject. In certain embodiments, pharmaceutical compositions for oral use are obtained by mixing the system and one or more solid excipient. Suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In certain embodiments, such a mixture is optionally ground and auxiliaries are optionally added. In certain embodiments, pharmaceutical compositions are formed to obtain tablets or dragee cores. In certain embodiments, disintegrating agents (e.g., cross-linked polyvinyl pyrrolidone, agar, or alginic acid or a salt thereof, such as sodium alginate) are added.

In certain embodiments, dragee cores are provided with coatings. In certain such embodiments, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In certain embodiments, pharmaceutical compositions for oral administration are push-fit capsules made of gelatin. Certain of such push-fit capsules comprise one or more pharmaceutical agents of the present invention in admixture with one or more filler such as lactose, binders such as starches, and/or lubricants such as talc or magnesium stearate and, optionally, stabilizers. In certain embodiments, pharmaceutical compositions for oral administration are soft, sealed capsules made of gelatin and a plasticizer, such as glycerol or sorbitol. In certain soft capsules, one or more pharmaceutical agents of the present invention are be dissolved or suspended in suitable liquids, such as fatty oils, liquid paraffin, or liquid polyethylene glycols. In addition, stabilizers may be added.

In certain embodiments, pharmaceutical compositions are prepared for buccal administration. Certain of such pharmaceutical compositions are tablets or lozenges formulated in conventional manner.

In certain embodiments, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks' solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

In certain embodiments, a pharmaceutical composition is prepared for transmucosal administration. In certain of such embodiments penetrants appropriate to the barrier to be permeated are used in the formulation. Such penetrants are generally known in the art.

In certain embodiments, a pharmaceutical composition is prepared for administration by inhalation. Certain of such pharmaceutical compositions for inhalation are prepared in the form of an aerosol spray in a pressurized pack or a nebulizer. Certain of such pharmaceutical compositions comprise a propellant, e.g., dichlorodifluoromethane, trichlorofluoromethane, dichlorotetrafluoroethane, carbon dioxide or other suitable gas. In certain embodiments using a pressurized aerosol, the dosage unit may be determined with a valve that delivers a metered amount. In certain embodiments, capsules and cartridges for use in an inhaler or insufflator may be formulated. Certain of such formulations comprise a powder mixture of a pharmaceutical agent of the invention and a suitable powder base such as lactose or starch.

In certain embodiments, a pharmaceutical composition is prepared for rectal administration, such as a suppositories or retention enema. Certain of such pharmaceutical compositions comprise known ingredients, such as cocoa butter and/or other glycerides.

In certain embodiments, a pharmaceutical composition is prepared for topical administration. Certain of such pharmaceutical compositions comprise bland moisturizing bases, such as ointments or creams. Exemplary suitable ointment bases include, but are not limited to, petrolatum, petrolatum plus volatile silicones, and lanolin and water in oil emulsions. Exemplary suitable cream bases include, but are not limited to, cold cream and hydrophilic ointment.

In certain embodiments, a pharmaceutical composition of the present invention comprises a system in a therapeutically effective amount. In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art.

In certain embodiments, the system of the present invention is formulated as a prodrug. In certain embodiments, upon in vivo administration, a prodrug is chemically converted to the biologically, pharmaceutically or therapeutically more active form of the system of the invention. In certain embodiments, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, prodrugs are less water soluble than the corresponding active form. In certain instances, such prodrugs possess superior transmittal across cell membranes, where water solubility is detrimental to mobility. In certain embodiments, a prodrug is an ester. In certain such embodiments, the ester is metabolically hydrolyzed to carboxylic acid upon administration. In certain instances the carboxylic acid containing compound is the corresponding active form. In certain embodiments, a prodrug comprises a short peptide (polyaminoacid) bound to an acid group. In certain of such embodiments, the peptide is cleaved upon administration to form the corresponding active form.

In certain embodiments, a prodrug is produced by modifying a pharmaceutically active compound such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

In another aspect, the present invention provides a method of treating cancer, said method comprising administering a therapeutically effective amount of the system described herein, or a composition comprising thereof, to a subject in need.

In one particular embodiment, said cancer is a brain tumor.

As referred to herein, a brain tumor is an intracranial solid neoplasm, a tumor (defined as an abnormal growth of cells) within the brain or the central spinal canal. The most common primary brain tumors are gliomas (arise from glial cells), meningiomas (arise in the meninges), pituitary adenomas (occur in the pituitary gland) and nerve sheath tumor (myelin surrounding nerves).

In another particular embodiment, said brain tumor is glioblastoma (GBM).

Thus, the system of the invention is for use in the treatment of cancer. In one embodiment of the invention, said cancer is brain tumor.

In one alternative embodiment, the use is directed only to in vitro, ex vivo or in situ applications, but not to in vivo applications.

In the Examples section below, one form of the system of the invention, comprising a mimetic hsa-miR-34a-5p (SEQ ID NO.63) is presented. In particular examples presented herein, the PG-NH₂-miR-34a polyplex or the FS-157-miR-34a system are capable of inhibiting cell proliferation, cell cycle progression, and cell migration, inhibiting tumor growth, increasing survival time during disease, and activating targets. The system of the invention also showed to affect miR targets such as c-Met and Notch1, and inhibit their expression. Notch1 is a transmembrane receptor which plays a role in developmental processes, such as promoting the differentiation of progenitor cells into astroglia. MET protein is a membrane receptor that is essential for embryonic development and wound healing.

Thus, in a further aspect, the present invention provides a system comprising a nanocarrier and a nucleic acid comprising a sequence denoted by SEQ ID NO.1-299 or a variant or a complementary thereof, as described herein, for use in the inhibition of cell proliferation, which may also be referred to as cell growth inhibition.

In another further aspect the present invention provides a system comprising a nanocarrier and a nucleic acid comprising a sequence denoted by SEQ ID NO.1-299, a variant or a complementary sequence thereof, as described herein, for use in inhibition of cell cycle progression. Alternatively, the system of the invention may be used for S1 phase arrest.

In another further aspect, the present invention provides a system comprising a nanocarrier and a nucleic acid comprising a sequence denoted by SEQ ID NO.1-299, a variant or a complementary sequence thereof, as described herein, for the inhibition of cell migration.

In another further aspect, the present invention provides a system comprising a nanocarrier and a nucleic acid comprising a sequence denoted by SEQ ID NO.1-299, a variant or a complementary sequence thereof, as described herein, for the inhibition of c-Met and/or Notch1 expression.

In another further aspect, the present invention provides a system comprising a nanocarrier and a nucleic acid comprising a sequence denoted by SEQ ID NO.1-299, a variant or a complementary sequence thereof, as described herein, for the inhibition of tumor growth or tumor progression.

The intra-cellular mechanism of action of therapeutic mimetic miRNAs or anti-miRNAs has not been fully characterized, but gene silencing has been proposed as one such mechanism, wherein the gene to be silenced is e.g. a tumor-related gene. It is possible that the miRNA comprised in the system of the invention interacts with mRNA present in said cell.

In another alternative embodiment, the use is directed only to in vitro or ex vivo applications, but not to in vivo or in situ applications.

The miR-PG-NH₂ polyplexes presented herein are a novel therapeutic entity which either replaces the activity of the natural miR, in the case of mimetic miR-PG-NH₂ polyplexes, or inhibits the activity of the natural miR, in the case of anti-miR-NH₂ polyplexes.

hsa-miR-34a was shown to have tumor suppressor activity (WO 2008/104974) and its replacement in cancers has a great therapeutic value. The system presented herein exhibits an improved performance compared to that of naked hsa-miR-34a.

The absence or downregulation of hsa-miR-34a is especially relevant in p53 negative tumors, since hsa-miR-34 has been demonstrated to be a downstream target of p53. p53-negative tumors are tumors in which there is partial or total loss of p53 function. Generally, mutant p53 protein may still accumulate in the cell. Hence, the PG-NH₂-miR34a or the FS-157-miR-34a polyplexes of the invention are considerably relevant for the treatment of secondary GBMs which are characterized by functional loss of TP53, mainly caused by gene mutations and partial or complete loss of chromosome 10q (secondary GBMs are the result of progression from lower grade astrocytomas).

Cancer treatments often comprise more than one therapy. Thus, the system of the present invention, or a pharmaceutical composition or a medicament comprising thereof, may be optionally further combined with a chemotherapeutic agent, a combination of chemotherapeutic agents and/or radiotherapy. The system of the present invention, or the pharmaceutical composition or medicament comprising thereof, may be optionally further combined with any adjuvant therapy.

In another further embodiment, the present invention provides methods for treating cancer comprising administering to a subject in need thereof the system of the present invention, or a pharmaceutical composition or a medicament comprising thereof, and further optionally comprising administering at least one additional therapy.

In certain embodiments, an additional therapy may be a chemotherapeutic agent. Suitable chemotherapeutic agents include 5-fluorouracil, gemcitabine, doxorubicine, daunorubicin, taxanes like paclitaxel Taxol™, docetaxel; vinca alkaloids like vincristine and vinblastine, anti-metabolites like methotrexate, 5-fluorouracil (5 FU), leucovorin, mitomycin c, sorafenib, etoposide, carboplatin, epirubicin, irinotecan, idarubicin, raltitrexed, tamoxifen and cisplatin, carboplatin, actinomycin D, mitoxantrone or blenoxane or mithramycin, and oxaliplatin. An additional therapy may be surgical resection of tumor(s), radiotherapy or chemoembolization.

In another further aspect a kit is provided. The components of said kit include any one or all of the following: the miR-PG-NH₂ polyplex of the invention, means for diluting the polyplex in case it is in lyophilized form, such as saline, and means for administering the miR-PG-NH₂ polyplex of the invention. As used herein, “means for administering” the polyplex system of the invention refers to a syringe and needle or equivalent, a pump, a catheter, a cannula, tubing for infusion, and the like. The kit provided herein may be used for cancer treatment, for inhibition of cell proliferation or cell migration, for the inhibition of tumor growth, or for the induction or inhibition of microRNA targets.

The following are definitions of terms used herein.

As used herein, the term “aberrant proliferation” means cell proliferation that deviates from the normal, proper, or expected course. For example, aberrant cell proliferation may include inappropriate proliferation of cells whose DNA or other cellular components have become damaged or defective. Aberrant cell proliferation may include cell proliferation whose characteristics are associated with an indication caused by, mediated by, or resulting in inappropriately high levels of cell division, inappropriately low levels of cell death, or both. Such indications may be characterized, for example, by single or multiple local abnormal proliferations of cells, groups of cells, or tissue(s), whether cancerous or non-cancerous, benign or malignant.

As used herein, the term “about” refers to +/−10%.

“Acceptable safety profile” means a pattern of side effects that is within clinically acceptable limits.

“Administering” means providing a pharmaceutical agent or composition to a subject, and includes, but is not limited to, administering by a medical professional and self-administering.

“Parenteral administration,” means administration through injection or infusion. Parenteral administration includes, but is not limited to, subcutaneous administration, intravenous administration, or intramuscular administration.

“Subcutaneous administration” means administration just below the skin.

“Intravenous administration” means administration into a vein.

“Intratumoral administration” means administration within a tumor.

“Chemoembolization” means a procedure in which the blood supply to a tumor is blocked surgically or mechanically and chemotherapeutic agents are administered directly into the tumor.

The term “amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

The term “antisense,” as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription or translation. In this manner, mutant phenotypes may be generated.

“Apoptosis” as used herein, refers to a form of cell death that includes progressive contraction of cell volume with the preservation of the integrity of cytoplasmic organelles; condensation of chromatin (i.e., nuclear condensation), as viewed by light or electron microscopy; and/or DNA cleavage into nucleosome-sized fragments, as determined by centrifuged sedimentation assays. Apoptosis occurs when the membrane integrity of the cell is lost (e.g., membrane blebbing) with engulfment of intact cell fragments (“apoptotic bodies”) by phagocytic cells.

The term “cancer” is meant to include all types of cancerous growths or oncogenic processes, metastatic tissues or malignantly transformed cells, tissues, or organs, irrespective of histopathologic type or stage of invasiveness. Examples of cancers include but are not limited to solid tumors and leukemias, including: apudoma, choristoma, branchioma, malignant carcinoid syndrome, carcinoid heart disease, carcinoma (e.g., Walker, basal cell, basosquamous, Brown-Pearce, ductal, Ehrlich tumor, non-small cell lung, oat cell, papillary, bronchiolar, bronchogenic, squamous cell, and transitional cell), histiocytic disorders, leukemia (e.g., B cell, mixed cell, null cell, T cell, T-cell chronic, HTLV-II-associated, lymphocytic acute, lymphocytic chronic, mast cell, and myeloid), histiocytosis malignant, lymphoma (any kind, including T cell lymphoma, Hodgkin lymphoma, non-Hodgkin lymphoma), immunoproliferative small, plasmacytoma, reticuloendotheliosis, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing sarcoma, synovioma, adenofibroma, adenolymphoma, carcinosarcoma, chordoma, craniopharyngioma, dysgerminoma, hamartoma, mesenchymoma, mesonephroma, myosarcoma, ameloblastoma, cementoma, odontoma, teratoma, thymoma, trophoblastic tumor, adeno-carcinoma, adenoma, cholangioma, cholesteatoma, cylindroma, cystadenocarcinoma, cystadenoma, granulosa cell tumor, gynandroblastoma, hepatoma, hidradenoma, islet cell tumor, Leydig cell tumor, papilloma, Sertoli cell tumor, theca cell tumor, leiomyoma, leiomyosarcoma, myoblastoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma, phyllodes, fibrosarcoma, hemangiosarcoma, leimyosarcoma, leukosarcoma, liposarcoma, lymphangiosarcoma, myosarcoma, myxosarcoma, ovarian carcinoma, rhabdomyosarcoma, sarcoma (e.g., Ewing, experimental, Kaposi, and mast cell), neurofibromatosis, and cervical dysplasia, and other conditions in which cells have become immortalized or transformed.

“Cell death” as used herein refers to cell death by an accidental (necrosis) manner, which is a form of cell death that results from acute tissue injury and provokes an inflammatory response, cell death by a programmed pathway (programmed cell death) or cell death by autophagy.

“Programmed cell death (PCD)” as used herein means death of a cell in any form, mediated by an intracellular program. PCD is carried out in a regulated process which generally confers advantage during an organism's life-cycle. PCD serves fundamental functions during both plant and metazoa (multicellular animals) tissue development. Three types of PCD are characterized: (i) Apoptosis or Type I cell death; (ii) Autophagic or Type II cell death; (iii) “non-apoptotic programmed cell death” (or “caspase-independent programmed cell death” or “necrosis-like programmed cell death”) which is an alternative route to death are as efficient as apoptosis and can function as either backup mechanisms or the main type of PCD.

“Necrosis” as used herein means accidental death of cells and living tissue. Necrosis is less orderly than apoptosis. The disorderly death generally does not send cell signals which tell nearby phagocytes to engulf the dying cell. This lack of signaling makes it harder for the immune system to locate and recycle dead cells which have died through necrosis than if the cell had undergone cell death. The release of intracellular content after cellular membrane damage is the cause of inflammation in necrosis.

“Chemotherapy” as used herein means treatment of a subject with one or more pharmaceutical agents that kills cancer cells and/or slows the growth of cancer cells.

“Complement” or “complementary” as used herein refer to a nucleic acid may mean Watson-Crick (e.g., A-T/U and C-G) or Hoogsteen base pairing between nucleotides or nucleotide analogs of nucleic acid molecules. A full complement or fully complementary may mean 100% complementary base pairing between nucleotides or nucleotide analogs of nucleic acid molecules.

Hence, as used herein, complementarity refers to the capacity for precise pairing of two monomeric microRNA subunits regardless of where in miR or target miR the two are located. The microRNA and a target nucleic acid are “substantially complementary” to each other when a sufficient number of complementary positions in each molecule are occupied by monomeric subunits that can hydrogen bond with each other. Thus, the term “substantially complementary” is used to indicate a sufficient degree of precise pairing over a sufficient number of monomeric subunits such that stable and specific binding occurs between the miR and a target nucleic acid.

“Dose” as used herein means a specified quantity of a pharmaceutical agent provided in a single administration. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual.

“Dosage unit” as used herein means a form in which a pharmaceutical agent is provided. In certain embodiments, a dosage unit is a vial containing lyophilized oligonucleotide. In certain embodiments, a dosage unit is a vial containing reconstituted oligonucleotide.

“Duration” as used herein means the period of time during which an activity or event continues. In certain embodiments, the duration of treatment is the period of time during which doses of a pharmaceutical agent or pharmaceutical composition are administered.

“Identical” or “identity” as used herein in the context of two or more nucleic acids or polypeptide sequences may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST, BLAST 2.0, BLAT or Bowtie.

“Inhibit” as used herein may mean prevent, suppress, repress, reduce or eliminate.

“Label” as used herein may mean a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and other entities which can be made detectable. A label may be incorporated into nucleic acids and proteins at any position.

“Metastasis” as used herein means the process by which cancer spreads from the place at which it first arose as a primary tumor to other locations in the body. The metastatic progression of a primary tumor reflects multiple stages, including dissociation from neighboring primary tumor cells, survival in the circulation, and growth in a secondary location.

“Mismatch” as used herein means a nucleobase of a first nucleic acid that is not capable of pairing with a nucleobase at a corresponding position of a second nucleic acid.

“Modulation” as used herein refers to up regulation or down regulation of cell death or cell proliferation.

“Modified oligonucleotide” as used herein means an oligonucleotide having one or more modifications relative to a naturally occurring terminus, sugar, nucleobase, and/or internucleoside linkage. According to one embodiment, the modified oligonucleotide is a miRNA comprising a modification (e.g. labeled).

“Mutant” as used herein refers to a sequence in which at least a portion of the functionality of the sequence has been lost, for example, changes to the sequence in a promoter or enhancer region will affect at least partially the expression of a coding sequence in an organism. As used herein, the term “mutation,” refers to any change in a sequence in a nucleic acid sequence that may arise such as from a deletion, addition, substitution, or rearrangement. The mutation may also affect one or more steps that the sequence is involved in. For example, a change in a DNA sequence may lead to the synthesis of an altered mRNA and/or a protein that is active, partially active or inactive.

“Nucleic acid” or “oligonucleotide” or “polynucleotide” used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

A nucleic acid will generally contain phosphodiester bonds, although nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. No. 5,235,033 and U.S. Pat. No. 5,034,506, which are incorporated by reference. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino) propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH₂, NHR, NR2 or CN, wherein R is C1-C6 alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature 438:685-689 (2005), Soutschek et al., Nature 432:173-178 (2004), and US 2005/0107325, which are incorporated herein by reference. Additional modified nucleotides and nucleic acids are described in US 2005/0182005, which is incorporated herein by reference. Modifications of the ribose-phosphate backbone may be done for a variety of reasons, e.g., to increase the stability and half-life of such molecules in physiological environments, to enhance diffusion across cell membranes, or as probes on a biochip. The backbone modification may also enhance resistance to degradation, such as in the harsh endocytic environment of cells. The backbone modification may also reduce nucleic acid clearance by hepatocytes, such as in the liver and kidney. Mixtures of naturally occurring nucleic acids and analogs may be made; alternatively, mixtures of different nucleic acid analogs, and mixtures of naturally occurring nucleic acids and analogs may be made.

“Overall survival time” as used herein means the time period for which a subject survives after diagnosis of or treatment for a disease. In certain embodiments, the disease is cancer.

“Progression-free survival” means the time period for which a subject having a disease survives, without the disease getting worse. In certain embodiments, progression-free survival is assessed by staging or scoring the disease. In certain embodiments, progression-free survival of a subject having cancer is assessed by evaluating tumor size, tumor number, and/or metastasis.

“Reduced tumorigenicity” as used herein refers to the conversion of hyperproliferative (e.g., neoplastic) cells to a less proliferative state. In the case of tumor cells, “reduced tumorigenicity” is intended to mean tumor cells that have become less tumorigenic or non-tumorigenic or non-tumor cells whose ability to convert into tumor cells is reduced or eliminated. Cells with reduced tumorigenicity either form no tumors in vivo or have an extended lag time of weeks to months before the appearance of in vivo tumor growth. Cells with reduced tumorigenicity may also result in slower growing three dimensional tumor mass compared to the same type of cells having fully inactivated or non-functional tumor suppressor gene growing in the same physiological milieu (e.g., tissue, organism age, organism sex, time in menstrual cycle, etc.).

“Senescence” used herein may include permanent cessation of DNA replication and cell growth not reversible by growth factors, such as occurs at the end of the proliferative life span of normal cells or in normal or tumor cells in response to cytotoxic drugs, DNA damage or other cellular insult. Senescence is also characterized by certain morphological features, including increased size, flattened morphology increased granularity,

“Side effect” as used herein means a physiological response attributable to a treatment other than desired effects. In certain embodiments, side effects include, without limitation, injection site reactions, liver function test abnormalities, renal function abnormalities, liver toxicity, renal toxicity, central nervous system abnormalities, and myopathies. Such side effects may be detected directly or indirectly. For example, increased aminotransferase levels in serum may indicate liver toxicity or liver function abnormality. For example, increased bilirubin may indicate liver toxicity or liver function abnormality.

“Stringent hybridization conditions” used herein may mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5-10° C. lower than the thermal melting point (Tm) for the specific sequence at a defined ionic strength pH. The Tm may be the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may be those in which the salt concentration is less than about 1.0M sodium ion, such as about 0.01-1.0M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Substantially complementary” used herein may mean that a first sequence is at least 60%-99% identical to the complement of a second sequence over a region of 8-50 or more nucleotides, or that the two sequences hybridize under stringent hybridization conditions.

Substantially identical” used herein may mean that a first and second sequence are at least 60%-99% identical over a region of 8-50 or more nucleotides or amino acids, or with respect to nucleic acids, if the first sequence is substantially complementary to the complement of the second sequence.

“Subject” as used herein refers to a mammal, including both human and other mammals. In one particular embodiment the methods of the present invention are applied to human subjects.

“Therapeutically effective amount” or “therapeutically efficient” used herein as to a drug dosage, refer to dosage that provides the specific pharmacological response for which the drug is administered in a significant number of subjects in need of such treatment. The “therapeutically effective amount” may vary according, for example, the physical condition of the patient, the age of the patient and the severity of the disease.

“Therapy” as used herein means a disease treatment method. In certain embodiments, therapy includes, but is not limited to, chemotherapy, surgical resection, transplant, and/or chemoembolization.

“Treat” or “treating” used herein when referring to protection of a subject from a condition may mean preventing, suppressing, repressing, or eliminating the condition. Preventing the condition involves administering a composition described herein to a subject prior to onset of the condition. Suppressing the condition involves administering the composition to a subject after induction of the condition but before its clinical appearance. Repressing the condition involves administering the composition to a subject after clinical appearance of the condition such that the condition is reduced or prevented from worsening. Elimination of the condition involves administering the composition to a subject after clinical appearance of the condition such that the subject no longer suffers from the condition.

“Unit dosage form,” used herein may refer to a physically discrete unit suitable as a unitary dosage for a human or animal subject. Each unit may contain a predetermined quantity of a composition described herein, calculated in an amount sufficient to produce a desired effect in association with a pharmaceutically acceptable diluent, carrier or vehicle. The specifications for a unit dosage form may depend on the particular composition employed and the effect to be achieved, and the pharmacodynamics associated with the composition in the host.

“Variant” used herein to refer to a nucleic acid may mean (i) a portion of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto.

“Vector” used herein may mean a nucleic acid sequence containing an origin of replication. A vector may be a plasmid, bacteriophage, and bacterial artificial chromosome or yeast artificial chromosome. A vector may be a DNA or RNA vector. A vector may be either a self-replicating extrachromosomal vector or a vector which integrates into a host genome.

As used herein, the term “wild type” sequence refers to a coding, non-coding or interface sequence is an allelic form of sequence that performs the natural or normal function for that sequence. Wild type sequences include multiple allelic forms of a cognate sequence, for example, multiple alleles of a wild type sequence may encode silent or conservative changes to the protein sequence that a coding sequence encodes.

It is to be understood that the terminology used herein is for the purpose of describing particular embodiments only and it is not intended to be limiting. It must be noted that, as used in the specification and the appended claims, the singular forms “a,” “an” and “the” include plural referents unless the context clearly dictates otherwise.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

The following examples are presented in order to more fully illustrate some embodiments of the invention. They should, in no way be construed, however, as limiting the broad scope of the invention.

### EXAMPLES

### Materials and Methods

### 1. PG-NH₂ Synthesis

PG-NH₂ synthesis is described in detail in WO 2009/141170, which is incorporated herein in its entirety. Briefly, The PG-NH₂ compounds (amine-terminated polyglycerol compounds having one or more hydroxyls replaced by —NH₂ groups) are synthesized in a two-step protocol. In a first step, hyperbranched polyglycerol is reacted with mesylchloride in base to provide a mesylated PG. This is then reacted with sodium azide to yield PG bearing N₃ groups, which is then reduced with triphenylphosphine to yield PG-NH₂; such amines can be further further reacted, as is known in the art. Alternatively, the hyperbranched polyglyerol is activated to phenyl polyglycerol carbonate, followed by reaction with amines of different chain length to form amide-terminated polyglycerols. By this reaction pathway, it is possible to synthesize a library of different amine and amide derivatives based on a PG core.

### 2. PG-NH₂-miR Polyplex Generation

PG-NH₂-miR polyplexes are generated by gently mixing the PG-NH₂ nanocarrier with the microRNA in PBS for in vivo applications. For in vitro experiments the PG-NH₂ nanocarrier is mixed with the microRNA in DMEM medium without any additives. The polyplex-microRNA mixture is incubated for 30 minutes at room temperature and then added to cells, or injected to animals.

N/P ratio is one way to calculate the proportion of nanocarrier per nucleic acid in the polyplex. N/P stands for the ratio of amines (the nanocarrier moiety) per phosphate (the nucleic acid moiety).

The following calculation was used for conversion of molar to N/P ratio with the PG-NH₂ (90%) nanocarrier:

267 amines/mol nanopolymer
40 phosphate groups/mol of miRNA or siRNA
1:1 molar ratio=7 N/P ratio (267/40)
2:1 molar ratio=14 N/P ratio
5:1 molar ratio=35 N/P ratio

The following calculation was used for conversion of molar to N/P ratio with the FS-157 (PG90-FITC-SS-PEG 10%) nanocarrier:

86.8 amines/mol nanopolymer
40 phosphate groups/mol of miRNA or siRNA
1:1 molar ratio=2.2 N/P ratio (86.8/40)
2:1 molar ratio=4.4 N/P ratio
5:1 molar ratio=11 N/P ratio

### 3. General Methods of Molecular Biology

A number of methods of the molecular biology art are not detailed herein, as they are well known to the person of skill in the art. Such methods include PCR, expression of cDNAs, transfection of human cells, electrophoretic mobility shift assay (EMSA), and the like. Textbooks describing such methods are, e.g., Sambrook et al. (1989) Molecular Cloning, A Laboratory Manual, Cold Spring Harbor Laboratory, ISBN: 0879693096; F. M. Ausubel (1988) Current Protocols in Molecular Biology, ISBN: 047150338X, John Wiley & Sons, Inc. Methods of introducing nucleic acids, or nucleic acids comprised in delivery systems, into cells are well known in the art. Suitable methods include electroporation, particle gun technology, calcium phosphate precipitation, direct microinjection, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995. In some embodiments, lipofectamine and calcium mediated gene transfer technologies are used.

### 4. Cell Lines

U-87 MG (malignant glioma cell line) cells were obtained from the American Type Culture Collection (ATCC®; Manassas, Va., USA) and cultured in Dulbecco's modified Eagle's medium (DMEM) supplemented with 10% FBS, 100 U/ml penicillin, 100 U/ml streptomycin, 12.5 U/ml nystatin, and 2 mM L-glutamine (Biological Industries Ltd.)

A172 (human gliobastoma cell line) cells were obtained from ATCC® and cultured in DMEM supplemented with 10% FBS, 100 U/ml penicillin, 100 U/ml streptomycin, 12.5 U/ml nystatin, and 2 mM L-glutamine (Biological Industries Ltd.).

T88G (human gliobastoma cell line) cells were obtained from ATCC® and cultured in DMEM supplemented with 10% FBS, 100 U/ml penicillin, 100 U/ml streptomycin, 12.5 U/ml nystatin, and 2 mM L-glutamine (Biological Industries Ltd.).

Human umbilical vein endothelial cells (HUVEC) were obtained from (HUVECs; PCS-100-010) were purchased from ATCC® and cultured in EGM-2 medium (Lonza, Basel, Switzerland).

All cells were grown at 37° C. in 5% CO₂.

### 5. Glioblastoma Tumor Samples

Tumor resections (formalin-fixed paraffin embedded (FFPE) samples) from 12 short-term survival (STS) and 10 long-term survival (LTS) patients were profiled using microarrays. Survival times of the LTS patients were all more than 50 months. Survival times of the STS patients were less than 7 months. Survival times were calculated from the date of surgery up to the date of death, or up to the date of last contact with the patient. All patients were diagnosed with Glioblastoma (GBM), with the primary cancer location being the brain. In six (6) of the LTS patients, the sample was obtained at the time of the first surgery (also referred to as 1^{st} surgery LTS samples). In the other four (4) LTS patients, the sample was obtained from a subsequent surgery, which means that the patient might have undergone treatment prior to obtaining the sample. All the STS samples were obtained at the time of the first surgery. All of the LTS patients were treated with chemotherapy and radiation following surgery. Five (5) of the STS patients were treated with radiation following surgery and two of these were also treated with chemotherapy.

### 6. RNA Extraction

RNA extraction from FFPE samples was performed using an RNA-extraction kit (miRNeasy, Qiagen) according to the manufacturer's instructions.

### 7. MicroRNA Profiling

Custom microarrays (Agilent Technologies, Santa Clara, Calif.) were produced by printing DNA oligonucleotide probes to 2172 microRNA sequences, 17 negative controls, 22 spikes, and 10 positive controls (total of 2221 probes). Each microRNA probe, printed in triplicate, carried up to 28-nucleotide (nt) linker at the 3′ end of the microRNA complement sequence. Negative spikes and positive probes were printed from 3 to 200 times. Seventeen (17) negative control probes were designed using sequences which do not match the genome. Two groups of positive control probes were designed to hybridize with the microRNA array: (i) synthetic small RNAs, which were spiked to the RNA sample before labeling to verify labeling efficiency; and (ii) probes for abundant small RNA (e.g., small nuclear RNAs (U43, U24, Z30, U6, U48, U44)), 5.8s and 5s ribosomal RNA, which are spotted on the array to verify RNA quality.

### 8. RNA Cy-Dye Labelling

Five μg of total RNA were labeled by ligation (Thomson et al., Nature Methods 2004, 1:47-53) of an RNA-linker, p-rCrU-Cy/dye (Dharmacon), to the 3′ end with Cy3 or Cy5. The labeling reaction contained total RNA, spikes (0.1-20 fmoles), 300 ng RNA-linker-dye, 15% DMSO, 1× ligase buffer and 20 units of T4 RNA ligase (New England BioLabs®) and proceeded at 4° C. for 1 hour followed by 1 hour at 37° C. The labeled RNA was mixed with 3× hybridization buffer (Ambion), heated to 95° C. for 3 minutes and then added on top of the miRdicator™ array. Slides were hybridized 12-16 hours at 42° C., followed by two washes at room temperature with 1×SSC and 0.2% SDS and a final wash with 0.1×SSC.

Arrays were scanned using a microarray scanner (Microarray Scanner Bundle G2565BA, Agilent Technologies®) with a resolution of 5 m at XDR Hi 100%, XDR Lo 5%. Array images were analyzed using compatible software (Feature Extraction 10.7.1.1, Agilent®).

9. Statistical Analysis of the microRNA Profiling by Microarray

P-values were calculated using a two-sided (unpaired) Student's t-test on the log-transformed normalized fluorescence signal. The threshold for significant differences was determined by setting a p-value threshold to 0.05. For each differentially expressed microRNA, the fold-difference (ratio of the median normalized fluorescence) was calculated. Only miRs with a median signal above 300 in either group (for all comparisons) were tested.

### Example 1

### PG-NH₂-miR Polyplex Formation

The optimal ratio for the polyplex formation was studied by electrophoretic mobility shift assay (EMSA). 50 pmol of miRNA (miR34a and NC miR) was incubated with PG-NH₂ at 1:0.5, 1:1 and 1:2 molar ratios of miRNA to carrier, for 15 min at room temperature (RT). Mobility of free and nanocarrier-complexed miRNA at several N/P ratios was analyzed by agarose gel electrophoresis and is shown in FIG. 2. The best molar ratio was found to be 1:2.

### Example 2

### PG-NH₂-miR-34a Polyplex Inhibits Proliferation, Cell Cycle Progression, and Migration of Human Brain Cell Lines

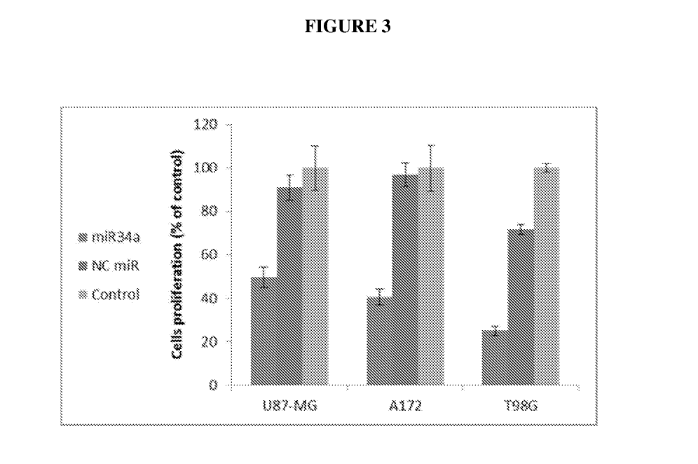

U-87 MG (malignant glioma cell line), A172 (human gliobastoma cell line) and T88G (human gliobastoma cell line) cells were plated onto a 24-well plate (5×10⁴ cells/well) in DMEM, supplemented with 10% FBS, and incubated for 24 h (37° C.; 5% CO2). The cells were then transfected with PG-NH₂-miR34a polyplex (100 nM-miR-equivalent). Cell viability was assessed by Coulter Counter following 7 days. FIG. 3 shows PG-NH₂-miR-34a polyplex inhibiting the growth of human glioblastoma cells in vitro. Comparative assays are made by transfecting the cells with miR34a.

### Example 3

### PG-NH₂-miR-34a Polyplex Inhibits Migration of Human Brain Cell Lines

U-87 MG and A172 human glioblastoma cells were transfected with hsa-miR-34a or NC-miR (100 nM-miR-equivalent) complexed with PG-NH₂. Three days later, the cell migration assay was performed using modified 8 mm Boyden chambers (two fluid-containing chambers separated by a microporous membrane). Following transfection, cells (2×10⁵ cells/200 μl) were added to the upper chamber of transwells and allowed to migrate towards the underside of the chamber for 6 hours in the presence of 10% fetal bovine serum (FBS)-containing media in the lower chamber. Untreated human umbilical vein endothelial cells (HUVEC) were seeded in a similar manner, and allowed to migrate towards conditioned media from U-87 MG and A172 cells following transfection. Cells were then fixed with ice-cold methanol and stained (Hema 3 Stain System). The stained migrated cells were imaged using an inverted microscope (Nikon TE2000E) integrated with Nikon DS5 cooled CCD camera by 10× objective, under bright field illumination. Migrated cells from the captured images per membrane were counted using NIH image software. Migration was normalized to percent migration, with 100% representing migration towards 10% FBS-containing media.

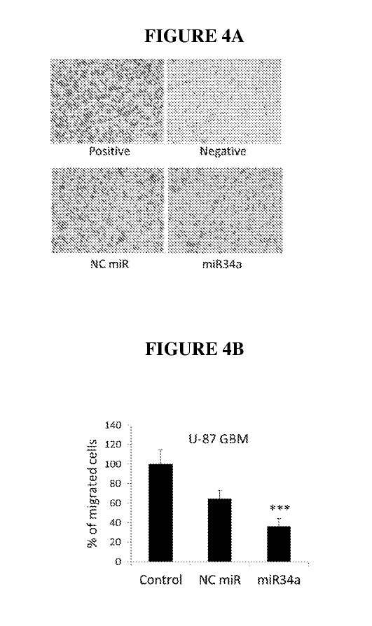

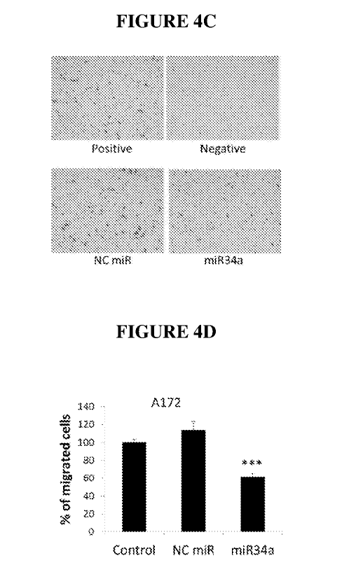

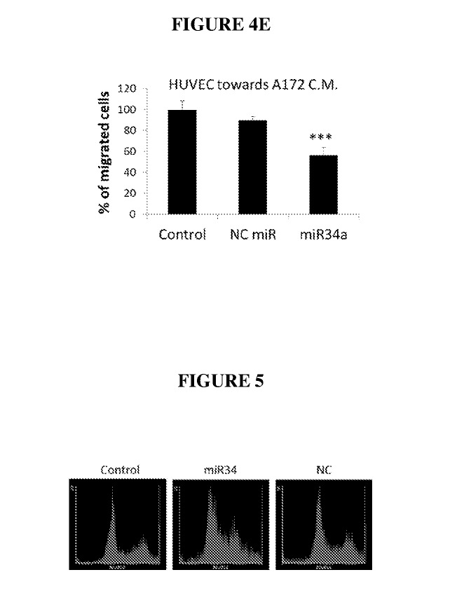

FIGS. 4A-4E show inhibition of brain cell migration upon PG-NH₂-miR34a polyplex treatment. As negative control, no FBS was added to the media in the lower chamber (FIGS. 4A and 4C, top right micrographs). Maximum migration is seen in non-transfected U87-MG and A172 cells in the presence of FBS cells (FIGS. 4A and 4C, top left micrographs). Transfection with PG-NH₂—NC induced slight inhibition of migration (FIGS. 4A and 4C, lower left micrographs, and FIGS. 4B and 4D). Transfection with PG-NH₂-miR-34a polyplex induced inhibition of migration at significant levels (FIGS. 4A and 4C, lower right micrographs, and FIGS. 4B and 4D). A similar inhibitory effect was observed on human umbilical vein endothelial cells (HUVEC), wherein HUVEC migration towards conditioned media (CM) from A172 cells transfected with the PG-NH₂-miR34a polyplex (FIG. 4E) was inhibited for about more than 40%. These experiments showed that hsa-miR-34a was capable of inhibiting cell migration when delivered through the nanocarrier-microRNA complex described herein.

### Example 4

### PG-NH₂-miR-34a Polyplex Induces Cell Cycle Arrest in Human Brain Cell Lines

U-87 MG cells were transfected with PG-NH₂-hsa-miR-34a or PG-NH₂—NC (100 nM-miR-equivalent). Cells were harvested 72 hours later, fixed, stained with propidium iodide and analyzed by flow cytometry. FIG. 5 shows the induction of S-phase arrest in U-87 MG cells by miR-34a overexpression. Table 3 presents the number of cells detected by flow cytometry at each cell cycle phase, G0/G1, S- or G2/M in untreated cells (control), cells treated with miR negative control (PG-NH₂—NC, SEQ ID NO: 147), and cells treated with PG-NH₂-miR-34a (SEQ ID NO:63). It may be noted that the number of cells in S-phase increased while the number of cells in G2/M decreased following PG-NH₂-miR34a treatment, indicating that transfection with hsa-miR-34a induced cell-cycle arrest.

TABLE 3
Number of cells at G0/G1, S- or G2/M in control, PG-
NH₂-miR-34a-treated and PG-NH₂-NC-treated cells

| Cell Cycle Phase | Control | PG-NH₂-miR-34a | PG-NH₂-NC |
|---|---|---|---|
| G0/G1 | 38.84 | 42.46 | 40.72 |
| S-phase | 12.99 | 23.55 | 15.35 |
| G2/M | 26.3 | 11.17 | 22.5 |

### Example 5

### PG-NH₂-miR-34a Polyplex Inhibits the Expression of miRNA Targets C-Met and Notch in Human Brain Cell Lines

The inventors further studied the expression targets and functional effects of hsa-miR-34a in human glioblastoma. Transfection of miR-34a using the novel nanocarrier down-regulated hsa-miR-34a validated targets in several human glioblastoma cell lines.

hsa-miR-34a (100 nM) was complexed with PG-NH₂ nanocarrier (500 nM) in serum-free medium, incubated for 20 minutes at room temperature, and then added to U-87 MG cells. RNA was isolated 48 hours later, and qPCR was performed for hsa-miR-34a and C-Met expression levels, which were normalized to TBP and RPS20 (housekeeping genes). Protein extracts were analyzed by SDS-PAGE followed by Western blot using anti-C-Met, anti-Notch1 or anti-beta actin antibodies (loading control).

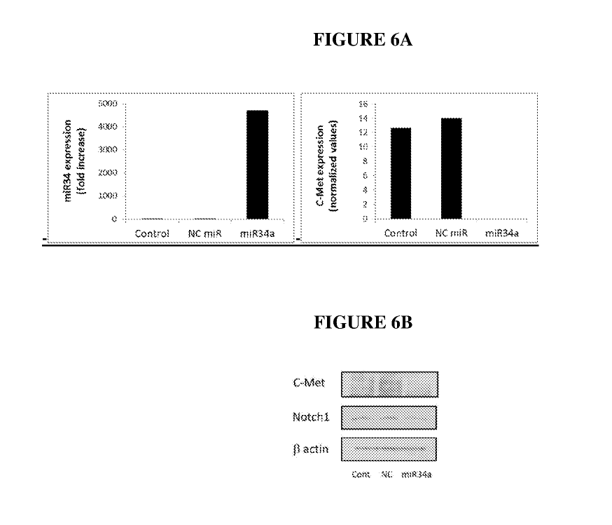

U-87 MG cells transfected with PG-NH₂-miR-34a polyplex exhibited a ˜5000-fold increase in the expression of hsa-miR-34a (FIG. 6A, left-hand histogram) and a major decrease in the expression of its target genes c-Met (FIG. 6A, right-hand histogram) and Notch1 (FIG. 6B, showing a Western blot of protein extracts from U-87 MG cells untransfected (Cont lane), transfected with NC miR (NC lane) or transfected with hsa-miR-34a (miR-34a lane). Thus, PG-NH₂-miR-34a treatment induced inhibition/down-regulation of c-Met expression, as well as inhibition/down-regulation of Notch1 expression.

### Example 6

### In Vivo Therapeutic Effect of the PG-NH₂-miR-34a Polyplex in a Glioblastoma Mouse Model

The ability of the PG-NH₂-miR-34a polyplex to inhibit tumor growth and survival in a U87-cell glioblastoma tumor model in SCID mice was evaluated. mCherry-labeled U87 MG human glioblastoma cells were subcutaneously inoculated in the flank of SCID mice (1×10⁶ cells in 100 μl PBS). Treatment started approximately 4 weeks after U87 MG glioblastoma cell injection, when tumors reached the average volume of 50 mm³. Mice received three (3) consecutive treatments (on day 0, 3 and 6) of PG-NH₂-miR-34a (10 mg/kg PG-NH₂, 4 mg/kg miR-34a), PG-NH₂—NC-miR (10 mg/kg PG-NH₂, 4 mg/kg negative control miR) or saline. All treatments were administered intra-tumor in a total volume of 20 μl.

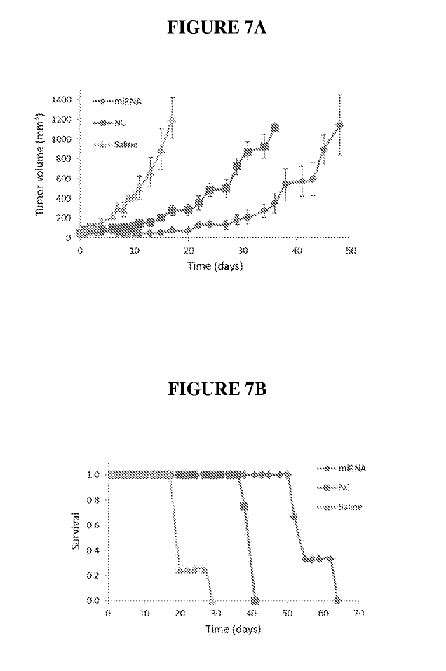

The results are shown in FIGS. 7A-7B. While saline treated mice lived for 30 days and mice treated with PG-NH₂—NC survived for 40 days, mice that were administered with PG-NH₂-miR-34a survived for 62 days. Data in tumor volume graph represents mean±s.e.m. (for PG-NH₂-miR-34a treated compared with PG-NH₂—NC treated mice, p<0.05 on days 20 to 30, p<0.01 on days 32 to 62).

These results clearly demonstrate that the PG-NH₂-miR-34a polyplex significantly inhibited glioblastoma tumor growth, in contrast to the PG-NH₂—NC polyplex which elicited only a slight inhibition in tumor growth. Mice survival was also considerably improved by the PG-NH₂-miR-34a polyplex treatment. While saline-treated mice survived for 30 days, PG-NH₂—NC-treated mice lived for 40 days, and the PG-NH₂-miR-34a-treated group survived for 62 days. Thus, hsa-miR-34a induced inhibition of tumor growth and increase in survival.

### Example 7

### Analysis of microRNA Expression in Samples from Glioblastoma Patients

In order to identify potential therapeutic microRNA to be conjugated or complexed with the PG-NH₂ polyplex system described herein, three analysis of miR expression were performed in samples from long-term survivor (LTS) or short-term survivor (STS) gliobastoma human patients, as detailed below.

I—Specific microRNAs are Differentially Expressed Between LTS and STS Samples

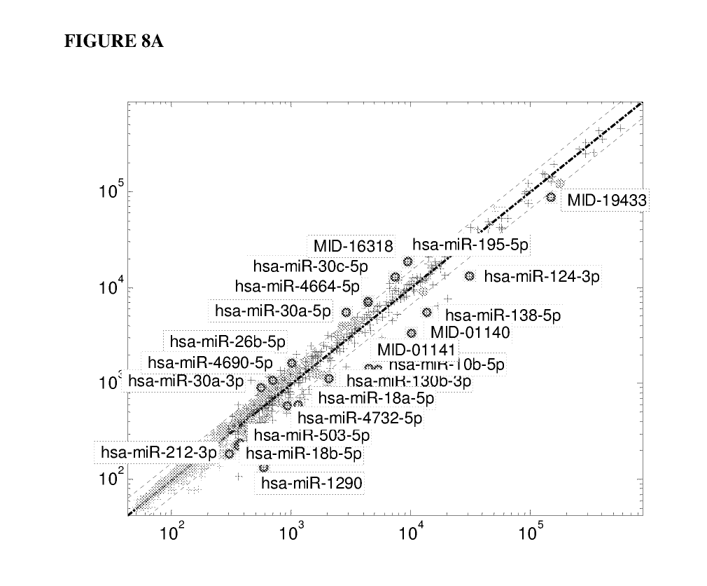

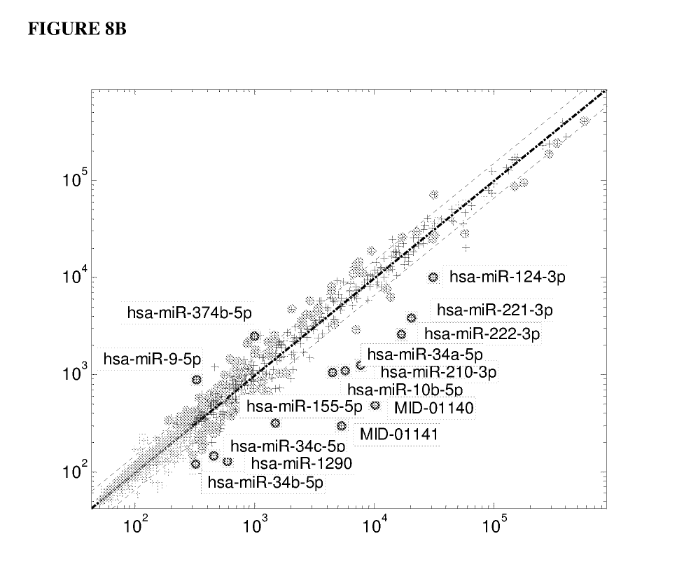

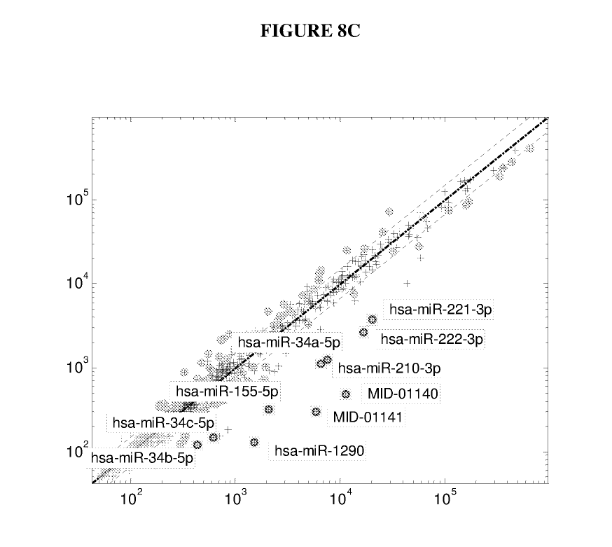

MicroRNA expression levels in samples of LTS (n=10) and STS (n=12) tumors were compared. The results exhibited a set of 55 miRs that was differentially expressed (p-value<0.05), as shown in Tables 4-5 and FIG. 8A. These results provide the potential therapeutic miRs to be delivered with the nanocarrier system described herein. Upregulated miRs in LTS samples may be candidates for mimetic or anti-miR therapy. Similarly, upregulated miRs in the STS samples represent miRNAs that may be candidates for mimetic or anti-miR therapy.

TABLE 4
Upregulated in LTS vs STS

| | | | LTS | STS |
|---|---|---|---|---|
| | p- | fold- | median | median |
| microRNA | value^{a} | change^{b} | value^{c} | value^{c} |
| MID-16318 | 1.50E−02 | 1.97 | 1.90E+04 | 9.50E+03 |
| hsa-miR-30a-5p | 1.60E−04 | 1.91 | 5.50E+03 | 2.90E+03 |
| hsa-miR-195-5p | 4.40E−02 | 1.73 | 1.30E+04 | 7.50E+03 |
| hsa-miR-4664-5p | 2.40E−02 | 1.63 | 7.20E+03 | 4.40E+03 |
| hsa-miR-30a-3p | 2.80E−04 | 1.6 | 9.00E+02 | 5.60E+02 |
| hsa-miR-26b-5p | 1.00E−03 | 1.6 | 1.60E+03 | 1.00E+03 |
| hsa-miR-30c-5p | 1.00E−03 | 1.58 | 6.90E+03 | 4.40E+03 |
| hsa-miR-4690-5p | 4.90E−02 | 1.55 | 1.10E+03 | 7.00E+02 |
| MID-23291 | 1.50E−02 | 1.48 | 4.00E+03 | 2.70E+03 |
| hsa-miR-29a-3p | 2.00E−02 | 1.4 | 2.40E+04 | 1.70E+04 |
| hsa-miR-30d-5p | 1.80E−03 | 1.34 | 4.00E+03 | 3.00E+03 |
| hsa-miR-152-3p | 2.40E−02 | 1.33 | 1.40E+03 | 1.10E+03 |
| hsa-miR-1273f | 4.00E−02 | 1.32 | 7.70E+02 | 5.80E+02 |
| hsa-miR-30e-3p | 2.40E−04 | 1.32 | 3.50E+02 | 2.70E+02 |
| hsa-miR-5001-5p | 3.20E−02 | 1.31 | 1.70E+03 | 1.30E+03 |
| hsa-miR-1285-3p | 4.80E−02 | 1.3 | 5.50E+02 | 4.20E+02 |
| hsa-miR-30b-5p | 9.10E−03 | 1.3 | 4.40E+03 | 3.40E+03 |
| hsa-miR-361-5p | 5.60E−03 | 1.3 | 3.20E+03 | 2.50E+03 |
| hsa-miR-3653 | 4.70E−02 | 1.29 | 4.70E+02 | 3.70E+02 |
| hsa-miR-30e-5p | 2.90E−03 | 1.27 | 1.30E+03 | 9.90E+02 |
| hsa-miR-374c-5p | 5.50E−03 | 1.27 | 1.10E+03 | 8.60E+02 |
| hsa-miR-4800-3p | 3.20E−02 | 1.26 | 3.30E+02 | 2.60E+02 |
| hsa-miR-4656 | 3.40E−02 | 1.26 | 8.50E+02 | 6.70E+02 |
| hsa-miR-30c-2-3p | 1.30E−03 | 1.25 | 3.30E+02 | 2.60E+02 |
| hsa-miR-5684 | 4.30E−02 | 1.24 | 6.10E+02 | 4.90E+02 |
| hsa-miR-374b-5p | 7.40E−03 | 1.24 | 1.20E+03 | 9.90E+02 |
| hsa-miR-505-5p | 1.30E−02 | 1.23 | 4.80E+02 | 3.90E+02 |
| MID-00206 | 1.70E−02 | 1.21 | 6.80E+02 | 5.60E+02 |
| MID-00745 | 3.40E−02 | 1.2 | 8.90E+02 | 7.40E+02 |
| hsa-let-7f-5p | 4.50E−02 | 1.14 | 2.60E+04 | 2.30E+04 |
| hsa-miR-4640-5p | 4.20E−02 | 1.1 | 3.60E+02 | 3.30E+02 |

^{a}p-values were calculated using a two-sided (unpaired) Student's t-test.
^{b}Fold-change represents the ratio between the median values of each group.
^{c}Median values: median of expression values (rounded).

TABLE 5
MicroRNAs up-regulated in STS vs LTS

| | | | LTS | STS |
|---|---|---|---|---|
| | p- | fold- | median | median |
| microRNA | value^{a} | change^{b} | value^{c} | value^{c} |
| hsa-miR-1290 | 2.20E−02 | 4.45 | 1.30E+02 | 5.90E+02 |
| MID-01141 | 6.20E−03 | 3.83 | 1.40E+03 | 5.30E+03 |
| hsa-miR-10b-5p | 8.10E−03 | 3.1 | 1.40E+03 | 4.40E+03 |
| MID-01140 | 2.30E−02 | 3.03 | 3.30E+03 | 1.00E+04 |
| hsa-miR-138-5p | 1.40E−02 | 2.47 | 5.60E+03 | 1.40E+04 |
| hsa-miR-124-3p | 4.50E−02 | 2.35 | 1.30E+04 | 3.10E+04 |
| hsa-miR-4732-5p | 4.30E−02 | 1.91 | 5.90E+02 | 1.10E+03 |
| hsa-miR-130b-3p | 4.50E−02 | 1.84 | 1.10E+03 | 2.10E+03 |
| MID-19433 | 4.60E−02 | 1.7 | 8.80E+04 | 1.50E+05 |
| hsa-miR-212-3p | 7.90E−03 | 1.64 | 1.80E+02 | 3.00E+02 |
| hsa-miR-503-5p | 1.20E−02 | 1.63 | 2.20E+02 | 3.60E+02 |
| hsa-miR-18a-5p | 9.80E−03 | 1.59 | 5.80E+02 | 9.20E+02 |
| hsa-miR-18b-5p | 1.90E−02 | 1.54 | 2.40E+02 | 3.70E+02 |
| hsa-miR-3200-3p | 2.60E−02 | 1.49 | 2.30E+02 | 3.40E+02 |
| hsa-miR-132-3p | 4.00E−02 | 1.48 | 1.20E+03 | 1.80E+03 |
| MID-14865 | 1.90E−03 | 1.45 | 1.20E+05 | 1.80E+05 |
| MID-15867 | 1.00E−02 | 1.4 | 9.00E+03 | 1.30E+04 |
| hsa-miR-378c | 3.60E−02 | 1.35 | 4.60E+02 | 6.20E+02 |
| hsa-miR-378g | 3.90E−02 | 1.31 | 3.20E+02 | 4.20E+02 |
| hsa-miR-339-5p | 3.30E−02 | 1.3 | 3.50E+02 | 4.50E+02 |
| hsa-miR-769-5p | 3.10E−02 | 1.29 | 3.10E+02 | 4.00E+02 |
| hsa-miR-425-5p | 3.50E−03 | 1.29 | 1.30E+03 | 1.60E+03 |
| hsa-miR-378a-3p | 4.80E−02 | 1.24 | 6.40E+02 | 8.00E+02 |
| hsa-miR-542-5p | 5.00E−02 | 1.16 | 4.00E+02 | 4.70E+02 |

^{a}p-values were calculated using a two-sided (unpaired) Student's t-test.
^{b}Fold-change represents the ratio between the median values of each group.
^{c}Median values: median of expression values (rounded).

II—Specific microRNAs are Differentially Expressed Between LTS Samples Obtained from the 1^{st} Surgery and STS Samples

In order to compare only the primary tumors from the STS and LTS samples, microRNA expression levels in samples from LTS tumors obtained at the first surgery (n=6) and STS (n=12) tumors were compared. The results exhibited a set of 108 miRs that was differentially expressed (p-value <0.05), as shown in Tables 6-7 and FIG. 8B. Of these 108 differentially expressed miRs, 48 were also differentially expressed when comparing all LTS and STS samples.

TABLE 6
MicroRNAs up-regulated in 1^{st} surgery LTS versus STS

| | | | LTS | STS |
|---|---|---|---|---|
| | p- | fold- | median | median |
| microRNA | value^{a} | change^{b} | value^{c} | value^{c} |
| hsa-miR-9-5p | 1.30E−02 | 2.7 | 8.80E+02 | 3.30E+02 |
| hsa-miR-374b-5p | 1.30E−04 | 2.53 | 2.50E+03 | 9.90E+02 |
| hsa-miR-99a-3p | 9.00E−03 | 2.5 | 2.20E+03 | 8.90E+02 |
| hsa-miR-374c-5p | 9.20E−05 | 2.44 | 2.10E+03 | 8.60E+02 |
| hsa-miR-181a-2-3p | 4.30E−02 | 2.34 | 1.50E+03 | 6.20E+02 |
| hsa-miR-708-5p | 1.60E−03 | 2.3 | 4.70E+03 | 2.00E+03 |
| hsa-miR-99a-5p | 2.10E−03 | 2.24 | 7.10E+04 | 3.20E+04 |
| MID-16318 | 4.20E−03 | 1.99 | 1.90E+04 | 9.50E+03 |
| hsa-miR-30a-5p | 1.40E−03 | 1.96 | 5.70E+03 | 2.90E+03 |
| hsa-miR-195-5p | 1.70E−02 | 1.92 | 1.40E+04 | 7.50E+03 |
| hsa-miR-4324 | 2.00E−02 | 1.91 | 1.30E+04 | 7.00E+03 |
| hsa-miR-125b-2-3p | 7.20E−03 | 1.89 | 1.40E+03 | 7.30E+02 |
| hsa-miR-4664-5p | 1.00E−02 | 1.83 | 8.00E+03 | 4.40E+03 |
| hsa-miR-708-3p | 1.20E−03 | 1.8 | 3.40E+02 | 1.90E+02 |
| hsa-miR-26b-5p | 1.30E−03 | 1.79 | 1.80E+03 | 1.00E+03 |
| hsa-miR-30a-3p | 2.30E−03 | 1.76 | 9.90E+02 | 5.60E+02 |
| MID-18468 | 1.80E−02 | 1.74 | 2.50E+03 | 1.50E+03 |
| hsa-miR-1180-3p | 7.90E−03 | 1.7 | 5.80E+02 | 3.40E+02 |
| hsa-miR-652-3p | 1.10E−02 | 1.69 | 2.20E+03 | 1.30E+03 |
| hsa-miR-30e-5p | 7.70E−04 | 1.68 | 1.70E+03 | 9.90E+02 |
| hsa-miR-30c-5p | 4.20E−03 | 1.67 | 7.30E+03 | 4.40E+03 |
| hsa-miR-497-5p | 1.10E−02 | 1.64 | 1.10E+04 | 6.70E+03 |
| hsa-miR-30e-3p | 4.10E−04 | 1.59 | 4.20E+02 | 2.70E+02 |
| hsa-miR-4690-5p | 4.70E−02 | 1.58 | 1.10E+03 | 7.00E+02 |
| hsa-miR-374a-5p | 7.80E−03 | 1.57 | 3.70E+02 | 2.40E+02 |
| MID-23291 | 1.70E−03 | 1.56 | 4.20E+03 | 2.70E+03 |
| hsa-miR-345-5p | 3.70E−02 | 1.56 | 2.10E+03 | 1.40E+03 |
| hsa-miR-29a-3p | 3.70E−02 | 1.52 | 2.60E+04 | 1.70E+04 |
| hsa-miR-5010-5p | 5.00E−02 | 1.51 | 8.00E+02 | 5.30E+02 |
| hsa-miR-5684 | 2.10E−02 | 1.5 | 7.40E+02 | 4.90E+02 |
| MID-17144 | 3.60E−02 | 1.49 | 1.10E+04 | 7.60E+03 |
| hsa-miR-421 | 3.10E−02 | 1.47 | 6.30E+02 | 4.30E+02 |
| hsa-miR-361-5p | 3.50E−06 | 1.46 | 3.60E+03 | 2.50E+03 |
| hsa-miR-30b-5p | 1.80E−03 | 1.45 | 4.90E+03 | 3.40E+03 |
| MID-18336 | 2.70E−02 | 1.42 | 1.10E+04 | 8.00E+03 |
| hsa-miR-504-5p | 1.40E−05 | 1.38 | 3.10E+02 | 2.20E+02 |
| hsa-miR-1271-5p | 3.20E−02 | 1.38 | 5.00E+02 | 3.60E+02 |
| hsa-miR-185-3p | 3.40E−02 | 1.38 | 3.90E+02 | 2.90E+02 |
| hsa-miR-505-5p | 1.70E−03 | 1.38 | 5.40E+02 | 3.90E+02 |
| hsa-miR-4800-3p | 7.50E−03 | 1.36 | 3.60E+02 | 2.60E+02 |
| hsa-miR-30d-5p | 7.00E−04 | 1.36 | 4.10E+03 | 3.00E+03 |
| hsa-miR-423-5p | 5.80E−04 | 1.35 | 4.60E+03 | 3.40E+03 |
| hsa-miR-6511a-5p | 2.70E−02 | 1.34 | 4.20E+02 | 3.10E+02 |
| hsa-miR-3907 | 1.30E−02 | 1.34 | 3.60E+02 | 2.70E+02 |
| hsa-miR-5001-5p | 3.60E−02 | 1.33 | 1.70E+03 | 1.30E+03 |
| hsa-miR-744-5p | 2.90E−02 | 1.33 | 3.30E+03 | 2.50E+03 |
| hsa-miR-3185 | 4.70E−02 | 1.31 | 1.10E+03 | 8.70E+02 |
| hsa-miR-30c-2-3p | 4.50E−03 | 1.31 | 3.40E+02 | 2.60E+02 |
| MID-00745 | 3.80E−02 | 1.31 | 9.70E+02 | 7.40E+02 |
| hsa-let-7f-5p | 4.80E−02 | 1.3 | 3.00E+04 | 2.30E+04 |
| hsa-miR-3653 | 2.40E−02 | 1.29 | 4.70E+02 | 3.70E+02 |
| MID-00206 | 2.40E−02 | 1.27 | 7.10E+02 | 5.60E+02 |
| MID-00612 | 2.70E−02 | 1.26 | 3.60E+02 | 2.80E+02 |
| hsa-miR-324-3p | 3.40E−02 | 1.18 | 1.60E+03 | 1.30E+03 |
| hsa-miR-4695-5p | 4.70E−02 | 1.12 | 5.90E+02 | 5.30E+02 |

^{a}p-values were calculated using a two-sided (unpaired) Student's t-test.
^{b}Fold-change represents the ratio between the median values of each group.
^{c}Median values: median of expression values (rounded).

TABLE 7
MicroRNAs up-regulated in STS vs 1st surgery LTS

| | | | LTS | STS |
|---|---|---|---|---|
| | p- | fold- | median | median |
| microRNA | value^{a} | change^{b} | value^{c} | value^{c} |
| MID-01140 | 1.50E−04 | 20.96 | 4.80E+02 | 1.00E+04 |
| MID-01141 | 7.20E−05 | 17.85 | 3.00E+02 | 5.30E+03 |
| hsa-miR-222-3p | 1.20E−02 | 6.42 | 2.60E+03 | 1.70E+04 |
| hsa-miR-34a-5p | 1.80E−02 | 6.13 | 1.20E+03 | 7.60E+03 |
| hsa-miR-221-3p | 1.20E−02 | 5.4 | 3.80E+03 | 2.00E+04 |
| hsa-miR-210-3p | 1.70E−02 | 5.12 | 1.10E+03 | 5.70E+03 |
| hsa-miR-155-5p | 1.10E−02 | 4.67 | 3.20E+02 | 1.50E+03 |
| hsa-miR-1290 | 2.60E−02 | 4.55 | 1.30E+02 | 5.90E+02 |
| hsa-miR-10b-5p | 1.00E−02 | 4.23 | 1.10E+03 | 4.40E+03 |
| hsa-miR-124-3p | 4.70E−02 | 3.09 | 1.00E+04 | 3.10E+04 |
| hsa-miR-34c-5p | 8.60E−03 | 3.09 | 1.50E+02 | 4.50E+02 |
| hsa-miR-34b-5p | 1.10E−02 | 2.65 | 1.20E+02 | 3.20E+02 |
| hsa-miR-15b-5p | 7.00E−03 | 2.46 | 2.90E+03 | 7.10E+03 |
| hsa-miR-138-5p | 3.90E−02 | 2.21 | 6.20E+03 | 1.40E+04 |
| hsa-miR-148a-3p | 3.20E−02 | 2.1 | 1.70E+02 | 3.60E+02 |
| hsa-miR-451a | 4.60E−02 | 2.04 | 2.80E+04 | 5.70E+04 |
| hsa-miR-212-3p | 1.60E−02 | 2.04 | 1.50E+02 | 3.00E+02 |
| hsa-miR-18a-5p | 1.80E−02 | 1.93 | 4.80E+02 | 9.20E+02 |
| hsa-miR-18b-5p | 1.10E−02 | 1.92 | 1.90E+02 | 3.70E+02 |
| hsa-miR-424-5p | 3.00E−02 | 1.88 | 6.70E+02 | 1.30E+03 |
| MID-14865 | 3.20E−04 | 1.88 | 9.50E+04 | 1.80E+05 |
| hsa-miR-503-5p | 1.40E−03 | 1.84 | 2.00E+02 | 3.60E+02 |
| hsa-miR-339-5p | 6.90E−03 | 1.81 | 2.50E+02 | 4.50E+02 |
| hsa-miR-132-3p | 4.00E−02 | 1.78 | 1.00E+03 | 1.80E+03 |
| MID-19433 | 2.30E−02 | 1.74 | 8.60E+04 | 1.50E+05 |
| hsa-miR-671-5p | 1.00E−02 | 1.69 | 3.70E+02 | 6.30E+02 |
| hsa-miR-199a-3p | 4.40E−02 | 1.68 | 7.00E+02 | 1.20E+03 |
| hsa-miR-542-5p | 3.60E−02 | 1.68 | 2.80E+02 | 4.70E+02 |
| MID-15867 | 6.30E−03 | 1.67 | 7.60E+03 | 1.30E+04 |
| MID-19149 | 4.40E−02 | 1.64 | 5.80E+02 | 9.50E+02 |
| hsa-miR-425-5p | 1.60E−03 | 1.58 | 1.00E+03 | 1.60E+03 |
| hsa-miR-4521 | 3.50E−02 | 1.57 | 2.30E+02 | 3.60E+02 |
| hsa-miR-378c | 2.20E−02 | 1.57 | 3.90E+02 | 6.20E+02 |
| hsa-miR-5100 | 1.20E−02 | 1.56 | 1.90E+05 | 2.90E+05 |
| hsa-miR-129-1-3p | 2.80E−02 | 1.56 | 4.00E+02 | 6.20E+02 |
| hsa-miR-1229-5p | 3.80E−02 | 1.56 | 4.40E+02 | 6.80E+02 |
| hsa-miR-214-3p | 3.10E−02 | 1.53 | 3.40E+02 | 5.10E+02 |
| hsa-miR-378g | 2.30E−02 | 1.52 | 2.80E+02 | 4.20E+02 |
| hsa-miR-3200-3p | 1.00E−02 | 1.49 | 2.30E+02 | 3.40E+02 |
| hsa-miR-625-5p | 3.90E−02 | 1.45 | 3.00E+02 | 4.40E+02 |
| MID-24702 | 3.40E−02 | 1.43 | 2.40E+05 | 3.40E+05 |
| hsa-miR-185-5p | 4.00E−02 | 1.43 | 3.30E+03 | 4.60E+03 |
| hsa-miR-4454 | 3.10E−02 | 1.42 | 4.10E+05 | 5.80E+05 |
| hsa-miR-422a | 2.50E−02 | 1.42 | 2.60E+02 | 3.70E+02 |
| hsa-miR-769-5p | 8.80E−03 | 1.38 | 2.90E+02 | 4.00E+02 |
| hsa-miR-4306 | 2.40E−02 | 1.37 | 7.20E+02 | 9.80E+02 |
| hsa-miR-378d | 4.60E−02 | 1.36 | 2.90E+02 | 3.90E+02 |
| hsa-miR-378a-3p | 4.00E−02 | 1.36 | 5.90E+02 | 8.00E+02 |
| hsa-miR-378i | 4.70E−02 | 1.34 | 3.00E+02 | 4.10E+02 |
| hsa-miR-199a-5p | 3.40E−02 | 1.29 | 8.70E+02 | 1.10E+03 |
| MID-00689 | 4.10E−02 | 1.27 | 4.90E+02 | 6.30E+02 |
| hsa-miR-107 | 1.40E−02 | 1.24 | 2.50E+04 | 3.00E+04 |
| hsa-miR-103a-3p | 3.20E−02 | 1.18 | 2.70E+04 | 3.20E+04 |

^{a}p-values were calculated using a two-sided (unpaired) Student's t-test.
^{b}Fold-change represents the ratio between the median values of each group.
^{c}Median values: median of expression values (rounded).

III—Specific microRNAs are Differentially Expressed Between 1^{st} Surgery LTS Samples and STS Samples for Patients Who Underwent Both Radiation and Chemotherapy or Who Had Extremely Low Survival Times (Under 60 Days)

In order to ensure that the differences in survival time were not due to treatment, microRNA expression levels were compared between samples from 1^{st} surgery LTS patients and STS patients who were either treated or had extremely low survival times (under 60 days). The results exhibited a set of 107 miRs that was differentially expressed (p-value <0.05), as shown in Tables 8-9 and FIG. 8C. From these 107 differentially expressed miRs, 36 were also differentially expressed when comparing all LTS and STS patients, and 76 were also differentially expressed when comparing 1^{st} surgery LTS and STS patients.

TABLE 8
MicroRNAs up-regulated in 1^{st} surgery LTS versus STS patients that
were treated or who had extremely short survival times (<60 days)

| | | | LTS | STS |
|---|---|---|---|---|
| | p- | fold- | median | median |
| microRNA | value^{a} | change^{b} | value^{c} | value^{c} |
| hsa-miR-374c-5p | 3.80E−04 | 2.88 | 2.10E+03 | 7.30E+02 |
| hsa-miR-99a-3p | 8.80E−03 | 2.86 | 2.20E+03 | 7.70E+02 |
| hsa-miR-374b-5p | 5.50E−04 | 2.84 | 2.50E+03 | 8.80E+02 |
| hsa-miR-9-5p | 2.00E−02 | 2.7 | 8.80E+02 | 3.30E+02 |
| hsa-miR-181a-2-3p | 2.60E−02 | 2.65 | 1.50E+03 | 5.50E+02 |
| hsa-miR-301a-3p | 3.70E−02 | 2.41 | 1.10E+03 | 4.80E+02 |
| hsa-miR-99a-5p | 3.80E−03 | 2.41 | 7.10E+04 | 2.90E+04 |
| hsa-miR-708-5p | 2.60E−03 | 2.3 | 4.70E+03 | 2.00E+03 |
| hsa-miR-195-5p | 8.30E−04 | 2.18 | 1.40E+04 | 6.60E+03 |
| hsa-miR-9-3p | 3.60E−02 | 2.17 | 2.50E+04 | 1.20E+04 |
| hsa-miR-4324 | 8.40E−04 | 2.09 | 1.30E+04 | 6.40E+03 |
| hsa-miR-652-3p | 1.30E−02 | 2.05 | 2.20E+03 | 1.10E+03 |
| hsa-miR-30a-5p | 3.80E−04 | 1.96 | 5.70E+03 | 2.90E+03 |
| hsa-miR-125b-2-3p | 7.00E−03 | 1.96 | 1.40E+03 | 7.00E+02 |
| hsa-miR-1180-3p | 1.10E−02 | 1.9 | 5.80E+02 | 3.10E+02 |
| hsa-miR-629-5p | 2.00E−02 | 1.89 | 3.60E+02 | 1.90E+02 |
| hsa-miR-454-3p | 1.80E−03 | 1.87 | 5.50E+02 | 2.90E+02 |
| MID-17144 | 4.80E−02 | 1.81 | 1.10E+04 | 6.20E+03 |
| hsa-miR-708-3p | 3.80E−03 | 1.8 | 3.40E+02 | 1.90E+02 |
| hsa-miR-26b-5p | 1.00E−03 | 1.78 | 1.80E+03 | 1.00E+03 |
| hsa-miR-497-5p | 2.70E−03 | 1.78 | 1.10E+04 | 6.20E+03 |
| hsa-miR-374a-5p | 3.40E−03 | 1.76 | 3.70E+02 | 2.10E+02 |
| hsa-miR-30a-3p | 3.20E−04 | 1.76 | 9.90E+02 | 5.60E+02 |
| hsa-miR-30e-5p | 4.00E−03 | 1.69 | 1.70E+03 | 9.80E+02 |
| hsa-miR-5684 | 3.00E−02 | 1.69 | 7.40E+02 | 4.40E+02 |
| hsa-miR-30c-5p | 7.50E−03 | 1.66 | 7.30E+03 | 4.40E+03 |
| hsa-miR-181c-3p | 3.70E−02 | 1.63 | 9.60E+02 | 5.90E+02 |
| hsa-miR-3607-3p | 2.00E−02 | 1.62 | 5.40E+02 | 3.40E+02 |
| hsa-miR-30e-3p | 5.00E−04 | 1.59 | 4.20E+02 | 2.70E+02 |
| hsa-miR-100-5p | 7.00E−04 | 1.58 | 4.10E+04 | 2.60E+04 |
| hsa-miR-345-5p | 2.20E−02 | 1.56 | 2.10E+03 | 1.40E+03 |
| hsa-miR-130a-3p | 3.90E−02 | 1.54 | 8.70E+03 | 5.60E+03 |
| MID-19898 | 2.20E−02 | 1.53 | 1.30E+03 | 8.40E+02 |
| hsa-miR-29a-3p | 2.00E−02 | 1.52 | 2.60E+04 | 1.70E+04 |
| hsa-miR-30d-5p | 4.30E−03 | 1.51 | 4.10E+03 | 2.70E+03 |
| hsa-miR-664b-3p | 2.60E−02 | 1.49 | 2.20E+03 | 1.50E+03 |
| hsa-miR-30b-5p | 6.50E−03 | 1.48 | 4.90E+03 | 3.30E+03 |
| hsa-miR-1271-5p | 1.10E−03 | 1.47 | 5.00E+02 | 3.40E+02 |
| MID-23291 | 2.10E−02 | 1.45 | 4.20E+03 | 2.90E+03 |
| hsa-miR-505-5p | 1.20E−03 | 1.44 | 5.40E+02 | 3.70E+02 |
| hsa-miR-532-5p | 3.10E−02 | 1.43 | 6.90E+02 | 4.80E+02 |
| hsa-miR-361-5p | 3.40E−05 | 1.41 | 3.60E+03 | 2.60E+03 |
| hsa-miR-504-5p | 4.70E−06 | 1.38 | 3.10E+02 | 2.20E+02 |
| hsa-miR-185-3p | 3.00E−02 | 1.35 | 3.90E+02 | 2.90E+02 |
| hsa-miR-19b-3p | 3.90E−02 | 1.35 | 3.10E+03 | 2.30E+03 |
| MID-00206 | 4.60E−02 | 1.33 | 7.10E+02 | 5.30E+02 |
| hsa-miR-5001-5p | 3.70E−02 | 1.33 | 1.70E+03 | 1.30E+03 |
| hsa-miR-4800-3p | 1.40E−02 | 1.32 | 3.60E+02 | 2.70E+02 |
| MID-18307 | 4.80E−02 | 1.3 | 5.50E+02 | 4.20E+02 |
| hsa-miR-423-5p | 4.40E−03 | 1.3 | 4.60E+03 | 3.50E+03 |
| MID-00713 | 3.50E−02 | 1.28 | 2.00E+03 | 1.50E+03 |
| hsa-miR-4695-5p | 3.30E−02 | 1.27 | 5.90E+02 | 4.70E+02 |
| hsa-miR-30c-2-3p | 1.60E−02 | 1.27 | 3.40E+02 | 2.70E+02 |
| MID-00612 | 3.20E−02 | 1.23 | 3.60E+02 | 2.90E+02 |

^{a}p-values were calculated using a two-sided (unpaired) Student's t-test.
^{b}Fold-change represents the ratio between the median values of each group.
^{c}Median values: median of expression values (rounded).

TABLE 9
MicroRNAs up-regulated in STS that were treated or who had extremely
short survival times (<60 days) versus 1^{st} surgery LTS

| | | | LTS | STS |
|---|---|---|---|---|
| | p- | fold- | median | median |
| microRNA | value^{a} | change^{b} | value^{c} | value^{c} |
| MID-01140 | 1.30E−03 | 23.79 | 4.80E+02 | 1.10E+04 |
| MID-01141 | 5.80E−04 | 19.78 | 3.00E+02 | 5.90E+03 |
| hsa-miR-1290 | 3.50E−03 | 11.82 | 1.30E+02 | 1.50E+03 |
| hsa-miR-155-5p | 4.00E−03 | 6.59 | 3.20E+02 | 2.10E+03 |
| hsa-miR-222-3p | 2.40E−02 | 6.42 | 2.60E+03 | 1.70E+04 |
| hsa-miR-34a-5p | 2.60E−03 | 6.13 | 1.20E+03 | 7.60E+03 |
| hsa-miR-210-3p | 3.20E−02 | 5.97 | 1.10E+03 | 6.60E+03 |
| hsa-miR-221-3p | 2.10E−02 | 5.4 | 3.80E+03 | 2.00E+04 |
| hsa-miR-34c-5p | 5.00E−05 | 4.2 | 1.50E+02 | 6.20E+02 |
| hsa-miR-34b-5p | 8.00E−05 | 3.59 | 1.20E+02 | 4.30E+02 |
| hsa-miR-224-5p | 2.40E−02 | 2.93 | 1.00E+02 | 3.00E+02 |
| hsa-miR-4732-5p | 1.80E−02 | 2.91 | 5.90E+02 | 1.70E+03 |
| hsa-miR-204-5p | 2.90E−02 | 2.64 | 7.20E+02 | 1.90E+03 |
| hsa-miR-129-2-3p | 5.60E−03 | 2.61 | 5.60E+02 | 1.50E+03 |
| hsa-miR-34a-3p | 5.40E−03 | 2.42 | 1.20E+02 | 3.00E+02 |
| hsa-miR-584-5p | 5.10E−03 | 2.37 | 1.40E+02 | 3.20E+02 |
| hsa-miR-212-3p | 1.50E−02 | 2.22 | 1.50E+02 | 3.30E+02 |
| hsa-miR-487b-3p | 2.50E−02 | 2.05 | 6.60E+02 | 1.30E+03 |
| hsa-miR-451a | 4.10E−02 | 2.04 | 2.80E+04 | 5.70E+04 |
| hsa-miR-330-3p | 4.60E−02 | 1.93 | 4.10E+02 | 7.80E+02 |
| hsa-miR-132-3p | 1.60E−02 | 1.9 | 1.00E+03 | 2.00E+03 |
| hsa-miR-339-5p | 2.30E−02 | 1.9 | 2.50E+02 | 4.70E+02 |
| hsa-miR-455-3p | 3.40E−02 | 1.89 | 4.90E+02 | 9.30E+02 |
| hsa-miR-199a-3p | 1.40E−02 | 1.89 | 7.00E+02 | 1.30E+03 |
| hsa-miR-323a-3p | 2.60E−02 | 1.89 | 2.60E+02 | 4.90E+02 |
| MID-19433 | 1.30E−02 | 1.88 | 8.60E+04 | 1.60E+05 |
| MID-14865 | 3.90E−03 | 1.82 | 9.50E+04 | 1.70E+05 |
| hsa-miR-129-1-3p | 3.20E−03 | 1.81 | 4.00E+02 | 7.20E+02 |
| hsa-miR-329-3p | 3.60E−02 | 1.81 | 2.80E+02 | 5.10E+02 |
| hsa-miR-5100 | 7.80E−03 | 1.79 | 1.90E+05 | 3.40E+05 |
| MID-15867 | 8.80E−03 | 1.79 | 7.60E+03 | 1.40E+04 |
| hsa-miR-671-5p | 2.50E−03 | 1.78 | 3.70E+02 | 6.70E+02 |
| hsa-miR-4521 | 2.80E−02 | 1.77 | 2.30E+02 | 4.10E+02 |
| hsa-miR-132-5p | 4.10E−02 | 1.77 | 2.70E+02 | 4.90E+02 |
| hsa-miR-503-5p | 1.10E−02 | 1.69 | 2.00E+02 | 3.30E+02 |
| hsa-miR-3200-3p | 1.10E−02 | 1.66 | 2.30E+02 | 3.80E+02 |
| hsa-miR-18a-5p | 5.00E−03 | 1.65 | 4.80E+02 | 7.90E+02 |
| hsa-miR-142-3p | 3.70E−02 | 1.65 | 3.70E+02 | 6.20E+02 |
| hsa-miR-6723-5p | 1.90E−02 | 1.6 | 5.10E+02 | 8.10E+02 |
| hsa-miR-4454 | 4.90E−03 | 1.59 | 4.10E+05 | 6.50E+05 |
| MID-16752 | 1.50E−02 | 1.58 | 2.80E+05 | 4.50E+05 |
| hsa-miR-214-3p | 1.80E−02 | 1.57 | 3.40E+02 | 5.30E+02 |
| hsa-miR-18b-5p | 4.30E−03 | 1.57 | 1.90E+02 | 3.00E+02 |
| hsa-miR-625-5p | 2.40E−02 | 1.53 | 3.00E+02 | 4.60E+02 |
| hsa-miR-185-5p | 2.10E−03 | 1.53 | 3.30E+03 | 5.00E+03 |
| MID-24702 | 7.30E−03 | 1.52 | 2.40E+05 | 3.60E+05 |
| MID-19434 | 6.20E−03 | 1.48 | 7.40E+04 | 1.10E+05 |
| hsa-miR-425-5p | 1.40E−02 | 1.46 | 1.00E+03 | 1.50E+03 |
| hsa-miR-769-5p | 1.00E−02 | 1.46 | 2.90E+02 | 4.20E+02 |
| hsa-miR-4306 | 4.50E−03 | 1.4 | 7.20E+02 | 1.00E+03 |
| hsa-miR-107 | 4.50E−02 | 1.32 | 2.50E+04 | 3.20E+04 |
| hsa-miR-199a-5p | 4.40E−02 | 1.29 | 8.70E+02 | 1.10E+03 |
| hsa-miR-99b-3p | 3.20E−02 | 1.23 | 4.00E+02 | 4.90E+02 |

^{a}p-values were calculated using a two-sided (unpaired) Student's t-test.
^{b}Fold-change represents the ratio between the median values of each group.
^{c}Median values: median of expression values (rounded).

### Example 8

### New PG-NH₂-Derivatives Pegylated or FITC-Labeled

A number of new PG-NH₂-derivatives were synthesized, which carried polyethylene glycol (PEG) and/or fluorescein isothiocyanate (FITC) in substitution for the amine group.

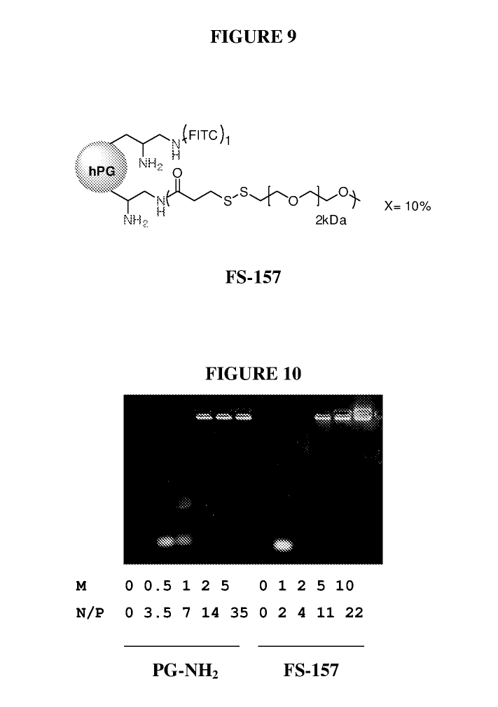

FS-157 is the compound that showed the best performance as a microRNA carrier and in intracellular trafficking, and it is schematically presented in FIG. 9. FS-157 is a FITC-labeled PG-NH₂—SS-PEG. In this compound, 10% of amines were shielded with PEG, and its total molecular weight was of 2 kDa. The PEG moiety was linked via a bioreducible, disulphide (S—S) bond, which was introduced so that the PEG shell would be cleavable under reductive intracellular environment.

An electrophoresis mobility-shift assay (EMSA) of the new PG-NH₂-derivative FS-157 in the presence of hsa-miR-34a is shown in FIG. 10. Fifty (50) pmol of hsa-miR-34a was incubated in the presence of the PG-NH₂-derivative at increasing ratios of nanocarrier:microRNA for 15 minutes at room temperature (RT). Mobility of free and nanocarrier-complexed microRNA was analyzed by agarose gel electrophoresis at N/P ratios of 0, 3.5, 7, 14, 35 for PG-NH₂ and N/P ratios of 0, 2, 4, 11, 22 for FS-157.

### Example 9

### Reporter Activity of the New PG-NH₂-Derivative-hsa-miR-34a

The biological activity of the miR-PG-NH₂-derivative polyplex was evaluated using a reporter assay (psiCHECK™-2, Promega) in HeLa cells.

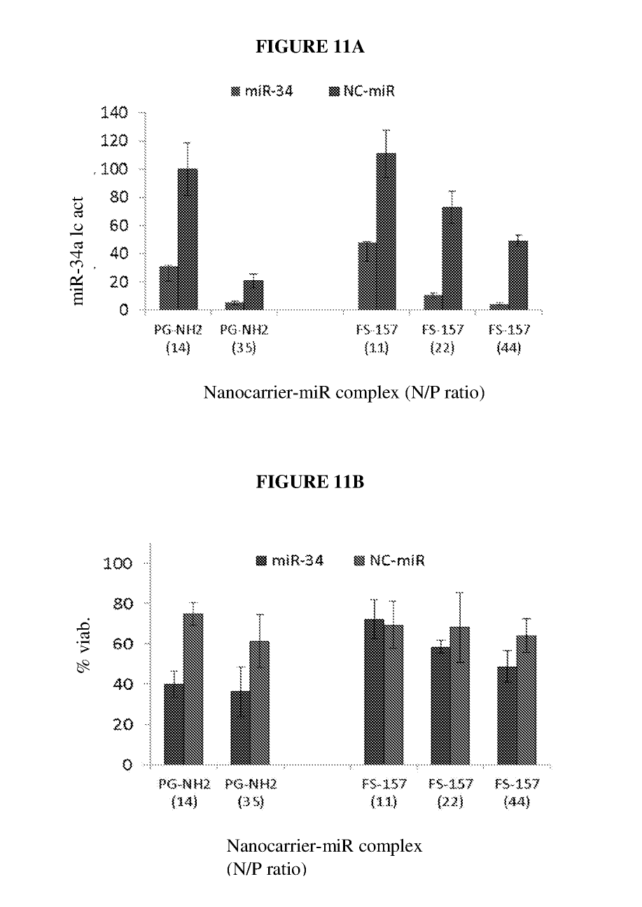

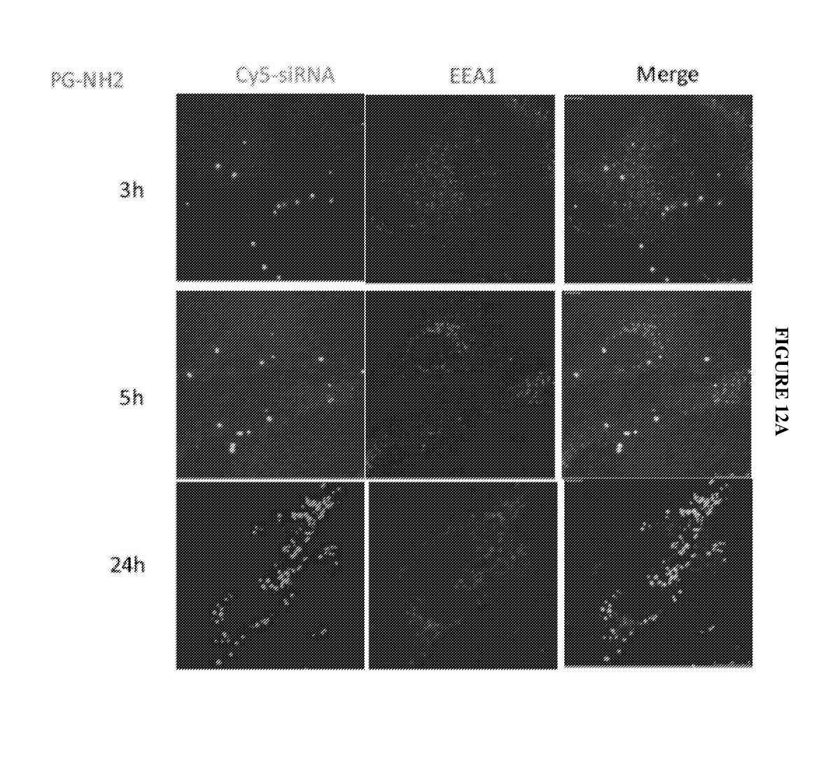

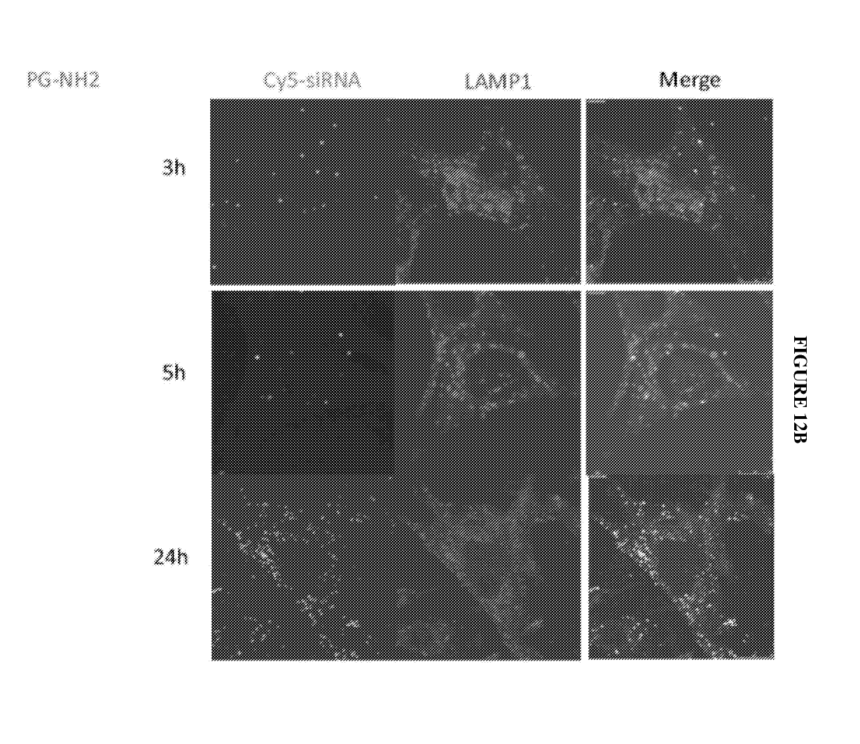

HeLa cells were transfected with hsa-miR34-psiCHECK reporter plasmid (4 μg plasmid into a 10 cm plate). 24 hours later, cells were re-plated in a 96-well plate and treated after 5 hours with PG-NH₂-derivatives-miRNA polyplexes (200 nM miRNA complexed with nanocarrier according to the indicated N/P ratios). Following 72 hours, cells were harvested and assayed for Renilla and firefly luciferase activities. The hsa-miR-34a-regulated Renilla luciferase activity was normalized to firefly luciferase, transcribed under a constitutive promoter. Results are presented in FIGS. 11A-B.

The results show that the hsa-miR-34a which is delivered into the cell with the PG-NH₂-miR-34a or FS-157-miR-34a complexes is capable of inducing expression of the transcription reporter (FIG. 11A), meaning that the hsa-miR-34a delivered into the cell through the nanocarrier is active. FIG. 11B shows cell viability further to PG-NH₂-miR-34a or FS-157 conjugated to hsa-miR-34a transfection.

### Example 10

### Intra-Cellular Trafficking of PG-NH₂-Derivative

PG-NH₂-miR-34a and FS-157 conjugated to hsa-miR-34a were tested for intra-cellular trafficking, regarding their endosomal release/escape and uptake, as well as lysosomal uptake.

U87 MG cells were seeded in coverslips (1×10⁵ cells/well). After 5 hours cells were treated with 100 nM Cy5-labeled siRNA complexed with PG-NH₂ (N/P 7), FS-148b (N/P 24), FS-157 (N/P 22) or FS-158 (N/P 22). Cells were fixed for 3, 5 and 24 hours following treatment.

Endosome staining was achieved using the expression of protein EEA1 as marker. Cells were fixed 20 minutes with paraformaldehyde and permeabilized for 10 minutes with 0.1% Triton-X. Lysosome staining was achieved using the expression of protein LAMP1 as marker. Cell fixation and permeabilization was obtained by treatment in cold methanol for 10 minutes.

Cell slides were immunostained with anti-EEA1 (BD-610456) and anti-LAMP1 antibodies (Cell Signaling D2D11), followed by rhodamine-labeled goat anti-mouse and goat anti-rabbit secondary antibodies, respectively.

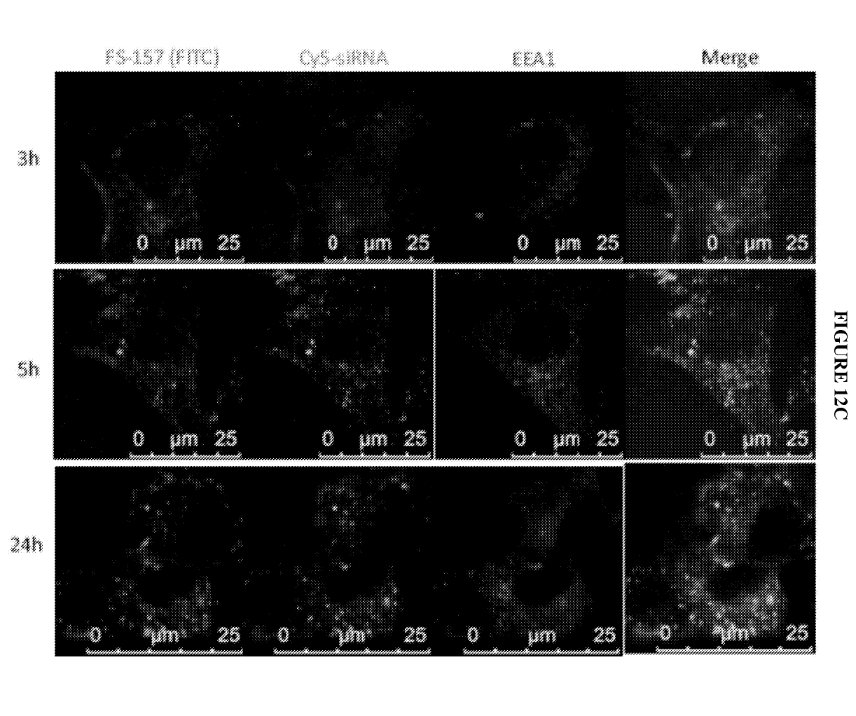

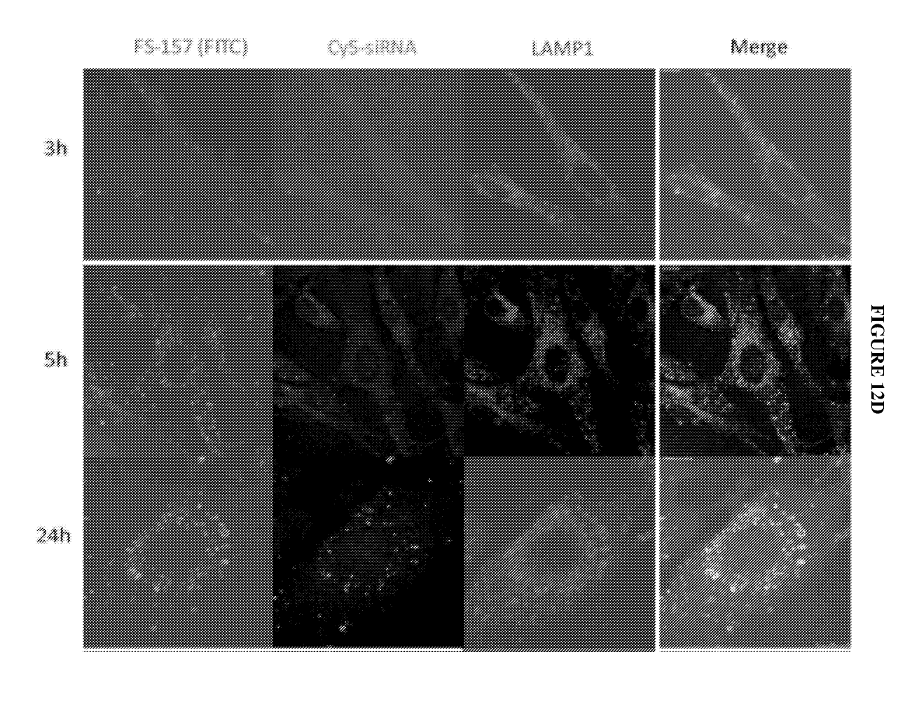

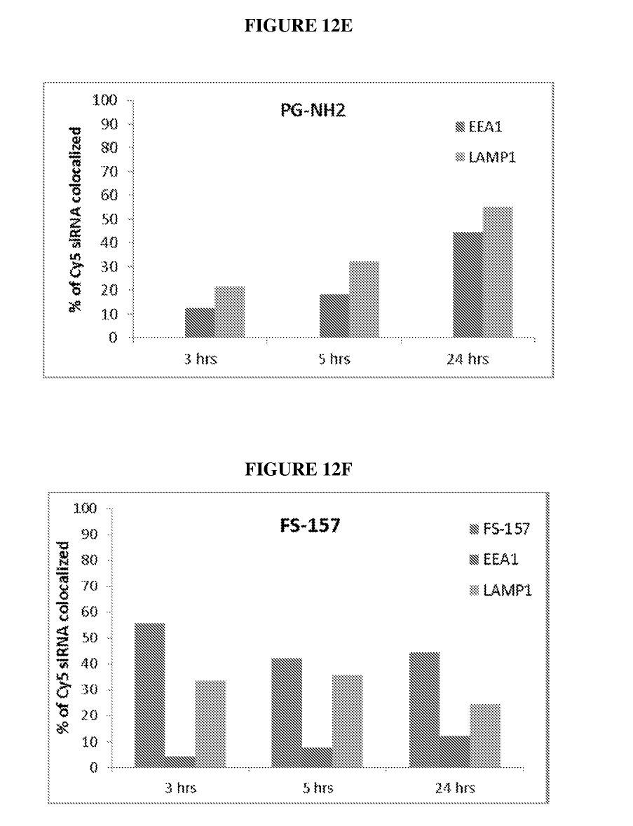

Cellular uptake and internalization were monitored by confocal microscopy, using a Leica TCS STED confocal imaging system (Leica Microsystems, Wetzlar, Germany). Results are presented in FIGS. 12A-12F.

The results show that there is cellular uptake through endocytosis, a fraction is sequestered to the endosomal compartment and a fraction of the complex is released to the cytoplasm. This fraction released is enough to provide activity, as observed in the psi-CHECK reporter assay. Anther fraction of the system, or of the microRNA, is sequestered into the lysosomal compartment.

1. A system comprising at least one nanocarrier and at least one nucleic acid molecule, said nanocarrier being a compound having a structure according to formula (I), 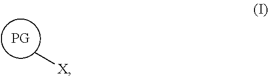
wherein PG denotes a linear or branched polyglycerol core, and X is covalently bound to a carbon atom of the polyglycerol core and is independently selected at each instance from the group consisting of (a) —NR¹R², (b) —OC(═O)—NR³R⁴, (c) —NH—C(O)—CH₂CH₂—S—S—[CH₂CH₂O]_{q}—Y and (d) —CH(NH₂)—CH₂—NH—C(O)—CH₂CH₂—S—S—[CH₂CH₂O]_{q}—Y, wherein at each occurrence q is independently 20-50 and Y is independently H or CH₃,
wherein the polyglycerol core carries a plurality of groups of the type X,
R¹ is (i) H, (ii) linear or branched C₁-C₁₀-alkyl which may be substituted and/or interrupted by one or more oxygen, sulphur and/or nitrogen atoms, or by a group R³, or (iii) a group R³;
R² is (i) H, (ii) linear or branched C₁-C₁₀-alkyl which may be substituted and/or interrupted by one or more oxygen, sulphur and/or nitrogen atoms, or by a group R³, or (iii) R³;
R³ is —(CH₂CH₂NH)ₙ—H, wherein n is 1-100;
R⁴ is H or C₁-C₄-alkyl which may be substituted and/or interrupted by one or more oxygen, sulphur and/or nitrogen atoms;
and said nucleic acid molecule comprises a sequence denoted by;
(a) any one of SEQ ID NO.1-145 and 152-299, or a sequence at least about 80% identical to any one of SEQ ID NO.1-145 and 152-299; or
(b) a sequence complementary to a sequence denoted by any one of SEQ ID NO. 1-145, or to a sequence at least 80% identical to any one of SEQ ID NO.1-145.

2. (canceled)

3. The system of claim 1 wherein about 10% of the X groups have a structure selected from —NH—C(O)—CH₂CH₂—S—S—[CH₂CH₂O]ₘ—Y and —CH(NH₂)—CH₂—NH—C(O)—CH₂CH₂—S—S—[CH₂CH₂O]_{q}—Y, wherein q is on average 20-50 and Y is H or CH₃.

4. The system of claim 3, wherein q is on average 40-50.

5. The system of claim 4, wherein q is on average 44-45.

6. The system of claim 1, wherein the nanocarrier compound further comprises a fluorescein label.

7. The system of claim 6, wherein said fluorescein label is attached to the PG core via a bond formed between an amine moiety pendant from the PG core and an isothiocyanate unit covalently attached to the fluorescein.

8. The system of claim 7, wherein not more than ten, not more than nine, not more than eight, not more than seven, not more than six, not more than five, not more than four, not more than three, not more than two or fluorescein moieties are attached to the PG core.

9. The system of claim 7, wherein a single fluorescein moiety is attached to the PG core.

10. A system comprising at least one nanocarrier and at least one nucleic acid molecule, said nanocarrier being a compound having a structure according to formula (I), 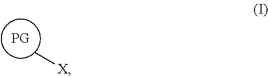
wherein PG denotes a linear or branched polyglycerol core, and X is covalently bound to a carbon atom of the polyglycerol core and is at each instance —NR¹R²,
wherein the polyglycerol core carries a plurality of groups of the type X,
R¹ is independently at each instance (i) H, (ii) linear or branched C₁-C₁₀-alkyl which may be substituted and/or interrupted by one or more oxygen, sulphur and/or nitrogen atoms, or by a group R³, or (iii) a group R³;
R² is independently at each instance (i) H, (ii) linear or branched C₁-C₁₀-alkyl which may be substituted and/or interrupted by one or more oxygen, sulphur and/or nitrogen atoms, or by a group R³, or (iii) R³;
R³ is —(CH₂CH₂NH)ₙ—H, wherein n is 1-100;
and said nucleic acid comprises a sequence denoted by any one of SEQ ID NO.123, SEQ ID NO.14, SEQ ID NO.117, SEQ ID NO.65, SEQ ID NO.70, SEQ ID NO.122, SEQ ID NO.32, SEQ ID NO.64, SEQ ID NO.63, SEQ ID NO.24, SEQ ID NO.108, SEQ ID NO.130, SEQ ID NO.131, SEQ ID NO.62, SEQ ID NO.15, SEQ ID NO.84 and SEQ ID NO.71, a complement thereof, or a sequence at least about 80% identical to any one of SEQ ID NO.123, SEQ ID NO.14, SEQ ID NO.117, SEQ ID NO.65, SEQ ID NO.70, SEQ ID NO.122, SEQ ID NO.32, SEQ ID NO.64, SEQ ID NO.63, SEQ ID NO.24, SEQ ID NO.108, SEQ ID NO.130, SEQ ID NO.131, SEQ ID NO.62, SEQ ID NO.15, SEQ ID NO.84 and SEQ ID NO.71, or a complement thereof.

11. The system according to claim 1, wherein said nucleic acid is carried by, or bound to, said nanocarrier in any one of a covalent, ionic or complexed manner.

12. A pharmaceutical composition comprising as active agent the system according to claim 1.

13. The pharmaceutical composition according to claim 12, further comprising any one of adjuvants, carriers, diluents and excipients.

14. A method of treating cancer, said method comprising administering a therapeutically effective amount of the system according to claim 1, or a composition comprising the system, to a subject in need thereof.

15. (canceled)

16. The method according to claim 14, wherein said cancer is a brain tumor.

17. A method of inhibiting miR function in a cell, said method comprising contacting said cell with the system according to claim 1 or with a pharmaceutical composition comprising the system, wherein said nucleic acid in said system comprises any one of the sequences denoted by any one of SEQ ID NO.123, SEQ ID NO.14, SEQ ID NO.117, SEQ ID NO.65, SEQ ID NO.70, SEQ ID NO.122, SEQ ID NO.32, SEQ ID NO.64, SEQ ID NO.63, SEQ ID NO.24, SEQ ID NO.108, SEQ ID NO.130, SEQ ID NO.131, SEQ ID NO.62, SEQ ID NO.15, SEQ ID NO.84 and SEQ ID NO.71, or a complement thereof, or a sequence at least about 80% identical to SEQ ID NO.123, SEQ ID NO.14, SEQ ID NO.117, SEQ ID NO.65, SEQ ID NO.70, SEQ ID NO.122, SEQ ID NO.32, SEQ ID NO.64, SEQ ID NO.63, SEQ ID NO.24, SEQ ID NO.108, SEQ ID NO.130, SEQ ID NO.131, SEQ ID NO.62, SEQ ID NO.15, SEQ ID NO.84 and SEQ ID NO.71, or a complement thereof.

18. A method of mimicking miR function in a cell, said method comprising contacting said cell with the system according to claim 1 or with a pharmaceutical composition comprising the system, wherein said nucleic acid in said system comprises any one of the sequences denoted by SEQ ID NO.123, SEQ ID NO.14, SEQ ID NO.117, SEQ ID NO.65, SEQ ID NO.70, SEQ ID NO.122, SEQ ID NO.32, SEQ ID NO.64, SEQ ID NO.63, SEQ ID NO.24, SEQ ID NO.108, SEQ ID NO.130, SEQ ID NO.131, SEQ ID NO.62, SEQ ID NO.15, SEQ ID NO.84 and SEQ ID NO.71, or a complement thereof, or a sequence at least about 80% identical to any one of SEQ ID NO.123, SEQ ID NO.14, SEQ ID NO.117, SEQ ID NO.65, SEQ ID NO.70, SEQ ID NO.122, SEQ ID NO.32, SEQ ID NO.64, SEQ ID NO.63, SEQ ID NO.24, SEQ ID NO.108, SEQ ID NO.130, SEQ ID NO.131, SEQ ID NO.62, SEQ ID NO.15, SEQ ID NO.84 and SEQ ID NO.71, or a complement thereof.

19. The system according to claim 1, for use in the treatment of cancer.

20. The system according to claim 19, wherein said cancer is a brain tumor.

21.-23. (canceled)

24. A kit for the treatment of cancer, said kit comprising:
a. the system according to claim 1;
b. means for administering said system to a patient in need thereof; and
c. optionally instructions of dosage and/or administration of said system.

25. (canceled)
